SEQUENCE LISTING (2 sequences)

SEQ ID NO: 1 - DNA, 21 bp, Artificial Sequence
  Chemically Synthesized
gcgagtaacaagggcatggaa

SEQ ID NO: 2 - DNA, 21 bp, Artificial Sequence
  Chemically Synthesized
cttcaaggtttcgagagttaa

(19) United States
(12) Patent Application Publication
(10) Pub. No.: US 2017/0266166 A1
(43) Pub. Date: Sep. 21, 2017

(54) Compositions and Methods to Inhibit Estrogen Receptor Beta for the Treatment of Renal Cell Carcinoma

(71) Applicant: University of Rochester, Rochester, NY (US)

(72) Inventor: Shu-Yuan Yeh, Rochester, NY (US)

(21) Appl. No.: 15/310,003

(22) PCT Filed: May 15, 2015
(86) PCT No.: PCT/US15/31132

§ 371 Date: Nov. 9, 2016

Related U.S. Application Data
(60) Provisional application No. 61/994,836, filed on May 17, 2014.

(51) Int. Cl. A61K 31/436; C12Q 1/68; G01N 33/574; A61K 45/06

(52) U.S. Cl. CPC A61K 31/436; A61K 45/06; C12Q 1/6886; G01N 33/57438; G01N 2800/56; C12Q 2600/158; C12Q 2600/112

(57) ABSTRACT

The invention provides compositions and methods for treating and preventing cancer. The invention relates to compositions and methods for treating subjects having a cancer associated with overexpression of estrogen receptors, especially in males. The invention comprises inhibitors of ERβ, inhibitors of TGFβ-1, inhibitors of SMAD, or inhibitors of HIF, or a combination thereof. In one embodiment, the invention provides a method of treating renal cancer, including renal cell carcinoma.

### CROSS-REFERENCE TO RELATED APPLICATIONS

This application claims priority to U.S. Provisional Patent Application No. 61/994,836 filed May 17, 2014, the contents of which are incorporated by reference herein in their entirety.

### STATEMENT REGARDING FEDERALLY SPONSORED RESEARCH OR DEVELOPMENT

This invention was made with government support under grant number CA137474 awarded by the National Institutes of Health (NIH), and URMC Urology Research seed fund. The government has certain rights in the invention.

### BACKGROUND OF THE INVENTION

Renal cell carcinoma (RCC) accounts for approximately 3% of adult malignancies and is the third leading cause of death among urological tumors (Comperat et al., Diagn Intery Imaging, 2012, 93:221-31). Worldwide incidence and mortality rates of RCC are steadily rising at a rate of approximately 2-3% per decade (Gupta et al., Cancer Treat Rev, 2008, 34:193-205). It is estimated that approximately 25-30% of RCC patients have metastases at the time of diagnosis. Even after resection of the primary tumor by radical or partial nephrectomy, relapse occurs in 20-30% of RCC patients (Whelan et al., EAU Update Series, 2003, 1:237-46). While etiological factors of RCC remains largely unknown to date (Murai et al., Curr Opin Urol, 2004, 14:229-33), known risk factors include obesity, diabetes, hypertension, cigarette smoking, diet, as well as oophorectomy and parity in women (Asal et al., Cancer Detect Pref, 1988, 11:359).

RCC is considered to be an immunogenic tumor and a number of immunotherapeutic approaches have been exploited (Ko et al., Clin Cancer Res, 2009, 15:2148-2157). There is only partial or limited benefit obtained when selective RCC patients receive immunotherapies (high dose IL-2, IFNα+bevacizumab) or targeted therapies (tyrosine kinase inhibitors, mTOR inhibitors) (Minarak et al., Immunol Lett, 2013, 152:144-150). The growth of RCC tumor cells is greatly influenced by the immune system (Finn, New Engl J Med, 2008, 358:2704-2715). RCC tissues exhibit a prominent infiltration of immune cells, consisting of T cells, natural killer (NK) cells, dendritic cells (DCs) and neutrophils. Despite profoundly infiltrated immune cells, RCC is not generally eliminated by different immune effector cells. Furthermore, immune dysfunction is found in some RCC patients, which likely contributes to tumor invasion (Minárak et al., Immunol Lett, 2013, 152:144-150). It is of clinical interest to investigate how the tumor cells become resistant and escape from immune system control, and how the tumor associated immune cells influence the RCC development.

Evidences from epidemiological and pathological studies suggest that the neutrophil to lymphocyte ratio is an important prognostic factor based on a RCC study within a large European cohort (Picher et al., Brit J Cancer, 2013, 108:901-907). Neutrophils are one of the key tumor-infiltrating myeloid cells to influence tumor progression (Tazzyman et al., Int J Exp Pathol, 2009, 90:222-231). Similarly to the myeloid macrophages, neutrophils also contain a subpopulation of neutrophils named tumor-associated neutrophils (TAN) (Fridlender et al., PLoS One, 2012, 7:e31524). However, the potential relationship between TAN infiltration and human cancer prognosis has not been systematically discussed (Gregory et al., Cancer Res, 2011, 71:2411-2416). Epidemiological evidence has suggested that neutrophil infiltration within human cancers may be associated with a poor clinical outcome, as observed in patients with metastatic clear cell carcinomas and hepatocellular carcinoma (Donskov, Semin Cancer Biol, 2013, 23(3):200-207).

Early studies indicated that the gender difference with male:female ratio in RCC incidence is 1.6:1.0 (Feldman et al., Oncology, 2006, 20:1745-53), suggesting that sex hormones and/or their receptors may play important roles for the development of RCC. Although this is still debatable, RCC has been revealed to be dependent on estrogen (Concolino et al., Cancer Res, 1978, 38:4340-4). Treatment with synthetic estrogen can induce RCC in animal models (Wolf et al., Carcinogenesis, 1998, 19:2043-7).

Estrogens play important physiological roles in humans. In the adult females estrogen prepares the uterus to accept the fertilized egg and help maintain pregnancy. It also induces proliferation of mammary tissues, prior to the induction of lactation by progesterone and prolactin. Estrogen exposure lowers the risk of heart attack and osteoporosis, but conversely, estrogen exposure stimulates the growth of breast and uterine tissues. Several epidemiological studies have shown that chronic exposure to estrogen is associated with the development of breast cancer. Similarly, women who have either been treated with diethylstilbestrol (Henderson et al., Cancer Res, 1988, 48:246-53) or have been exposed to the estrogenic pesticide dichloro-diphenyl-trichloro-ethane (Wolff et al., J Natl Cancer I, 1993, 85:648-52) are at an increased risk of developing breast cancer. The bioeffects of estrogens are evident through their binding to estrogen receptors (ERs) and subsequent regulation of the transcription and activation of downstream genes.

There are two subtypes of ERs, ER alpha (ERα) and ER beta (ERβ). Distribution of ERα and ERβ varies in different tissue types (Fox et al., Steroids, 2008, 73:1039-51). The correlation between ERα and breast cancer has been extensively studied. ERβ is expressed in the epithelial compartment of the gland and may regulate epithelial proliferation and differentiation (Imamov et al., P Natl Acad Sci USA, 2004, 101:9375-80). However, the detailed mechanisms of ERβ in breast cancer and in other types of cancers remain under-studied. Although the structures of ERα and ERβ are similar, their histological distributions and biological functions are not the same.

Our new data have shown that ERβ signals could affect RCC development. While some therapeutic avenues are currently available for treatment of RCC, their overall success rate is rather limited and unsatisfactory. Thus, there is a need to develop new therapies for RCC, including but not limited to compositions and methods for RCC treatment. The present invention satisfies this unmet need.

### SUMMARY OF THE INVENTION

In one aspect the present invention provides a method for treating cancer associated with abnormal estrogen receptor β (ERβ) expression in a subject. The method comprises administering to the subject a therapeutically effective amount of a composition comprising an inhibitor of an estrogen receptor. In one embodiment, the subject is male. In one embodiment, the cancer is renal cell carcinoma (RCC). In one embodiment, the cancer is sarcomatoid type renal cancer. In one embodiment, the renal cell carcinoma is stage II RCC, stage III RCC, or stage IV RCC.

In one embodiment, the cancer involves one or more cell types selected from the group of clear cells, papillary cells (Types 1 and 2), chromophobe cells, oncocytic cells, collecting duct cells, and transitional cells.

In one embodiment, the inhibitor is a nucleic acid, a siRNA, an antisense nucleic acid, a ribozyme, a peptide, a small molecule, an antagonist, an aptamer, or a peptidomimetic. In one embodiment, the inhibitor is an inverse agonist to ERβ. In one embodiment, the inhibitor is an antagonist to ERβ.

In one embodiment, the inhibitor is a selective estrogen receptor modulator (SERM), steroidal aromatase inhibitor, or non-steroidal aromatase inhibitor.

In one embodiment, the inhibitor further comprises a pharmaceutically acceptable formulation that includes a carrier and optionally one or more excipients, stabilizers, or additives.

In one embodiment, the inhibitor is administered in combination with another therapeutic agent. In one embodiment, the inhibitor is administered in combination with a TGFβ-1 inhibitor. In one embodiment, the inhibitor is administered in combination with a SMAD inhibitor. In one embodiment, the inhibitor is administered in combination with a HIF inhibitor. In one embodiment, the inhibitor is administered in combination with rapamycin.

In one embodiment, the inhibitor is administered orally, topically, transdermally, parenterally, subcutaneously, intravenously, intramuscularly, intraperitoneally, by intranasal instillation, by intracavitary or intravesical instillation, intraocularly, intraarterially, intralesionally, or by application to mucous membranes.

In one embodiment, the inhibitor is a small molecule selected from the group consisting of 7α-[9-(4,4,5,5,5-pentafluoropentylsulfinyl)nonyl]estra-1,3,5(10)-triene-3,17β-diol (also known as ICI 182,780), N-n-butyl-N-methyl-11-(3,17β-dihydroxyestra-1,3,5(10)-triene-7α-yl)undecanamide (also known as ICI 164,384), 11β-[4-[2-(dimethylaminoethoxyl]phenyl]-estradiol (also known as RU 39411), N-methyl-N-isopropyl-(3,17β-dihydroxy-estra-1,2,5(10)-trien-11-β-yl)-undecamide (RU 51625), its 17α-ethynyl derivative (RU 53637), [6-hydroxy-2(4-hydroxyphenyl)benzo[b]thien-3-yl][4-[3-(1-pyrrolidin-yl)ethoxy]phenyl]methanone (LY117018), tamoxifen, toremifene, fulvestrant, raloxifene, raloxifene hydrochloride, clomiphene, keoxifene, 3-[4-(1,2-Diphenylbut-1-enyl)phenyl]acrylic acid and SR-16388.

In one aspect, the present invention provides a method of determining the stage of renal cancer in a subject. The method comprises obtaining a sample comprising renal cancer cells from the subject and determining the level of expression of the ERβ in the renal cancer cells wherein elevated levels of ERβ is an indication of renal cancer progression.

In one aspect, the present invention provides a method of diagnosing renal cancer in a subject. The method comprises obtaining a sample comprising renal cancer cells from the subject and determining the level of expression of the ERβ in the renal cancer cells wherein elevated levels of ERβ is an indication of renal cancer.

In one aspect, the present invention provides a kit for preventing or delaying the onset of cancer in a subject. The kit contains a composition comprising at least one inhibitor of an estrogen receptor. In one embodiment, the kit further comprises one or more of a TGFβ-1 inhibitor, a SMAD inhibitor, and a HIF inhibitor.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1, comprising FIGS. 1A-1C, depicts the results of experiments demonstrating the expression of estrogen receptor β (ERβ) in the different stage/grade of human renal cell carcinoma (RCC) tissues. FIG. 1A shows immunohistochemical (IHC) staining of ERβ expression in low and high stages/grade of human RCC specimens. The ERβ showed a nuclear staining signal. Higher ERβ signals were detected in T3/G3 RCC patient samples. FIG. 1B shows IHC staining data of ERβ protein levels in different stage or grade of RCC tissues. T2-3 RCC tissues (57%) showed a significantly higher ERβ positive rate compared to T1 tissues (18%). Similarly, G2-3 RCC tissues (49%) showed a significantly higher ERβ positive rate compared to G1 tissues (21%). *P<0.05. FIG. 1C shows analysis of TCGA database: RCC patients with positive ERβ expression have a worse clinical outcome with reduced 5 years survival rate. There are a total of 88 RCC cases: 28 RCC cases have ERβ expression with 9 of 28 (32.1%) dead in five years. The rest of the 60 cases have no (or little) ERβ expression, and 6 of the 60 (10.0%) were dead in five years of follow-up.

FIG. 2, comprising FIGS. 2A-2F, depicts the results of experiments comparing ERβ expression in RCC cell lines and estrogen/ERβ effects on RCC cell growth. FIG. 2A shows western blot (upper panel) and qPCR (lower panel) detection of ERα and ERβ in different RCC cell lines. Expression of ERα was not detected in tested RCC cells. ERβ has the highest expression level in 786-O and lowest in A498. Breast cancer cell MCF-7 was used as a positive control. FIG. 2B shows estrogen/ERβ effects on RCC cell growth. The growth rate of 786-O cells (with high endogenous ERβ) is increased in the presence of 17β-estradiol (10 nM) as compared with the control Etoh). FIG. 2C shows growth rate of A498 cells (with low endogenous ERβ) was not significantly changed by estrogen treatment. FIGS. 2D-2E show changes in ERβ expression levels can affect the RCC cell growth. A lentivirus system was used to knock-down ERβ in RCC 786-O cells (by lenti-ERβ-shRNA), or to increase ERβ in A498 cells (by lenti-ERβ cDNA). Cell growth rates were determined by MTT assay. FIG. 2F colony formation assays showed ERβ knock-down in 786-O could inhibit cell growth; overexpression of ERβ in A498 (with low endogenous ERβ) cells can increase cell growth.

FIG. 3, comprising FIGS. 3A-3F, depicts the results of experiments demonstrating that ERβ promotes the migration and invasion of RCC cells. FIG. 3A illustrates the experimental setting for RCC migration. FIG. 3B shows higher ERβ expression promotes RCC cell migration. ERβ knock-down in 786-O cells (786-O sh-ERβ) inhibits cell migration as compared to 786-O sh-Luc cells. ERβ over-expression in A-498 cells (A-498 ERβ) can increase the cell migration capability. Quantification at right. FIG. 3C shows the migration rate of ERβ positive RCC cells (786-O sh-Luc) is further increased in the presence of E2. In comparison, E2 has little effects on migration of 786-O shERβ cells. FIG. 3D shows treatment can effectively promote the migration of A-498 ERβ cells (compared to A-498.vec cells). FIG. 3E illustrates the experimental setting for RCC invasion. FIG. 3F shows higher ERβ expression promotes RCC cell invasion. ERβ knockdown in 786-O cells (786-O sh-ERβ) inhibits cell invasion as compared to 786-O sh-Luc cells. ERβ over-expression in A-498 cells (A-498.ERβ) can increase the cell invasion capability. Quantifications of cell numbers that passed through the transwell are shown on the right panel.

FIG. 4, comprising FIGS. 4A-4D, depicts the results of experiments demonstrating that ERβ induces the epithelial-mesenchymal transition (EMT) morphology and promotes EMT marker expression via microRNA in RCC. FIG. 4A shows changes in ERβ expression lead to correlative EMT morphology changes in RCC cells. Cells became slender when ERβ is knocked-down in 786-O cells (sh-ERβ vs sh-Luc), or became spindle shaped with addition of ERβ in A498 cells (A-498.ERβ vs A-498 vec). The correlative morphology changes suggest a mesenchymal-like shape when RCC cells have a higher ERβ levels. FIG. 4B shows higher ERβ increases EMT marker expressions in RCC. In 786-O cells, 786-O sh-ERβ cells versus 786-O sh-Luc cells were compared. The higher ERβ expression and morphology changes are accompanied with the changed expressions of EMT markers with increased levels of N-Cadherin, snail, twist and vimentin as well as reduced E-Cadherin. FIG. 4C shows immunofluorescence staining of N-Cadherin in 786-O and A498 cells with high vs. low ERβ. Nuclei were visualized with DAPI staining. FIG. 4D shows real-time PCR of quantification of metastasis related miRNA in RCC cells. Comparisons were made in 786-O cells (sh-ERβn vs. sh-Luc) and A498 cells (ERβ vs. vec).

FIG. 5, comprising FIGS. 5A-5F, depicts the results of experiments demonstrating that the TGFβ-1/SMAD3 signal was a key player for ERβ-mediated proliferation and migration in RCC cells. FIG. 5A shows a Q-PCR array to screen metastasis related genes in 786-O sh-ERβ/sh-Luc and A498 ERβ/Vec cells. Q-PCR results showed that higher ERβ expression could have higher mRNA levels of TGFβ-1 and SMAD3 in A498 ERβ (vs A498 vec) and 786-O shLuc (vs 786-O sh-ERβ) cells. FIG. 5B shows expression of ERβ, TGFβ-1, and SMAD3 protein levels in 786-O sh-ERβ/sh-Luc, and A498 ERβ/Vec cells were detected by Western blot. FIG. 5C shows IHC staining of RCC clinical samples and the correlative higher expression of ERβ and TGFβ-1 by comparing with G3 vs G1. FIG. 5D shows TGFβR-1 inhibitor can reverse higher ERβ-mediated signal changes. Western blot assay was applied to confirm the expression of TGFβ-1 and reduced pSMAD3 after treatment of TGFβR-1 inhibitor. FIG. 5E illustrates the experimental setting for treatment of RCC with TGFβR-1 inhibitor and migration. RCC cells were cultured for 48 hr and then treated with 10 μM TGFβR-1 inhibitor for 6 hr. RCC cells were then trypsinized and seeded in the upper transwell (5×10⁴/well) for 24 hr to determine the migration rate. FIG. 5F shows TGFβR-1 inhibitor treatment can attenuate higher ERβ-mediated RCC migration. The migration abilities of higher ERβ expressed RCC cells (786-O sh-Luc and A498 ERβ) were dramatically attenuated upon treatment with 10 μM TGFβR-1 inhibitor for 48 hr. Quantification of migrated cells was shown on the right panel.

FIG. 6, comprising FIGS. 6A-6D, depicts the results of experiments demonstrating that ERβ promotes RCC growth and metastasis in subcutaneous and orthotopic xenografted mouse models. FIG. 6A shows subcutaneous or orthotopic implantation of RCC cells in male nude mice models. Two pairs of RCC cells: 786-O sh-Luc/sh-ERβ and A498 Vec/ERβ were orthotopically implanted into renal capsule or injected subcutaneously, respectively (N=6 mice per group). FIG. 6B shows subcutaneous or orthotopic implantation of RCC cells in female nude mouse models. In 8 week old female nude, RCC cells with high vs low ERβ were implanted. The gross appearances, tumor volume and weight quantification were analyzed. The A498.ERβ cells grow a bigger tumor than the A498 vector controls. Similar results were also found in mice receiving 786-O sh-ERβ/sh-Luc cells both in subcutaneous or orthotopic implantations. RCC tumor grafts with higher ERβ develop distal metastatic tumors. FIG. 6C shows higher ERβ expression could stimulate RCC distal metastasis. Two out of 6 mice with A498 ERβ cells formed diaphragm, liver and hepatic hip lymph node metastasis and none of A498 Vec group had metastasis. One out of 6 mice in 786-O sh-Luc formed distal metastasis, and none of the 786-O sh-ERβ group had metastasis. Representative metastasis images were shown, and red arrows indicated diaphragm and liver metastasis. FIG. 6D shows anti-estrogens, PHTPP and ICI 182,780, inhibit the RCC tumor growth and metastasis. IVIS imaging results show PHTPP and ICI 182,780 treatments inhibit the growth and metastasis of RCC in vivo. Mice tumor weights were measured.

FIG. 7, comprising FIGS. 7A-7B, depicts a summary of invasion and metastasis related gene transcription profile in 786-O shERβ/shLuc and A498 hERβ/Vec cells using a focused Q-PCR array. FIG. 7A shows a summary of the samples investigated. The investigate the expression of estrogen receptors in human RCC tissue, malignant tissue specimens were collected from 80 patients who had undergone partial nephrectomy or radical nephrectomy for primary renal cell carcinoma at the Department of Urology, the First Affiliated Hospital of Medicine School, Xi'an Jiaotong University, between 2002 and 2012. All tumor tissues were diagnosed according to the 2009 edition of the TNM system and nuclear grading was performed according to WHO guidelines. Tissues were fixed in 4% paraformaldehyde and embedded in paraffin. Five-micrometer-thick sections were prepared and immunohistochemical staining against estrogen receptors was performed using a Dako Autostainer Plus system. FIG. 7B shows the transcriptional levels of TGFβ-1 and SMAD3 were dramatically increased in A498 hERβ cells as compared to A498 Vec cells, and the transcriptional levels of those two genes were found significantly down-regulated in 786-O shERβ cells as compared to 786-O shLuc cells.

FIG. 8 depicts a diagram illustrating that ERβ regulates the TGFβ/SMAD pathway in RCC. ERα expression is constantly negative in RCC tumors.

FIG. 9 depicts the results of experiments demonstrating that the anti-estrogen, tamoxifen, could not be as effective as ICI 182,780 and PHTPP to inhibit the RCC tumor growth and metastasis. Compared to the control group, tamoxifen could only inhibit about 40% of tumor size, which is not as effective as the PHTPP and ICI 182,780 treatment on the growth and metastasis of RCC in vivo. Results of IVIS imaging, tumor size, and tumor weights are shown and compared.

FIG. 10, comprising FIGS. 10A-10D, depicts the results of experiments demonstrating that compared to non-malignant kidney cells, RCC cells recruit more neutrophils. FIG. 10A. Human clear cell RCC specimens were stained for CD66G to detect neutrophils (upper panels). IHC staining results showed that more CD66G+ signals, which suggested more neutrophil cells infiltrations, in the renal cancer area than in the adjacent normal kidney areas. To investigate the expression of CD66G in human RCC tissues, malignant tissue specimens along with 15 adjacent normal tissues were collected from 60 patients who had undergone partial or radical nephrectomy for primary RCC (lower panels). All tumor tissues were diagnosed according to the 2009 edition of the TNM system and nuclear grading was performed according to WHO guidelines. The clinic-pathological parameters of these tumors are listed in FIG. 10D. FIG. 10B. After treating with 1.25% DMSO for 5 days, HL-60 cells were differentiated to N2 type neutrophils, HL-60N. qPCR was applied to validate neutrophil marker (CD66G) and N2 type markers (CD11b, NPO, and hARg-1). FIG. 10C. RCC cells can better attract the HL-60N migration/infiltration. 1×10⁵ of RCC cells or non-malignant kidney epithelial cells were plated into the lower chamber of the transwell. 1×10⁵ of HL-60N cells were plated onto the upper chamber with 5 um pore polycarbonate membrane to determine HL-60N migration rate toward CM collected from RCC or non-malignant kidney cells. After 8 hrs, the HL60-N cells migrated into the lower chamber were collected and counted by the Bio-Rad TC10 automatic cell counter. Compared to the normal kidney epithelial cell lines (HKC-2 and HKC-8), RCC cells (786-O and A498) recruited more HL-60N (*P<0.05).

FIG. 11 comprising FIGS. 11A-11B, depicts the results of experiments demonstrating that co-culture with neutrophils promoted RCC invasion. FIG. 11A. The illustration shows the procedure to co-culture RCC and HL-60N cells and to test HL-60N cells promoted RCC migration. The RCC and HL-60N cells were co-cultured in 0.4 μm pore size transwell plates for 7 days. After co-culturing with HL-60N, RCC cells were re-seeded into the upper chamber of a new transwell with 10% FBS fresh media in the bottom chamber. The transwell migration results showed that HL-60N co-cultured RCC cells have a higher migration capability (*P<0.05). FIG. 11B. HL-60N co-cultured RCC cells have a higher invasion capability. Using a similar protocol, it was found that there was a higher invasion ability in RCC cells after co-culture with HL-60N cells than non-co-cultured RCC cells.

FIG. 12, comprising FIGS. 12A-12C, depicts the dissection of the mechanism by which infiltrated neutrophils modulate ERβ expression in RCC cells. FIG. 12A. Screening gene profile changes in neutrophil-co-incubated RCC cells. Q-PCR results showed mRNA expressions of VEGFa and HIF2α, but not VEGFc, VEGFd and HIF1α, were selectively and significantly increased in RCC cells after co-culture with HL-60N cells for 7 days. The data were validated in 2 different RCC cells, 786-O and A498. FIG. 12B. Detection of ERβ levels in different RCC cells. Western blot detection of ERβ in different RCC cell lines. Breast cancer cells, MCF-7, were used as a positive control for ERβ expression. ERβ has the highest expression level in 786-O and the lowest in A498. qPCR was applied to check ERβ expression in RCC cell lines. FIG. 12C. Western blot detection of ERβ, VEGFa and HIF2a protein expressions after co-culture with HL-60N cells. Data showed that co-culture of HL-60N and RCC cells could increase ERβ and other invasion/angiogenesis related genes in RCC cells. GAPDH was used as control to show the equal loading of protein.

FIG. 13, comprising FIGS. 13A-13B, depicts the results of experiments demonstrating that down-regulated ERβ could regulate the down-stream VEGFa and HIF2α pathways in RCC cells. FIG. 13A. We used the lentiviral-shRNA system to knock down ERβ in 786-O (with high endogenous ERβ) and A498 (with low endogenous ERβ), then co-cultured with HL-60N for 7 days. Data showed that knockdown of ERβ expression can reverse HL-60N promoted RCC invasion. FIG. 13B. Western blot data showed that ERβ and the genes of the VEGFa and HIF2α pathway are increased in RCC cells after co-culture with HL-60N. Knockdown of ERβ in RCC could diminish the HL-60N co-culture promoted VEGFa and HIF2α expressions in RCC cells.

FIG. 14, comprising FIGS. 14A-14D, depicts the results of experiments demonstrating that HIF inhibitor and rapamycin treatment can attenuate neutrophils-promoted RCC migration and invasion. FIG. 14A. Using lentiviral system to knockdown ERβ in A498 and 786-O, those RCC cells were then co-cultured with HL-60N. After 5 days of co-culture, 10 μM HIF inhibitor was added for 2 more days. RCC cells were then trypsinized and seeded in the upper chambers of transwells (5×10⁴/well) with 10% FBS media for 24 hr to determine the migration rate. The cartoon illustrates the procedure to perform the experiments. FIG. 14B. qPCR to detect ERβ, VEGFa and HIF2α expression with or without treatment of HIF inhibitor. Together, our data support that a lower ERβ expression can down-regulate VEGFa/HIF2α expression. HIF inhibitor treatment can diminish neutrophils/ERβ-stimulated RCC migration. FIG. 14C, Rapamycin treatment suppresses neutrophil infiltration. CMs collected from 786-O and HK2 cells with/without rapamycin (10 ng/ml) treatment were put in bottom wells of 24 wells cell culture plate. Neutrophils were seeded into upper-transwell (pore size: 5 μm). Infiltrated neutrophil were collected in bottom wells after 3 hrs of incubation. FIG. 14D. Rapamycin treatment could attenuate neutrophil-promoted RCC invasion. In the upper left panel: 786-O alone with DMSO mock treatment; the upper right panel: HL-60 co-cultured with 786-O cells with DMSO mock treatment. In the bottom left panel, 786-O cells with rapamycin treatment; in bottom right panel, HL-60 and 786-O co-culture with rapamycin treatment (10 ng/ml). RCC invasion quantification results were averaged from counting six represented field under microscope of each condition. Results were normalized to 786-O only with DMSO treatment.

FIG. 15, comprising FIGS. 15A-15D, depicts the results of experiments demonstrating that tumor associated neutrophils promote RCC invasion in orthotopically implanted RCC model. 786-O cells (9×10⁵) and HL-60N cells (1×10⁵) were orthotopically co-implanted into the renal capsule of nude mice. After 6 weeks, the RCC growth and metastasis were evaluated. FIG. 15A. The 786-O cells co-injected with HL-60N cells showed a higher tumor growth rate. Tumor weight in each group was measured. FIG. 15B. Metastases were found in the diaphragm (middle panel, representative picture) of neutrophils co-implanted RCC tumors. RCC cells were stably transfected with luciferase cDNA and IVIS imaging was applied to monitor the RCC growth and metastasis. Mouse numbers with or without metastatic tumors of each group were quantitated (right panel, n=10 for each group). FIG. 15C. Histology analyses of RCC tumors formed in nude mice. IHC and H&E results indicated that HL-60N co-implanted RCC tumors are positively correlated with higher ERβ (upper right panel), VEGFa (lower left panel) and HIF2α (lower right panel). Anti-ERβ antibody can detect predominate nuclei staining signals of ERβ in RCC tumors. FIG. 15D. Summary of mechanisms and regulatory pathways of neutrophils-promoted RCC progression. RCC cells can better recruit neutrophil infiltration. RCC cells and neutrophils interact with each other in the RCC tumor microenvironment. The infiltrated neutrophils can function via increasing ERβ to up-regulate VEGFa and HIF2α signaling and lead to an increased RCC invasion.

### DETAILED DESCRIPTION

The present invention relates generally to compositions and methods for treating cancer. The invention is useful, for example, for reducing tumor growth and tumor cell proliferation. In certain embodiments, the invention provides for a hormonal approach for the treatment of a cancer. In certain embodiments, the present invention treats or prevents any cancer or tumor associated with overexpression of estrogen receptors. In one embodiment, the present invention treats or prevents any cancer or tumor associated with overexpression of estrogen receptors in males or in both males and females. In one embodiment, the invention relates to the inhibition of estrogen receptors, including estrogen receptor β (ERβ), to treat or prevent renal cell carcinoma (RCC). In one embodiment, the invention relates to methods of using ERβ as a biomarker for RCC progression.

In one embodiment, the present invention is a method of treating cancer associated with elevated levels of ERβ comprising the administration of an inhibitor of ERβ to patient, preferably a male patient. In a preferred embodiment, one would (1) obtain a diagnosis of cancer in a patient by assessing the levels of ERβ in the patient or a biological sample derived from the patient, (2) administer an effective amount of an inhibitor of ERβ, wherein the amount is effective to reduce cancer symptoms, and (3) examine the patient for reduction of cancer symptoms.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

Generally, the nomenclature used herein and the laboratory procedures in cell culture, molecular genetics, organic chemistry, and nucleic acid chemistry and hybridization are those well-known and commonly employed in the art.

Standard techniques are used for nucleic acid and peptide synthesis. The techniques and procedures are generally performed according to conventional methods in the art and various general references (e.g., Sambrook and Russell, 2012, Molecular Cloning, A Laboratory Approach, Cold Spring Harbor Press, Cold Spring Harbor, N.Y., and Ausubel et al., 2012, Current Protocols in Molecular Biology, John Wiley & Sons, NY), which are provided throughout this document.

The nomenclature used herein and the laboratory procedures used in analytical chemistry and organic syntheses described below are those well-known and commonly employed in the art. Standard techniques or modifications thereof are used for chemical syntheses and chemical analyses.

As used herein, each of the following terms has the meaning associated with it in this section.

The articles “a” and “an” are used herein to refer to one or to more than one (i.e., to at least one) of the grammatical object of the article. By way of example, “an element” means one element or more than one element. “About” as used herein when referring to a measurable value such as an amount, a temporal duration, and the like, is meant to encompass variations of ±20%, or ±10%, or ±5%, or ±1%, or ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

The term “activate,” as used herein, means to induce or increase an activity or function, for example, about ten percent relative to a control value. Preferably, the activity is induced or increased by 50% compared to a control value, more preferably by 75%, and even more preferably by 95%. “Activate,” as used herein, also means to increase a molecule, a reaction, an interaction, a gene, an mRNA, and/or a protein's expression, stability, function or activity by a measurable amount or to increase entirely. Activators are compounds that, e.g., bind to, partially or totally induce stimulation, increase, promote, induce activation, activate, sensitize, or up regulate a protein, a gene, and an mRNA stability, expression, function and activity, e.g., agonists.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The an antibody in the present invention may exist in a variety of forms where the antigen binding portion of the antibody is expressed as part of a contiguous polypeptide chain including, for example, a single domain antibody fragment (sdAb), a single chain antibody (scFv) and a humanized antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

The term “antibody fragment” refers to at least one portion of an intact antibody and refers to the antigenic determining variable regions of an intact antibody. Examples of antibody fragments include, but are not limited to, Fab, Fab′, F(ab′)2, and Fv fragments, linear antibodies, sdAb (either V_{L} or V_{H}), camelid V_{HH} domains, scFv antibodies, and multi-specific antibodies formed from antibody fragments. The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it was derived. Unless specified, as used herein an scFv may have the V_{L} and V_{H} variable regions in either order, e.g., with respect to the N-terminal and C-terminal ends of the polypeptide, the scFv may comprise V_{L}-linker-V_{H} or may comprise V_{H}-linker-V_{L}.

An “antibody heavy chain,” as used herein, refers to the larger of the two types of polypeptide chains present in antibody molecules in their naturally occurring conformations, and which normally determines the class to which the antibody belongs.

An “antibody light chain,” as used herein, refers to the smaller of the two types of polypeptide chains present in antibody molecules in their naturally occurring conformations. Kappa (κ) and lambda (λ) light chains refer to the two major antibody light chain isotypes.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art. The term “anti-tumor effect” as used herein, refers to a biological effect which can be manifested by a decrease in tumor volume, a decrease in the number of tumor cells, a decrease in the number of metastases, an increase in life expectancy, or amelioration of various physiological symptoms associated with the cancerous condition.

An “anti-tumor effect” can also be manifested by the ability of the peptides, polynucleotides, cells and antibodies of the invention in prevention of the occurrence of tumor in the first place.

The term “cancer” as used herein is defined as disease characterized by the abnormal growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer, sarcoma and the like.

A “disease” is a state of health of an animal wherein the animal cannot maintain homeostasis, and wherein if the disease is not ameliorated then the animal's health continues to deteriorate.

A “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity or frequency of at least one sign or symptom of the disease or disorder experienced by a patient is reduced.

“Effective amount” or “therapeutically effective amount” are used interchangeably herein, and refer to an amount of a compound, formulation, material, or composition, as described herein effective to achieve a particular biological result. Such results may include, but are not limited to, the inhibition of virus infection as determined by any means suitable in the art.

As used herein “endogenous” refers to any material from or produced inside an organism, cell, tissue or system.

As used herein “estrogen receptor” refers to any protein in the nuclear receptor gene family that binds estrogen, including, but not limited to, any isoforms or deletion mutations having the characteristics just described. More particularly, the present invention relates to estrogen receptor(s) for humans. Human estrogen receptors included in the present invention include the α- and β-isoforms (referred to herein as “ERα” and “ERβ”) in addition to any additional isoforms as recognized by those of skill in the biochemistry arts.

As used herein, the term “exogenous” refers to any material introduced from or produced outside an organism, cell, tissue or system.

The term “expression” as used herein is defined as the transcription and/or translation of a particular nucleotide sequence driven by its promoter.

The term “expression vector” as used herein refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules, siRNA, ribozymes, and the like. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well.

The term “inhibit,” as used herein, means to suppress or block an activity or function, for example, about ten percent relative to a control value. Preferably, the activity is suppressed or blocked by 50% compared to a control value, more preferably by 75%, and even more preferably by 95%. “Inhibit,” as used herein, also means to reduce a molecule, a reaction, an interaction, a gene, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or down regulate a protein, a gene, and an mRNA stability, expression, function and activity, e.g., antagonists.

As used herein, an “instructional material” includes a publication, a recording, a diagram, or any other medium of expression which can be used to communicate the usefulness of the compositions and methods of the invention. The instructional material of the kit of the invention may, for example, be affixed to a container which contains the nucleic acid, peptide, and/or composition of the invention or be shipped together with a container which contains the nucleic acid, peptide, and/or composition. Alternatively, the instructional material may be shipped separately from the container with the intention that the instructional material and the compound be used cooperatively by the recipient.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

“Naturally-occurring” as applied to an object refers to the fact that the object can be found in nature. For example, a polypeptide or polynucleotide sequence that is present in an organism (including viruses) that can be isolated from a source in nature and which has not been intentionally modified by man is a naturally-occurring sequence.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil). The term “nucleic acid” typically refers to large polynucleotides.

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

The term “operably linked” as used herein refer to the linkage of nucleic acid sequences in such a manner that a nucleic acid molecule capable of directing the transcription of a given gene and/or the synthesis of a desired protein molecule is produced. The term also refers to the linkage of sequences encoding amino acids in such a manner that a functional (e.g., enzymatically active, capable of binding to a binding partner, capable of inhibiting, etc.) protein or polypeptide is produced.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in an inducible manner.

An “inducible” promoter is a nucleotide sequence which, when operably linked with a polynucleotide which encodes or specifies a gene product, causes the gene product to be produced substantially only when an inducer which corresponds to the promoter is present.

A “constitutive” promoter is a nucleotide sequence which, when operably linked with a polynucleotide which encodes or specifies a gene product, causes the gene product to be produced in a cell under most or all physiological conditions of the cell.

“Polypeptide” refers to a polymer composed of amino acid residues, related naturally occurring structural variants, and synthetic non-naturally occurring analogs thereof linked via peptide bonds. Synthetic polypeptides can be synthesized, for example, using an automated polypeptide synthesizer.

The term “protein” typically refers to large polypeptides.

The term “peptide” typically refers to short polypeptides.

Conventional notation is used herein to portray polypeptide sequences: the left-hand end of a polypeptide sequence is the amino-terminus; the right-hand end of a polypeptide sequence is the carboxyl-terminus.

As used herein, a “peptidomimetic” is a compound containing non-peptidic structural elements that is capable of mimicking the biological action of a parent peptide. A peptidomimetic may or may not comprise peptide bonds.

A “polynucleotide” means a single strand or parallel and anti-parallel strands of a nucleic acid. Thus, a polynucleotide may be either a single-stranded or a double-stranded nucleic acid. In the context of the present invention, the following abbreviations for the commonly occurring nucleic acid bases are used. “A” refers to adenosine, “C” refers to cytidine, “G” refers to guanosine, “T” refers to thymidine, and “U” refers to uridine.

The term “oligonucleotide” typically refers to short polynucleotides, generally no greater than about 60 nucleotides. It will be understood that when a nucleotide sequence is represented by a DNA sequence (i.e., A, T, G, C), this also includes an RNA sequence (i.e., A, U, G, C) in which “U” replaces “T.”

As used herein, a “recombinant cell” is a host cell that comprises a recombinant polynucleotide.

“Sample” or “biological sample” as used herein means a biological material from a subject, including but is not limited to organ, tissue, exosome, blood, plasma, saliva, urine and other body fluid. A sample can be any source of material obtained from a subject.

By the term “specifically binds,” as used herein, is meant a molecule, such as an antibody, which recognizes and binds to another molecule or feature, but does not substantially recognize or bind other molecules or features in a sample.

The terms “subject,” “patient,” “individual,” and the like are used interchangeably herein, and refer to any animal, or cells thereof whether in vitro or in situ, amenable to the methods described herein. In certain non-limiting embodiments, the patient, subject or individual is a human.

“Therapeutically effective amount” is an amount of a compound of the invention, that when administered to a patient, ameliorates a symptom of the disease. The amount of a compound of the invention which constitutes a “therapeutically effective amount” will vary depending on the compound, the disease state and its severity, the age of the patient to be treated, and the like. The therapeutically effective amount can be determined routinely by one of ordinary skill in the art having regard to his own knowledge and to this disclosure.

The terms “treat,” “treating,” and “treatment,” refer to therapeutic or preventative measures described herein. The methods of “treatment” employ administration to a subject, in need of such treatment, a composition of the present invention, for example, a subject afflicted a disease or disorder, or a subject who ultimately may acquire such a disease or disorder, in order to prevent, cure, delay, reduce the severity of, or ameliorate one or more symptoms of the disorder or recurring disorder, or in order to prolong the survival of a subject beyond that expected in the absence of such treatment.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The invention is based partly on the discovery that ERβ expression increases with the stage and grade rise in RCC. The invention is also based partly on the discovery that ERβ modulates the TGFβ1-SMAD3 pathway in cancer cell invasion. The invention is also based partly on the discovery that ERβ modulates neutrophil-promoted RCC cell migration via induction of the VEGFa-HIF2α pathway.

The present invention relates generally to compositions and methods for treating cancer. The present invention is useful for treating cancer in males or in both males and females. In one embodiment, the present invention is directed to methods and compositions for the treatment, inhibition, prevention, or reduction of a cancer using an inhibitor of ERβ. In one embodiment, the present invention provides a composition for treating, inhibiting, preventing, or reducing RCC in a subject, wherein the composition comprises an inhibitor of ERβ.

While the present invention is exemplified herein by targeting of ERβ, the present invention is not limited to the targeting of this particular receptor. Rather, it is demonstrated herein that targeting additional proteins in relevant pathways may also enhance the treatment effect, such as TGFβ-1, SMAD, progesterone receptor (PR), and HIF. Therefore, the present invention contemplates the targeting of these proteins as well.

Exemplary forms of cancer that are treatable or preventable with the compositions and methods of the present invention include, but are not limited to, renal cell carcinoma, carcinomas, sarcomas, lymphomas, leukemia, blastomas, and germ cell cancers. Other exemplary forms of cancer that are treatable or preventable with the compositions and methods of the present invention include, but are not limited to, breast cancer, lung cancer, pancreatic cancer, stomach cancer, bone cancer, ovarian cancer, prostate cancer, bladder cancer, cervical cancer, colon cancer, skin cancer, gliomas, esophageal cancer, oral cancer, gallbladder cancer, liver cancer, testicular cancer, uterine cancer, thyroid cancer, throat cancer, Li-Fraumeni Syndrome and the like.

In one embodiment, the present invention provides a composition for treating cancer in a subject, preferably a male, wherein the composition comprises an inhibitor of one or more estrogen receptors. In one embodiment, the present invention provides a composition for treating cancer in a subject, wherein the composition comprises an inhibitor of ERβ. In one embodiment, the present invention provides a composition for treating cancer in a subject, wherein the composition comprises an inhibitor of ERβ with an inhibitor of TGFβ-1. In one embodiment, the present invention provides a composition for treating cancer in a subject, wherein the composition comprises an inhibitor of ERβ and an inhibitor of SMAD. In one embodiment, the present invention provides a composition for treating cancer in a subject, wherein the composition comprises an inhibitor of ERβ and an inhibitor of HIF. In one embodiment, the present invention provides a composition for treating cancer in a subject, wherein the composition comprises an inhibitor of ERβ, an inhibitor of TGFβ-1, an inhibitor of SMAD, and an inhibitor of HIF.

### Compositions

In one embodiment, the present invention provides a composition for treating or preventing cancer in a subject, wherein the composition inhibits tumor cell proliferation or reduces tumor growth. In certain embodiments, the composition inhibits the expression, activity, or both of ERβ, TGFβ-1, SMAD, HIF, or a combination thereof in a tumor cell of the subject.

In one embodiment, the composition of the invention comprises an inhibitor of ERβ, an inhibitor of TGFβ-1, an inhibitor of SMAD, an inhibitor of HIF, or a combination thereof. An inhibitor of ERβ, an inhibitor of TGFβ-1, an inhibitor of SMAD, or an inhibitor of HIF is any compound, molecule, or agent that reduces, inhibits, or prevents the function of ERβ, TGFβ-1, SMAD, PR, or HIF. For example, an inhibitor of ERβ, an inhibitor of TGFβ-1, an inhibitor of SMAD, or an inhibitor of HIF is any compound, molecule, or agent that reduces the expression, activity, or both of ERβ, TGFβ-1, SMAD, PR or HIF. In certain embodiments, the inhibitor inhibits the transcription of DNA, inhibits the translation of RNA, or inhibits the protein itself. In one embodiment, an inhibitor of ERβ, an inhibitor of TGFβ-1, an inhibitor of SMAD, or an inhibitor of HIF comprises a nucleic acid, a peptide, an antibody, a small molecule, a siRNA, a ribozyme, an antisense nucleic acid, an antagonist, an aptamer, a peptidomimetic, or any combination thereof.

Small Molecule Inhibitors

In various embodiments, the inhibitor is a small molecule. When the inhibitor is a small molecule, a small molecule may be obtained using standard methods known to the skilled artisan. Such methods include chemical organic synthesis or biological means. Biological means include purification from a biological source, recombinant synthesis and in vitro translation systems, using methods well known in the art. In one embodiment, a small molecule inhibitor of the invention comprises an organic molecule, inorganic molecule, biomolecule, synthetic molecule, and the like.

Combinatorial libraries of molecularly diverse chemical compounds potentially useful in treating a variety of diseases and conditions are well known in the art as are method of making the libraries. The method may use a variety of techniques well-known to the skilled artisan including solid phase synthesis, solution methods, parallel synthesis of single compounds, synthesis of chemical mixtures, rigid core structures, flexible linear sequences, deconvolution strategies, tagging techniques, and generating unbiased molecular landscapes for lead discovery vs. biased structures for lead development.

In a general method for small library synthesis, an activated core molecule is condensed with a number of building blocks, resulting in a combinatorial library of covalently linked, core-building block ensembles. The shape and rigidity of the core determines the orientation of the building blocks in shape space. The libraries can be biased by changing the core, linkage, or building blocks to target a characterized biological structure (“focused libraries”) or synthesized with less structural bias using flexible cores.

Small molecule inhibitors of ERβ suitable for use in the present invention include, but are not limited to, 7α-[9-(4,4,5,5,5-pentafluoropentylsulfinyl)nonyl]estra-1,3,5(10)-triene-3,17β-diol (also known as ICI 182,780), N-n-butyl-N-methyl-11-(3,17β-dihydroxyestra-1,3,5(10)-triene-7α-yl)undecanamide (also known as ICI 164,384), 11β-[4-[2-(dimethylaminoethoxyl]phenyl]-estradiol (also known as RU 39411), N-methyl-N-isopropyl-(3,17β-dihydroxy-estra-1,2,5(10)-trien-11-β-yl)-undecamide (RU 51625), its 17α-ethynyl derivative (RU 53637), [6-hydroxy-2(4-hydroxyphenyl)benzo[b]thien-3-yl][4-[3-(1-pyrrolidin-yl)ethoxy]phenyl]methanone (LY117018), tamoxifen, toremifene, fulvestrant, raloxifene, raloxifene hydrochloride, clomiphene, keoxifene, 3-[4-(1,2-Diphenylbut-1-enyl)phenyl]acrylic acid, and the like, including the active metabolites of these compounds. In one embodiment, the ERβ inhibitor is SR-13688 ((E)-3-hydroxy-21-[2′-(N,N-dimethylamino)ethoxy]-19-norpregna-1,3,5(10),17(20)-tetraene) as described in WO2011133475A2. These compounds may be made in accordance with known procedures which will be apparent to those skilled in the art. See, e.g., A. E. Wakeling et al., Canc. Res. 51, 3867-3873 (1991); D. Poirier et al., J. Med. Chem. 37, 1115-1125 (1994); J. Bowler et al., Steroids 54, 71-79 (1989); C. Levesque et al., J. Med. Chem. 34, 1624-1630 (1991); A. Claussner et al., J. Steroid. Biochem. Mol. Biol. 41, 609-614 (1992).

Further examples of ERβ inhibitors include an oxabicycloheptene or oxabicycloheptadiene bearing a 1,2-diarylehtylene motif (as disclosed in Zhou et al., J. Med. Chem. 48(23):7261 (2005), an oxabicycloheptene sulfonamide (as disclosed in Zhu et al., Org. Biomol. Chem. 10(43):8692 (2012)), a bicyclic pyrazolo[1,5-a]pyrimidine (as disclosed in Compton et al., J. Med. Chem. 48(7):2724 (2005)), a 2,3-diarylpyrazolo[1,5-a]pyrimidine (as disclosed in Zhou et al., J. Med Chem. 50(2):399 (2007)), a diarylpropionitrile (as disclosed in Meyers et al., J. Med Chem. 44:4230 (2001)), 2,3-bis(4-hydroxyphenyl)propanenitrile (disclosed in Meyers et al., J. Med Chem. 44:4230 (2001)), 2-phenyl-3-(4-hydroxyphenyl)-5,7-bis(trifluoromethyl)-pyrazolo[1,5-a]pyrimidine (disclosed in Compton et al., J. Med. Chem. 48(7):2724 (2005)), and Faslodex (7α,17β-[9-[(4,4,5,5,5-Pentafluoropentyl)sulfinyl]nonyl]estra-1,3,5(10)-triene-3,17-diol).

Potentially useful anti-estrogens include aromatase inhibitors such as Letrozole (4-[(4-cyanophenyl)-(1,2,4-triazol-1-yl)methyl]benzonitrile), Anastrozole (2-[3-(2-cyanopropan-2-yl)-5-(1,2,4-triazol-1-ylmethyl)phenyl]-2-methylpropanenitrile), Vorozole (6-[(4-chlorophenyl)-(1,2,4-triazol-1-yl)methyl]-1-methylbenzotriazole), Exemestane ((8R,9S,10R,13S,14S)-10,13-dimethyl-6-methylidene-7,8,9,11,12,14,15,16-octahydrocyclopenta[a]phenanthrene-3,17-dione), Foremestane ((8R,9S,10R,13S,14S)-4-hydroxy-10,13-dimethyl-2,6,7,8,9,11,12,14,15,16-decahydro-1H-cyclopenta[a]phenanthrene-3,17-dione), and Atemestane ((8R,9S,10S,13S,14S)-1,10,13-trimethyl-7,8,9,11,12,14,15,16-octahydro-6H-cyclopenta[a]phenanthrene-3,17-dione)).

In one embodiment, the ERβ inhibitor is an inverse agonist to ERβ. In another embodiment, the ERβ inhibitor is an antagonist to ERβ. In another embodiment, the ERβ inhibitor is a selective estrogen receptor modulator (SERM), a steroidal aromatase inhibitor (e.g., exemestane), or a non-steroidal aromatase inhibitor (e.g., anastrozole).

A variety of TGF-β inhibitors and methods for their production are well known in the art and many more are currently under development. The specific TGF-β antagonist employed is not a limiting feature, as any effective TGF-β antagonist may be useful in the methods of this invention. One particular TGF-β antagonist that is useful in the methods of the invention is 2-butyl-4-chloro-1-[p-(o-1H-tetrazol-5-ylphenyl)benzyl]imidazole-5-methanol monopotassium salt (losartan potassium). Further examples of such antagonists include monoclonal and polyclonal antibodies directed against one or more isoforms of TGF-β (U.S. Pat. No. 5,571,714 and PCT patent application WO 97/13844), TGF-β receptors, fragments thereof, derivatives thereof and antibodies directed against TGF-β receptors (U.S. Pat. Nos. 5,693,607, 6,008,011, 6,001,969 and 6,010,872 and PCT patent applications WO 92/00330, WO 93/09228, WO 95/10610 and WO 98/48024); latency associated peptide (WO 91/08291), large latent TGF-β (WO 94/09812), fetuin (U.S. Pat. No. 5,821,227), decorin and other proteoglycans such as biglycan, fibromodulin, lumican and endoglin (U.S. Pat. Nos. 5,583,103, 5,654,270, 5,705,609, 5,726,149, 5,824,655 5,830,847, 6,015,693 and PCT patent applications WO 91/04748, WO 91/10727, WO 93/09800 and WO 94/10187).

Further examples of such inhibitors include LY2157299 H₂O (Bueno et al., Eur. J. Cancer 44(1):142 (2008)), GW788388 (Petersen et al., Kidney Int. 73(6):705 (2007)), SB-431542 (Inman et al., Mol. Pharmacol. 62(10):65 (2002)), the dihydropyrrolopyrazole compounds LY550410 and LY580276 disclosed in Yingling et al., Nat. Review 3:1011 at Tbl. 3 (2004), the imidazole compound SB-505124 disclosed in Yingling et al., Nat. Review 3:1011 at Tbl. 3 (2004), the pyrazolopyridine (Lilly Research Laboratories compound 1) disclosed in Yingling et al., Nat. Review 3:1011 at Tbl. 3 (2004), the pyrazole (GSK compound 2) disclosed in Yingling et al., Nat. Review 3:1011 at Tbl. 3 (2004), the imidazopyridine (Biogen Idec compound 3) disclosed in Yingling et al., Nat. Review 3:1011 at Tbl. 3 (2004), the triazole (Pfizer compound 4) disclosed in Yingling et al., Nat. Review 3:1011 at Tbl. 3 (2004), the pyridopyrimidine (J&J/Scios compound 5) disclosed in Yingling et al., Nat. Review 3:1011 at Tbl. 3 (2004), the isothiazole (Pfizer compound 6) disclosed in Yingling et al., Nat. Review 3:1011 at Tbl. 3 (2004), or neutralizing antibodies 2G7, 1D11, or GC1008 (Arteaga et al., J Clin Invest 92: 2569 (1993); Pinkas et al., Biochem Pharmacol 72:523 (2006); Nam et al., Cancer Res 66:6327 (2006); Ananth et al., Cancer Res 59:2210 (1999); Tan et al., Breast Cancer Res Treat 115:453 (2009)) the antibodies disclosed in Yingling et al., Nat. Review 3:1011 at Tbl. 1 (2004), somatostatin (PCT patent application WO 98/08529), mannose-6-phosphate or mannose-1-phosphate (U.S. Pat. No. 5,520,926), prolactin (PCT patent application WO 97/40848), insulin-like growth factor II (PCT patent application WO 98/17304), IP-10 (PCT patent application WO97/00691), arg-gly-asp containing peptides (U.S. Pat. No. 5,958,411 and PCT patent application WO 93/10808 and), extracts of plants, fungi and bacteria (European patent application 813875, Japanese patent application 8119984 and U.S. Pat. No. 5,693,610), antisense oligonucleotides (U.S. Pat. Nos. 5,683,988, 5,772,995, 5,821,234 and 5,869,462 and PCT patent application WO 94/25588), and a host of other proteins involved in TGF-β signaling, including SMADs and MADs (European patent application EP 874046, PCT patent applications WO 97/31020, WO 97/38729, WO 98/03663, WO 98/07735, WO 98/07849, WO 98/45467, WO 98/53068, WO 98/55512, WO 98/56913, WO 98/53830, and WO 99/50296, and U.S. Pat. Nos. 5,834,248, 5,807,708 and 5,948,639) and Ski and Sno (G. Vogel, Science, 286:665 (1999) and Stroschein et al., Science, 286:771-74 (1999)) and fragments and derivatives of any of the above molecules that retain the ability to inhibit the activity of TGF-β. SMAD inhibitors include 6,7-Dimethoxy-2-((2E)-3-(1-methyl-2-phenyl-1H-pyrrolo[2,3-b]pyridin-3-yl-prop-2-enoyl))-1,2,3,4-tetrahydroisoquinoline.

Inhibitors of neutrophils include HIF inhibitors and rapamycin. The HIF inhibitor may be echinomycin, 2-methoxyestradiol, or geldanamycin. The echinomycin may be a peptide antibiotic such as N,N′-(2,4,12,15,17,25-hexamethyl-11,24-bis(1-methylethyl)-27-(methylthio)-3,6,10,13,16,19,23,26-octaoxo-9,22-dioxa-28-thia-2,5,12,15,18,25-hexaazabicyclo(12.12.3)nonacosane-7,20-diyl)bis(2-quinoxalinecarboxamide). The echinomycin may be a microbially-derived quinoxaline antibiotic, which may be produced by Streptomyces echinatus. The echinomycin may also be an echinomycin derivative, which may comprise a modification as described in Gauvreau et al., Can J Microbiol, 1984; 30(6):730-8; Baily et al., Anticancer Drug Des 1999; 14(3):291-303; or Park and Kim, Bioorganic & Medicinal Chemistry Letters, 1998; 8(7):731-4, the contents of which are incorporated by reference. The echinomycin may also be a bis-quinoxaline analog of echinomycin.

The small molecule and small molecule compounds described herein may be present as salts even if salts are not depicted and it is understood that the invention embraces all salts and solvates of the inhibitors depicted here, as well as the non-salt and non-solvate form of the inhibitors, as is well understood by the skilled artisan. In some embodiments, the salts of the inhibitors of the invention are pharmaceutically acceptable salts.

Where tautomeric forms may be present for any of the inhibitors described herein, each and every tautomeric form is intended to be included in the present invention, even though only one or some of the tautomeric forms may be explicitly depicted. For example, when a 2-hydroxypyridyl moiety is depicted, the corresponding 2-pyridone tautomer is also intended.

The invention also includes any or all of the stereochemical forms, including any enantiomeric or diasteriomeric forms of the inhibitors described. The recitation of the structure or name herein is intended to embrace all possible stereoisomers of inhibitors depicted. All forms of the inhibitors are also embraced by the invention, such as crystalline or non-crystalline forms of the inhibitors. Compositions comprising an inhibitor of the invention are also intended, such as a composition of substantially pure inhibitor, including a specific stereochemical form thereof, or a composition comprising mixtures of inhibitors of the invention in any ratio, including two or more stereochemical forms, such as in a racemic or non-racemic mixture.

In one embodiment, the small molecule inhibitor of the invention comprises an analog or derivative of an inhibitor described herein.

In one embodiment, the small molecules described herein are candidates for derivatization. As such, in certain instances, the analogs of the small molecules described herein that have modulated potency, selectivity, and solubility are included herein and provide useful leads for drug discovery and drug development. Thus, in certain instances, during optimization new analogs are designed considering issues of drug delivery, metabolism, novelty, and safety.

In some instances, small molecule inhibitors described herein are derivatized/analoged as is well known in the art of combinatorial and medicinal chemistry. The analogs or derivatives can be prepared by adding and/or substituting functional groups at various locations. As such, the small molecules described herein can be converted into derivatives/analogs using well known chemical synthesis procedures. For example, all of the hydrogen atoms or substituents can be selectively modified to generate new analogs. Also, the linking atoms or groups can be modified into longer or shorter linkers with carbon backbones or hetero atoms. Also, the ring groups can be changed so as to have a different number of atoms in the ring and/or to include hetero atoms. Moreover, aromatics can be converted to cyclic rings, and vice versa. For example, the rings may be from 5-7 atoms, and may be homocycles or heterocycles.

As used herein, the term “analog,” “analogue,” or “derivative” is meant to refer to a chemical compound or molecule made from a parent compound or molecule by one or more chemical reactions. As such, an analog can be a structure having a structure similar to that of the small molecule inhibitors described herein or can be based on a scaffold of a small molecule inhibitor described herein, but differing from it in respect to certain components or structural makeup, which may have a similar or opposite action metabolically. An analog or derivative of any of a small molecule inhibitor in accordance with the present invention can be used to reduce skin pigmentation.

In one embodiment, the small molecule inhibitors described herein can independently be derivatized/analoged by modifying hydrogen groups independently from each other into other substituents. That is, each atom on each molecule can be independently modified with respect to the other atoms on the same molecule. Any traditional modification for producing a derivative/analog can be used. For example, the atoms and substituents can be independently comprised of hydrogen, an alkyl, aliphatic, straight chain aliphatic, aliphatic having a chain hetero atom, branched aliphatic, substituted aliphatic, cyclic aliphatic, heterocyclic aliphatic having one or more hetero atoms, aromatic, heteroaromatic, polyaromatic, polyamino acids, peptides, polypeptides, combinations thereof, halogens, halo-substituted aliphatics, and the like. Additionally, any ring group on a compound can be derivatized to increase and/or decrease ring size as well as change the backbone atoms to carbon atoms or hetero atoms.

MicroRNA

In other related aspects, the invention includes an isolated nucleic acid. In some instances the inhibitor is an siRNA or antisense molecule, which inhibits ERβ, TGFβ-1, SMAD, PR, or HIF. In one embodiment, the nucleic acid comprises a promoter/regulatory sequence such that the nucleic acid is preferably capable of directing expression of the nucleic acid. Thus, the invention encompasses expression vectors and methods for the introduction of exogenous DNA into cells with concomitant expression of the exogenous DNA in the cells such as those described, for example, in Sambrook et al. (2012, Molecular Cloning: A Laboratory Manual, Cold Spring Harbor Laboratory, New York), and in Ausubel et al. (1997, Current Protocols in Molecular Biology, John Wiley & Sons, New York) and as described elsewhere herein.

In one embodiment, siRNA is used to decrease the level of ERβ, TGFβ-1, SMAD, or HIF. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559; Fire et al., 1998, Nature 391(19):306-311; Timmons et al., 1998, Nature 395:854; Montgomery et al., 1998, TIG 14 (7):255-258; David R. Engelke, Ed., RNA Interference (RNAi) Nuts & Bolts of RNAi Technology, DNA Press, Eagleville, P A (2003); and Gregory J. Hannon, Ed., RNAi A Guide to Gene Silencing, Cold Spring Harbor Laboratory Press, Cold Spring Harbor, N.Y. (2003). Soutschek et al. (2004, Nature 432:173-178) describe a chemical modification to siRNAs that aids in intravenous systemic delivery. Optimizing siRNAs involves consideration of overall G/C content, C/T content at the termini, Tm and the nucleotide content of the 3′ overhang. See, for instance, Schwartz et al., 2003, Cell, 115:199-208 and Khvorova et al., 2003, Cell 115:209-216. Therefore, the present invention also includes methods of decreasing levels of ERβ, TGFβ-1, SMAD, or HIF using RNAi technology.

In certain embodiments, the expression vectors described herein encode a short hairpin RNA (shRNA) inhibitor. shRNA inhibitors are well known in the art and are directed against the mRNA of a target, thereby decreasing the expression of the target. In certain embodiments, the encoded shRNA is expressed by a cell, and is then processed into siRNA. For example, in certain instances, the cell possesses native enzymes (e.g., dicer) that cleaves the shRNA to form siRNA.

The siRNA, shRNA, or antisense polynucleotide can be cloned into a number of types of vectors as described elsewhere herein. For expression of the siRNA or antisense polynucleotide, at least one module in each promoter functions to position the start site for RNA synthesis.

In order to assess the expression of the siRNA, shRNA, or antisense polynucleotide, the expression vector to be introduced into a cell can also contain either a selectable marker gene or a reporter gene or both to facilitate identification and selection of expressing cells from the population of cells sought to be transfected or infected using a viral vector. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neomycin resistance and the like.

In one embodiment of the invention, an antisense nucleic acid sequence which is expressed by a plasmid vector is used to inhibit ERβ, TGFβ-1, SMAD, or HIF protein expression. The antisense expressing vector is used to transfect a mammalian cell or the mammal itself, thereby causing reduced endogenous expression of ERβ, TGFβ-1, SMAD, PR, or HIF.

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue, 1993, U.S. Pat. No. 5,190,931.

Alternatively, antisense molecules of the invention may be made synthetically and then provided to the cell. Antisense oligomers of between about 10 to about 30, and more preferably about 15 nucleotides, are preferred, since they are easily synthesized and introduced into a target cell. Synthetic antisense molecules contemplated by the invention include oligonucleotide derivatives known in the art which have improved biological activity compared to unmodified oligonucleotides (see U.S. Pat. No. 5,023,243).

In one embodiment of the invention, a ribozyme is used to inhibit ERβ, TGFβ-1, SMAD, PR, or HIF protein expression. Ribozymes useful for inhibiting the expression of a target molecule may be designed by incorporating target sequences into the basic ribozyme structure which are complementary, for example, to the mRNA sequence encoding ERβ, TGFβ-1, SMAD, PR, or HIF. Ribozymes targeting ERβ, TGFβ-1, SMAD, or HIF, may be synthesized using commercially available reagents (Applied Biosystems, Inc., Foster City, Calif.) or they may be genetically expressed from DNA encoding them.

In one embodiment, the nucleic acid inhibitor of the invention is an antagonist of ERβ, TGFβ-1, SMAD, PR, or HIF. For example, in certain embodiments, the isolated nucleic acid specifically binds to ERβ, TGFβ-1, SMAD, or HIF, or a target of ERβ, TGFβ-1, SMAD, PR, or HIF, to inhibit the functional activity of ERβ, TGFβ-1, SMAD, or HIF.

Aptamers

In one embodiment, the composition comprises an aptamer, including for example a protein aptamer or a polynucleotidal aptamer. In one embodiment, the aptamer inhibits the expression, activity, or both of ERβ, TGFβ-1, SMAD, PR, or HIF.

In one embodiment, an apatmer is a nucleic acid or oligonucleotide molecule that binds to a specific molecular target, such as ERβ, TGFβ-1, SMAD, PR, or HIF. In one embodiment, aptamers are obtained from an in vitro evolutionary process known as SELEX (Systematic Evolution of Ligands by EXponential Enrichment), which selects target-specific aptamer sequences from combinatorial libraries of single stranded oligonucleotide templates comprising randomized sequences. In some embodiments, aptamer compositions are double-stranded or single-stranded, and in various embodiments include deoxyribonucleotides, ribonucleotides, nucleotide derivatives, or other nucleotide-like molecules. In some embodiments, the nucleotide components of an aptamer include modified or non-natural nucleotides, for example nucleotides that have modified sugar groups (e.g., the 2′-OH group of a ribonucleotide is replaced by 2′-F or 2′-NH₂), which in some instances, improves a desired property, e.g., resistance to nucleases or longer lifetime in blood.

In some instances, individual aptamers having the same nucleotide sequence differ in their secondary structure. In some embodiments, the aptamers of the invention are conjugated to other molecules, e.g., a high molecular weight carrier to slow clearance of the aptamer from the circulatory system. In some instances, aptamers are specifically cross-linked to their cognate ligands, e.g., by photo-activation of a cross-linker. (Brody, E. N. and L. Gold (2000) J. Biotechnol. 74:5-13).

Peptide Inhibitors

In other related aspects, the invention includes an isolated peptide inhibitor that inhibits ERβ, TGFβ-1, SMAD, PR, or HIF. For example, in one embodiment, the peptide inhibitor of the invention inhibits ERβ, TGFβ-1, SMAD, PR, or HIF directly by binding to ERβ, TGFβ-1, SMAD, PR, or HIF thereby preventing the normal functional activity of ERβ, TGFβ-1, SMAD, PR, or HIF. In another embodiment, the peptide inhibitor of the invention inhibits ERβ, TGFβ-1, SMAD, PR, or HIF by competing with endogenous ERβ, TGFβ-1, SMAD, PR, or HIF. In yet another embodiment, the peptide inhibitor of the invention inhibits the activity of ERβ, TGFβ-1, SMAD, PR, or HIF by acting as a transdominant negative mutant.

The variants of the polypeptides according to the present invention may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the polypeptide is an alternative splice variant of the polypeptide of the present invention, (iv) fragments of the polypeptides and/or (v) one in which the polypeptide is fused with another polypeptide, such as a leader or secretory sequence or a sequence which is employed for purification (for example, His-tag) or for detection (for example, Sv5 epitope tag). The fragments include polypeptides generated via proteolytic cleavage (including multi-site proteolysis) of an original sequence. Variants may be post-translationally, or chemically modified. Such variants are deemed to be within the scope of those skilled in the art from the teaching herein.

As known in the art the “similarity” between two polypeptides is determined by comparing the amino acid sequence and its conserved amino acid substitutes of one polypeptide to a sequence of a second polypeptide. Variants are defined to include polypeptide sequences different from the original sequence, preferably different from the original sequence in less than 40% of residues per segment of interest, more preferably different from the original sequence in less than 25% of residues per segment of interest, more preferably different by less than 10% of residues per segment of interest, most preferably different from the original protein sequence in just a few residues per segment of interest and at the same time sufficiently homologous to the original sequence to preserve the functionality of the original sequence and/or the ability to bind to ubiquitin or to a ubiquitylated protein. The present invention includes amino acid sequences that are at least 60%, 65%, 70%, 72%, 74%, 76%, 78%, 80%, 90%, or 95% similar or identical to the original amino acid sequence. The degree of identity between two polypeptides is determined using computer algorithms and methods that are widely known for the persons skilled in the art. The identity between two amino acid sequences is preferably determined by using the BLASTP algorithm [BLAST Manual, Altschul, S., et al., NCBI NLM NIH Bethesda, Md. 20894, Altschul, S., et al., J. Mol. Biol. 215: 403-410 (1990)].

The polypeptides of the invention can be post-translationally modified. For example, post-translational modifications that fall within the scope of the present invention include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction.

In one embodiment, the composition comprises a peptidomimetic inhibitor of at least one of ERβ, TGFβ-1, SMAD, PR, or HIF. Peptidomimetics are compounds based on, or derived from, peptides and proteins. The peptidomimetics of the present invention typically can be obtained by structural modification of known ERβ, TGFβ-1, SMAD, PR, or HIF sequences or sequences that interact with ERβ, TGFβ-1, SMAD, PR, or HIF, using unnatural amino acids, conformational restraints, isosteric replacement, and the like. The peptidomimetics constitute the continum of structural space between peptides and non-peptide synthetic structures.

Such peptidomimetics can have such attributes as being non-hydrolyzable (e.g., increased stability against proteases or other physiological conditions which degrade the corresponding peptide), increased specificity and/or potency, and increased cell permeability for intracellular localization of the peptidomimetic. For illustrative purposes, peptide analogs of the present invention can be generated using, for example, benzodiazepines (e.g., see Freidinger et al. in Peptides: Chemistry and Biology, G. R. Marshall ed., ESCOM Publisher: Leiden, Netherlands, 1988), substituted gamma lactam rings (Garvey et al. in Peptides: Chemistry and Biology, G. R. Marshall ed., ESCOM Publisher: Leiden, Netherlands, 1988, p 123), C-7 mimics (Huffman et al. in Peptides: Chemistry and Biology, G. R. Marshall ed., ESCOM Publisher: Leiden, Netherlands, 1988, p. 105), keto-methylene pseudopeptides (Ewenson et al. (1986) J Med Chem 29:295; and Ewenson et al. in Peptides: Structure and Function (Proceedings of the 9th American Peptide Symposium) Pierce Chemical Co. Rockland, Ill., 1985), β-turn dipeptide cores (Nagai et al. (1985) Tetrahedron Lett 26:647; and Sato et al. (1986) J Chem Soc Perkin Trans 1:1231), β-aminoalcohols (Gordon et al. (1985) Biochem Biophys Res Commun 126:419; and Dann et al. (1986) Biochem Biophys Res Commun 134:71), diaminoketones (Natarajan et al. (1984) Biochem Biophys Res Commun 124:141), and methyleneamino-modified (Roark et al. in Peptides: Chemistry and Biology, G. R. Marshall ed., ESCOM Publisher: Leiden, Netherlands, 1988, p 134). Also, see generally, Session III: Analytic and synthetic methods, in Peptides: Chemistry and Biology, G. R. Marshall ed., ESCOM Publisher: Leiden, Netherlands, 1988)

A peptide or peptidomimetic inhibitor of the invention may be synthesized by conventional techniques. For example, the peptide or peptidomimetic inhibitor may be synthesized by chemical synthesis using solid phase peptide synthesis. These methods employ either solid or solution phase synthesis methods (see for example, J. M. Stewart, and J. D. Young, Solid Phase Peptide Synthesis, 2^{nd} Ed., Pierce Chemical Co., Rockford Ill. (1984) and G. Barany and R. B. Merrifield, The Peptides: Analysis Synthesis, Biology editors E. Gross and J. Meienhofer Vol. 2 Academic Press, New York, 1980, pp. 3-254 for solid phase synthesis techniques; and M Bodansky, Principles of Peptide Synthesis, Springer-Verlag, Berlin 1984, and E. Gross and J. Meienhofer, Eds., The Peptides: Analysis, Synthesis, Biology, suprs, Vol 1, for classical solution synthesis.)

Peptides of the invention may be developed using a biological expression system. The use of these systems allows the production of large libraries of random peptide sequences and the screening of these libraries for peptide sequences that bind to particular proteins. Libraries may be produced by cloning synthetic DNA that encodes random peptide sequences into appropriate expression vectors. (see Christian et al 1992, J. Mol. Biol. 227:711; Devlin et al, 1990 Science 249:404; Cwirla et al 1990, Proc. Natl. Acad, Sci. USA, 87:6378). Libraries may also be constructed by concurrent synthesis of overlapping peptides (see U.S. Pat. No. 4,708,871).

Antibody Inhibitors

The invention also contemplates an inhibitor of ERβ, an inhibitor of TGFβ-1, an inhibitor of SMAD, an inhibitor of PR, or an inhibitor of HIF comprising an antibody, or antibody fragment, specific for ERβ, TGFβ-1, SMAD, PR, or HIF. That is, the antibody can inhibit ERβ, TGFβ-1, SMAD, PR, or HIF to provide a beneficial effect.

The antibodies may be intact monoclonal or polyclonal antibodies, and immunologically active fragments (e.g., a Fab or (Fab)₂ fragment), an antibody heavy chain, an antibody light chain, humanized antibodies, a genetically engineered single chain Fᵥ molecule (Ladner et al, U.S. Pat. No. 4,946,778), or a chimeric antibody, for example, an antibody which contains the binding specificity of a murine antibody, but in which the remaining portions are of human origin. Antibodies including monoclonal and polyclonal antibodies, fragments and chimeras, may be prepared using methods known to those skilled in the art.

Antibodies can be prepared using intact polypeptides or fragments containing an immunizing antigen of interest. The polypeptide or oligopeptide used to immunize an animal may be obtained from the translation of RNA or synthesized chemically and can be conjugated to a carrier protein, if desired. Suitable carriers that may be chemically coupled to peptides include bovine serum albumin and thyroglobulin, keyhole limpet hemocyanin. The coupled polypeptide may then be used to immunize the animal (e.g., a mouse, a rat, or a rabbit).

Polyclonal antibodies useful in the present invention are generated by immunizing rabbits according to standard immunological techniques well-known in the art (see, e.g., Harlow et al., 1988, In: Antibodies, A Laboratory Manual, Cold Spring Harbor, N.Y.). Such techniques include immunizing an animal with a chimeric protein comprising a portion of another protein such as a maltose binding protein or glutathione (GSH) tag polypeptide portion, and/or a moiety such that the antigenic protein of interest is rendered immunogenic (e.g., an antigen of interest conjugated with keyhole limpet hemocyanin, KLH) and a portion comprising the respective antigenic protein amino acid residues. The chimeric proteins are produced by cloning the appropriate nucleic acids encoding the marker protein into a plasmid vector suitable for this purpose, such as but not limited to, pMAL-2 or pCMX.

However, the invention should not be construed as being limited solely to methods and compositions including these antibodies or to these portions of the antigens. Rather, the invention should be construed to include other antibodies, as that term is defined elsewhere herein, to antigens, or portions thereof. Further, the present invention should be construed to encompass antibodies, inter alia, bind to the specific antigens of interest, and they are able to bind the antigen present on Western blots, in solution in enzyme linked immunoassays, in fluorescence activated cells sorting (FACS) assays, in magenetic-actived cell sorting (MACS) assays, and in immunofluorescence microscopy of a cell transiently transfected with a nucleic acid encoding at least a portion of the antigenic protein, for example.

Monoclonal antibodies directed against full length or peptide fragments of a protein or peptide may be prepared using any well-known monoclonal antibody preparation procedures, such as those described, for example, in Harlow et al. (1988, In: Antibodies, A Laboratory Manual, Cold Spring Harbor, N.Y.) and in Tuszynski et al. (1988, Blood, 72:109-115). Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein.

Nucleic acid encoding the monoclonal antibody obtained using the procedures described herein may be cloned and sequenced using technology which is available in the art, and is described, for example, in Wright et al. (1992, Critical Rev. Immunol. 12:125-168), and the references cited therein. Further, the antibody of the invention may be “humanized” using the technology described in, for example, Wright et al., and in the references cited therein, and in Gu et al. (1997, Thrombosis and Hematocyst 77:755-759), and other methods of humanizing antibodies well-known in the art or to be developed.

The present invention also includes the use of humanized antibodies specifically reactive with epitopes of an antigen of interest. The humanized antibodies of the invention have a human framework and have one or more complementarity determining regions (CDRs) from an antibody, typically a mouse antibody, specifically reactive with an antigen of interest. When the antibody used in the invention is humanized, the antibody may be generated as described in Queen, et al. (U.S. Pat. No. 6,180,370), Wright et al., (supra) and in the references cited therein, or in Gu et al. (1997, Thrombosis and Hematocyst 77(4):755-759). The method disclosed in Queen et al. is directed in part toward designing humanized immunoglobulins that are produced by expressing recombinant DNA segments encoding the heavy and light chain complementarity determining regions (CDRs) from a donor immunoglobulin capable of binding to a desired antigen, such as an epitope on an antigen of interest, attached to DNA segments encoding acceptor human framework regions. Generally speaking, the invention in the Queen patent has applicability toward the design of substantially any humanized immunoglobulin. Queen explains that the DNA segments will typically include an expression control DNA sequence operably linked to the humanized immunoglobulin coding sequences, including naturally-associated or heterologous promoter regions. The expression control sequences can be eukaryotic promoter systems in vectors capable of transforming or transfecting eukaryotic host cells or the expression control sequences can be prokaryotic promoter systems in vectors capable of transforming or transfecting prokaryotic host cells. Once the vector has been incorporated into the appropriate host, the host is maintained under conditions suitable for high level expression of the introduced nucleotide sequences and as desired the collection and purification of the humanized light chains, heavy chains, light/heavy chain dimers or intact antibodies, binding fragments or other immunoglobulin forms may follow (Beychok, Cells of Immunoglobulin Synthesis, Academic Press, New York, (1979), which is incorporated herein by reference).

The invention also includes functional equivalents of the antibodies described herein. Functional equivalents have binding characteristics comparable to those of the antibodies, and include, for example, hybridized and single chain antibodies, as well as fragments thereof. Methods of producing such functional equivalents are disclosed in PCT Application WO 93/21319 and PCT Application WO 89/09622.

Single chain antibodies (scFv) or Fv fragments are polypeptides that consist of the variable region of the heavy chain of the antibody linked to the variable region of the light chain, with or without an interconnecting linker. Thus, the Fv comprises an antibody combining site.

Functional equivalents of the antibodies of the invention further include fragments of antibodies that have the same, or substantially the same, binding characteristics to those of the whole antibody. Such fragments may contain one or both Fab fragments or the F(ab′)₂ fragment. The antibody fragments contain all six complement determining regions of the whole antibody, although fragments containing fewer than all of such regions, such as three, four or five complement determining regions, are also functional. The functional equivalents are members of the IgG immunoglobulin class and subclasses thereof, but may be or may combine with any one of the following immunoglobulin classes: IgM, IgA, IgD, or IgE, and subclasses thereof. Heavy chains of various subclasses, such as the IgG subclasses, are responsible for different effector functions and thus, by choosing the desired heavy chain constant region, hybrid antibodies with desired effector function are produced. Exemplary constant regions are gamma 1 (IgG1), gamma 2 (IgG2), gamma 3 (IgG3), and gamma 4 (IgG4). The light chain constant region can be of the kappa or lambda type.

The immunoglobulins of the present invention can be monovalent, divalent or polyvalent. Monovalent immunoglobulins are dimers (HL) formed of a hybrid heavy chain associated through disulfide bridges with a hybrid light chain. Divalent immunoglobulins are tetramers (H₂L₂) formed of two dimers associated through at least one disulfide bridge.

### Diagnostic Methods

The invention provides methods of diagnosing a cancer in an individual. In one embodiment, the invention provides methods of diagnosing renal cancer in an individual. In one embodiment, the invention provides methods of diagnosing renal cancer in an individual by determining the level of ERβ expression in an individual.

In one embodiment, the invention provides a method of diagnosing an individual as having an ERβ-positive renal cancer, wherein the method comprises obtaining a sample from the individual and determining whether renal cancer cells in the sample are positive for expression of the ERβ. In another embodiment, the method determines that the ERβ expression level in the renal cancer cells of the sample is elevated. In another embodiment, the renal cancer diagnosis includes assessing whether the renal cancer is stage I, stage II, stage III, or stage IV.

The invention also relates to methods of identifying an ERβ-positive cancer in a sample using a reagent, wherein the reagent binds specifically to ERβ; and one or both of the frequency of cells labeled by the reagent and the degree of cell labeling by the reagent are measured. The reagent may be an antibody, antibody fragment, antibody mimic, or nucleic acid aptamer. The sample may be a fixed tissue sample or obtained by biopsy. In one embodiment, the sample is obtained from renal tissue or tissue having metastatic involvement.

The invention also relates to a method for identifying an individual predisposed to development of renal cancer, the method comprising introducing into an individual a reagent that binds specifically to ERβ and can be observed via non-invasive imaging, obtaining an image of the kidneys labeled with reagent; and determining whether renal cells of the kidneys exhibit lower than normal expression levels of ERβ.

### Treatment Methods

The invention provides methods of treating or preventing cancer, or of treating and preventing metastasis of tumors. Related aspects of the invention provide methods of preventing, aiding in the prevention, and/or reducing metastasis of hyperplastic or tumor cells in an individual.

One aspect of the invention provides a method of treating or preventing cancer in an individual in need thereof, the method comprising administering to the individual an effective amount of an inhibitor of ERβ to the individual.

In one embodiment, the invention provides a method to treat cancer comprising treating the subject prior to, concurrently with, or subsequently to the treatment with an inhibitor of ERβ of the invention, with a complementary therapy for the cancer, such as surgery, chemotherapy, chemotherapeutic agent, radiation therapy, or hormonal therapy or a combination thereof.

In another embodiment, the invention provides a method to treat cancer metastasis comprising treating the subject prior to, concurrently with, or subsequently to the treatment with minoxidil or a salt or chemical analog thereof, with a complementary therapy for the cancer, such as surgery, chemotherapy, chemotherapeutic agent, radiation therapy, or hormonal therapy or a combination thereof.

Chemotherapeutic agents include cytotoxic agents (e.g., 5-fluorouracil, cisplatin, carboplatin, methotrexate, daunorubicin, doxorubicin, vincristine, vinblastine, oxorubicin, carmustine (BCNU), lomustine (CCNU), cytarabine USP, cyclophosphamide, estramucine phosphate sodium, altretamine, hydroxyurea, ifosfamide, procarbazine, mitomycin, busulfan, cyclophosphamide, mitoxantrone, carboplatin, cisplatin, interferon alfa-2a recombinant, paclitaxel, teniposide, and streptozoci), cytotoxic alkylating agents (e.g., busulfan, chlorambucil, cyclophosphamide, melphalan, or ethylesulfonic acid), alkylating agents (e.g., asaley, AZQ, BCNU, busulfan, bisulphan, carboxyphthalatoplatinum, CBDCA, CCNU, CHIP, chlorambucil, chlorozotocin, cis-platinum, clomesone, cyanomorpholinodoxorubicin, cyclodisone, cyclophosphamide, dianhydrogalactitol, fluorodopan, hepsulfam, hycanthone, iphosphamide, melphalan, methyl CCNU, mitomycin C, mitozolamide, nitrogen mustard, PCNU, piperazine, piperazinedione, pipobroman, porfiromycin, spirohydantoin mustard, streptozotocin, teroxirone, tetraplatin, thiotepa, triethylenemelamine, uracil nitrogen mustard, and Yoshi-864), antimitotic agents (e.g., allocolchicine, Halichondrin M, colchicine, colchicine derivatives, dolastatin 10, maytansine, rhizoxin, paclitaxel derivatives, paclitaxel, thiocolchicine, trityl cysteine, vinblastine sulfate, and vincristine sulfate), plant alkaloids (e.g., actinomycin D, bleomycin, L-asparaginase, idarubicin, vinblastine sulfate, vincristine sulfate, mitramycin, mitomycin, daunorubicin, VP-16-213, VM-26, navelbine and taxotere), biologicals (e.g., alpha interferon, BCG, G-CSF, GM-CSF, and interleukin-2), topoisomerase I inhibitors (e.g., camptothecin, camptothecin derivatives, and morpholinodoxorubicin), topoisomerase II inhibitors (e.g., mitoxantron, amonafide, m-AMSA, anthrapyrazole derivatives, pyrazoloacridine, bisantrene HCL, daunorubicin, deoxydoxorubicin, menogaril, N,N-dibenzyl daunomycin, oxanthrazole, rubidazone, VM-26 and VP-16), and synthetics (e.g., hydroxyurea, procarbazine, o,p′-DDD, dacarbazine, CCNU, BCNU, cis-diamminedichloroplatimun, mitoxantrone, CBDCA, levamisole, hexamethylmelamine, all-trans retinoic acid, gliadel and porfimer sodium).

Further examples of chemotherapeutic agents include ixabepilone (BMS-247550, disclosed in Lee et al., Clin. Cancer Res. 7(5):1429-37 (2001)) and INC280 (2-fluoro-N-methyl-4-(7-(quinolin-6-ylmethyl)imidazo[1,2-b][1,2,4]triazin-2-yl)benzamide).

Antiproliferative agents are compounds that decrease the proliferation of cells. Antiproliferative agents include alkylating agents, antimetabolites, enzymes, biological response modifiers, miscellaneous agents, hormones and antagonists, androgen inhibitors (e.g., flutamide and leuprolide acetate), antiestrogens (e.g., tamoxifen citrate and analogs thereof, toremifene, droloxifene and roloxifene), Additional examples of specific antiproliferative agents include, but are not limited to levamisole, gallium nitrate, granisetron, sargramostim strontium-89 chloride, filgrastim, pilocarpine, dexrazoxane, and ondansetron.

The inhibitors of the invention can be administered alone or in combination with other anti-tumor agents, including cytotoxic/antineoplastic agents and anti-angiogenic agents. Cytotoxic/anti-neoplastic agents are defined as agents which attack and kill cancer cells. Some cytotoxic/anti-neoplastic agents are alkylating agents, which alkylate the genetic material in tumor cells, e.g., cis-platin, cyclophosphamide, nitrogen mustard, trimethylene thiophosphoramide, carmustine, busulfan, chlorambucil, belustine, uracil mustard, chlomaphazin, and dacabazine. Other cytotoxic/anti-neoplastic agents are antimetabolites for tumor cells, e.g., cytosine arabinoside, fluorouracil, methotrexate, mercaptopuirine, azathioprime, and procarbazine. Other cytotoxic/anti-neoplastic agents are antibiotics, e.g., doxorubicin, bleomycin, dactinomycin, daunorubicin, mithramycin, mitomycin, mytomycin C, and daunomycin. There are numerous liposomal formulations commercially available for these compounds. Still other cytotoxic/anti-neoplastic agents are mitotic inhibitors (vinca alkaloids). These include vincristine, vinblastine and etoposide. Miscellaneous cytotoxic/anti-neoplastic agents include taxol and its derivatives, L-asparaginase, anti-tumor antibodies, dacarbazine, azacytidine, amsacrine, melphalan, VM-26, ifosfamide, mitoxantrone, and vindesine.

Anti-angiogenic agents are well known to those of skill in the art. Suitable anti-angiogenic agents for use in the methods and compositions of the present disclosure include anti-VEGF antibodies, including humanized and chimeric antibodies, anti-VEGF aptamers and antisense oligonucleotides. Other known inhibitors of angiogenesis include angiostatin, endostatin, interferons, interleukin 1 (including alpha and beta) interleukin 12, retinoic acid, and tissue inhibitors of metalloproteinase-1 and -2 (TIMP-1 and -2). Small molecules, including topoisomerases such as razoxane, a topoisomerase II inhibitor with anti-angiogenic activity, can also be used.

The disclosed compounds can be used in combination with immunotherapy, including but not limited to as interleukin-2 (IL-2), interferon alpha (IFNα), AGS-003 dendritic cell-based vaccine (Escudier, Ann Oncol. 23(suppl 8):viii35-viii40 (2012)), IMA901 containing nine HLA-class I- and one HLAclass II-binding tumour-associated peptides (Escudier, Ann Oncol. 23(suppl 8):viii35-viii40 (2012)), MVA5T4 (Escudier, Ann Oncol. 23(suppl 8):viii35-viii40 (2012)), autologous tumour cell lysate vaccination therapy (Escudier, Ann Oncol. 23(suppl 8):viii35-viii40 (2012)), anti-CTLA-4 antibody ipilimumab (Escudier, Ann Oncol. 23(suppl 8):viii35-viii40 (2012)), anti-PD1 antibody BMS-936558 (Escudier, Ann Oncol. 23(suppl 8):viii35-viii40 (2012)), soluble LAG-3 fusion protein IMP321 (Escudier, Ann Oncol. 23(suppl 8):viii35-viii40 (2012)), and denileukin diftitox (DD) (Escudier, Ann Oncol. 23(suppl 8):viii35-viii40 (2012)).

Other anti-cancer agents that can be used in combination with the disclosed compounds include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RH retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. In one embodiment, the anti-cancer drug is 5-fluorouracil, taxol, or leucovorin.

In certain embodiments, the therapeutic agent is selected from the group consisting of Sorafenib, Sunitinib, Temsirolimus, Everolimus, Bevacizumab, Pazopanib, Axitinib, and Apatinib.

In certain embodiments, the compositions containing the inhibitors described herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts may be determined by methods including, but not limited to, a dose escalation clinical trial.

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. When used in a patient, effective amounts for this use will depend on the severity and course of the disease, disorder or condition, previous therapy, the patient's health status and response to the drugs, and the judgment of the treating physician. In one aspect, prophylactic treatments include administering to a mammal, who previously experienced at least one symptom of the disease being treated and is currently in remission, a pharmaceutical composition comprising the inhibitors described herein.

In certain embodiments wherein the patient's condition does not improve, upon the doctor's discretion the administration of the compounds are administered chronically, that is, for an extended period of time, including throughout the duration of the patient's life in order to ameliorate or otherwise control or limit the symptoms of the patient's disease or condition.

In certain embodiments wherein a patient's status does improve, the dose of drug being administered is temporarily reduced or temporarily suspended for a certain length of time (i.e., a “drug holiday”). In specific embodiments, the length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

Once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the patient requires intermittent treatment on a long-term basis upon any recurrence of symptoms.

In one embodiment, the methods of this invention are useful to a patient who is a human. In one embodiment, the patient is male. In another embodiment, the patient is female. In some embodiments, while the methods as described herein may be useful for treating either males or females, females may respond more advantageously to administration of certain compounds, for certain methods. In other embodiments, while the methods as described herein may be useful for treating either males or females, males may respond more advantageously to administration of certain compounds, for certain methods.

The amount of a given agent that corresponds to such an amount varies depending upon factors such as the particular compound, disease condition and its severity, the identity (e.g., weight, sex) of the subject or host in need of treatment, but nevertheless is determined according to the particular circumstances surrounding the case, including, e.g., the specific agent being administered, the route of administration, the condition being treated, and the subject or host being treated.

In general, however, doses employed for adult human treatment are typically in the range of 0.01 mg-5000 mg per day. In one aspect, doses employed for adult human treatment are from about 1 mg to about 1000 mg per day. In one embodiment, the desired dose is conveniently presented in a single dose or in divided doses administered simultaneously or at appropriate intervals, for example as two, three, four or more sub-doses per day.

In one embodiment, the compositions of the present invention are administered at a dose of between about 0.01 mg per kg of body weight per day (mg/kg/day) to about 100 mg/kg/day, preferably 0.01 mg per kg of body weight per day (mg/kg/day) to 10 mg/kg/day, and most preferably 0.1 to 5.0 mg/kg/day.

In one embodiment, the daily dosages appropriate for the compositions described herein are from about 0.01 to about 50 mg/kg per body weight. In some embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

The compositions of the present invention may be administered orally, topically, transdermally, parenterally, subcutaneously, intravenously, intramuscularly, intraperitoneally, by intranasal instillation, by intracavitary or intravesical instillation, intraocularly, intraarterially, intralesionally, or by application to mucous membranes. In one embodiment, the compositions of the present invention are administered before partial or radical nephrectomy. In another embodiment, the compositions of the present invention are administered after partial or radical nephrectomy.

Toxicity and therapeutic efficacy of such therapeutic regimens are determined by standard pharmaceutical procedures in cell cultures or experimental animals, including, but not limited to, the determination of the LD₅₀ and the ED₅₀. The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the compounds described herein lies within a range of circulating concentrations that include the ED₅₀ with minimal toxicity. In certain embodiments, the daily dosage range and/or the unit dosage amount varies within this range depending upon the dosage form employed and the route of administration utilized.

### Combinations

In one embodiment, the composition of the present invention comprises a combination of an inhibitor of ERβ, an inhibitor of TGFβ-1, an inhibitor of SMAD, or an inhibitor of HIF as described herein.

For example, in one embodiment the composition comprises a combination comprising at least two of an ERβ inhibitor, a TGFβ-1 inhibitor, a SMAD inhibitor, and a HIF inhibitor. In one embodiment, the composition comprises at least three of an ERβ inhibitor, a TGFβ-1 inhibitor, a SMAD inhibitor, and a HIF inhibitor. In one embodiment, the present invention provides a composition, wherein the composition comprises a combination of an ERβ inhibitor, a TGFβ-1 inhibitor, a SMAD inhibitor, and a HIF inhibitor.

In certain embodiments, a composition comprising a combination of inhibitors described herein has an additive effect, wherein the overall effect of the combination is approximately equal to the sum of the effects of each individual inhibitor. In other embodiments, a composition comprising a combination of inhibitors described herein has a synergistic effect, wherein the overall effect of the combination is greater than the sum of the effects of each individual inhibitor.

A composition comprising a combination of inhibitors comprises individual inhibitors in any suitable ratio. For example, in one embodiment, the composition comprises a 1:1 ratio of two individual inhibitors. In one embodiment, the composition comprises a 1:1:1 ratio of three individual inhibitors. However, the combination is not limited to any particular ratio. Rather any ratio that is shown to be effective is encompassed.

### Kits

The invention also includes a kit comprising compounds useful within the methods of the invention and an instructional material that describes, for instance, the method of administering the inhibitors as described elsewhere herein. Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1: ERβ Promotes Renal Cell Carcinoma Progression Via Alteration of TGFβ-1 and EMT Pathway

Renal cell carcinoma (RCC) is the third leading cause of death among urological tumors. Approximately 20-30% of RCC patients present with metastatic disease at the time of diagnosis. Although RCC isn't generally considered as a sex hormones-dependent tumor, it has been reported that incidence of RCC is difference between males and females. Thus, dissecting the roles of estrogen receptor in RCC development is of great interest. Immuno-histochemical staining and RNA expression data showed that ERα is negative and ERβ is positive in RCC tumors. ERβ expression increases as the tumor stages/grades increase. The following study shows ERβ plays a promoting role when tumors develop to higher stages/grades. The data shows that proliferation and migration of the high endogenous ERβ expressing RCC cell lines were significantly increased after estrogen treatment. Using ERβ-shRNA knockdown or cDNA overexpression strategies, the data shows that higher ERβ expression promotes proliferation, migration, and invasion in 786-O and A498 cells. Results from in vivo mouse RCC model show ERβ promotes RCC growth and metastasis, but anti-estrogens can inhibit RCC progression. Further mechanism studies revealed that ERβ could promote RCC progression via increasing the TGFβ-1/SMAD3 signal pathways and the Epithelial-Mesenchymal Transition (EMT). Interruption of TGFβ/SMAD3 via TGFβR-1 inhibitor can lead to suppression of RCC migration. Together, the data shows that ERβ promotes RCC growth and invasion. Targeting ERβ or its downstream TGFβ-1/SMAD3/EMT pathways may be applied as an alternative therapy strategy for metastatic RCC.

The materials and methods employed in these experiments are now described.

Tissue Samples

80 paraffin embedded RCC specimens from 52 male and 28 female patients, 30 adjacent normal kidney tissues, and 6 metastatic specimens from 4 male and 2 female patients between January 2002 and March 2012 were retrieved from the files of the Department of Urology, the First Affiliated Hospital of Medical College of Xi'an Jiaotong University for analysis. All patients provided written informed consent for use of their tissue specimens. The mean age at operation was 63 years (range 33 to 82). All specimens were re-evaluated with respect to pT stage, Fuhrman's grade and histological subtypes by two pathologists. pT stages were adjusted according to the 2009 edition of the TNM system and nuclear grading was performed according to WHO guidelines. Stage pT1, pT2 and pT3 disease was present in 49 (61.3%), 24 (30%) and 7 (8.7%) tumors, respectively. Tumor grade was GI, GII and GIII in 33 (41.3%), 29 (36.3%) and 18 (22.4%) cases, respectively.

A total of 119 cases of RNA from different grade RCC samples tissues were obtained postoperatively from the Department of Urology, Chinese People's Liberation Army General Hospital. The tumors areas were identified by two separate senior pathologists and grade based on the same guidelines

Analysis of the Correlation of ERβ Expression Level and RCC Overall Survival Rate

The TCGA database from Oncomine was analyzed. There are 88 RCC cases with different grades and tumor stages. After 5 years of follow-up, there are 73 cases alive and 15 cases dead.

Cell Culture and Stable Cell Lines

The human RCC cell lines 786-O (ERβ positive), and A498 (ERβ negative) were purchased from ATCC, and the normal human kidney proximal tubular cell line HKC-2/HKC-8 (ERβ positive) were kindly provided by Dr. Syed Khundmiri from University of Louisville (Louisville, Ky.), and maintained in Dulbucco's Minimum Essential Medium (DMEM) (Invitrogen, Carlsbad, Calif.) with 10% fetal bovine serum (FBS). The ERβ knockdown of 786-O and ERβ overexpressing A498 cells were transfected by lentiviral transduction of siRNA ERβ or ERβ cDNA, respectively.

Lentiviral Vector Construction and Virus Production

lentiviral (PLKO.1-puro-shERβ) was constructed with target sequence 5′-GCGAGTAACAAGGGCATGGAA-3′ (SEQ ID NO: 1) or 5′-CTTCAAGGTTTCGAGAGTTAA-3′ (SEQ ID NO: 2). Lentiviral particles were generated by calcium phosphate transfection of lentiviral expressing plasmid, packaging plasmid psPAX2, and envelope plasmid pMD2.G into HEK 293 cells and the lentiviral particles were collected to infect target cells according to Add gene's pLKO.1 protocol.

Cell Proliferation Assay

RCC cells were seeded in 24-well plates (5000 cells/200 μl medium per well) and cultured for 0, 2, 4 and 6 days. Cells were harvested and cell number was calculated using MTT agent.

Colony Formation Assay

Cells were digested and 1×103 cells were seeded into 6 cm plates and incubated for 10 days. The supernatants were discarded, and cells were rinsed in PBS twice and fixed in methanol for 10 min. The cells were appropriately stained with Giemsa's solution (AppliChem, MO) and air dry at room temperature. The experiments were triplicated and the numbers of colonies were counted.

Cell Migration and Invasion Assay

The migration or invasion capability of RCC cells was determined using the transwell assay. In brief, 1×105 (invasion)/2-5×104 (migration) cells were harvested and seeded with serum-free media into the upper chamber with or without coated Matrigel with (BD Corning). The migrated or invaded cells were fixed by 4% paraformaldehyde and stained with 1% toluidine blue. Cell numbers were counted in five randomly chosen microscopic fields (100×) per membrane. When necessary, cells were treated with 10 μM of TGFβR-1 inhibitor (SB525334 Selleckchem) for 48 hr.

RNA Extraction and Quantitative Real-Time PCR Analysis

Total RNA was isolated using Trizol reagent (Invitrogen) according to the manufacturer's instructions. Two μg of total RNA was subjected to reverse transcription using iScript™ Reverse Transcription Supermix (Bio-Rad, Hercules, Calif.). RT-PCR has been described previously (Chen et al., Endocrinology, 2009, 150:251-9). Quantitative real-time PCR (qRT-PCR) was conducted using a Bio-Rad SYBR CFX 96 system.

RT-PCR Assays

The miRNA RT-PCR assays were performed using the TaqMan miRNA Reverse Transcription Kit and the 7500 Fast Real-Time PCR System (Applied Biosystems NY) for quantitative miRNA detection, and each miRNA TaqMan PCR probe was purchased from Applied Biosystems. The real-time RT-PCR assays were performed using the 7500 Fast Real-Time PCR System for quantitative mRNA detection and with iTaq Fast SYBR Green Supermix (Bio-Rad).

Western Blot (WB) Analysis

The expressions of ERs were determined by the Western blot according to the previous study (Hsu et al., Carcinogenesis, 2014, 35:651-61). Rabbit anti-ERβ polyclonal antibody (GTX 110607 GeneTex Corporation), mouse anti-GAPDH monoclonal antibody (Santa Cruz Biotechnology), rabbit anti-TGFB1 polyclonal antibody (Abcam Biotechnology), and mouse anti-SMAD3 monoclonal antibody (Abcam Biotechnology) were all used and all at 1:1,000 dilution in blocking buffer. The immunopositive bands were visualized with an ECL chemiluminescent detection system (Thermo Scientific, Rockford, Ill.), and the images were transferred to the Bio-rad imaging system. The Western analysis was scored positive if the band of interest was present at the expected molecular weight appropriate for each marker protein. All analyses were done in duplicate.

Subcutaneous and Renal Capsule Implantation

The Fe-NTA transformed human renal carcinoma cell lines 7860.sh-ERβ/7860.sh-Luc and A498 hERβ/Vec were directly injected into the renal capsule of nude mice. After anesthesia, the back skin was lifted with a pair of blunt forceps and cut along the midline with a pair of scissors creating an incision approximately an inch in length; the dermis was separated from the underlying body wall on both sides of the incision for bilateral grafting or on one side only (blunt dissection). The animal was placed on its side, and the location of the kidney identified by viewing it through the body wall. A small incision (3-4 cm) was made in the body wall just slightly longer than the long axis of the kidney. The exteriorized kidney was allowed to rest on the body wall. We used a syringe to inject the cells at the cell numbers of 1×10⁶, both with matrigel (1:1) under the renal capsule.

Immunohistochemistry (IHC) and Immunofluorescent (IF) Staining

IHC and IF staining was carried out as described previously (Chen et al., Endocrinology, 2009, 150:251-9; Chen et al., Carcinogenesis, 2009, 30:841-50). Tumor sections were placed on slides and were incubated with the following primary antibodies: Mouse polyclonal antibody against ERα (Leica Microsystes 6F11 1:100), Mouse monoclonal to ERβ (Abcam 14C8 1:50), rabbit anti-TGFβ1 polyclonal antibody (Abcam ab27969 1:100), N-Cadherin Rabbit mAB (CellSignaling D4R1H 1:200) were used and anti-firefly (Santa Cruz Biotechnology) in 3% bovine serum albumin in phosphate-buffered saline overnight at 4° C., followed by respective secondary antibodies. The stained slides were mounted and visualized by a bright-field microscope or fluorescent microscope.

The specificity of IHC staining for human RCC patient samples studied protein expressions. The expression of ERβ was investigated in semi-quantitative fashion independently by two pathologists. The intensity of staining was graded from 0 to 3+ as follows: 0, no staining; 1+, weak, light beige staining; 2+, easily visible staining affecting most cells with a tan coloration; 3+, strong, diffuse brown staining of the cells (Jinushi et al., Cancer Res, 2008, 68:8889-98).

Statistical Analysis

Quantitative data are presented as mean±SE, statistical significance among control group and treated groups were accomplished by ANOVA. p<0.05 was regarded as the threshold value for statistical significance.

The results of the experiments are now described.

ERα Expression is Negative, but ERβ Expression Increases with Stage and Grade Rise in RCC Patients.

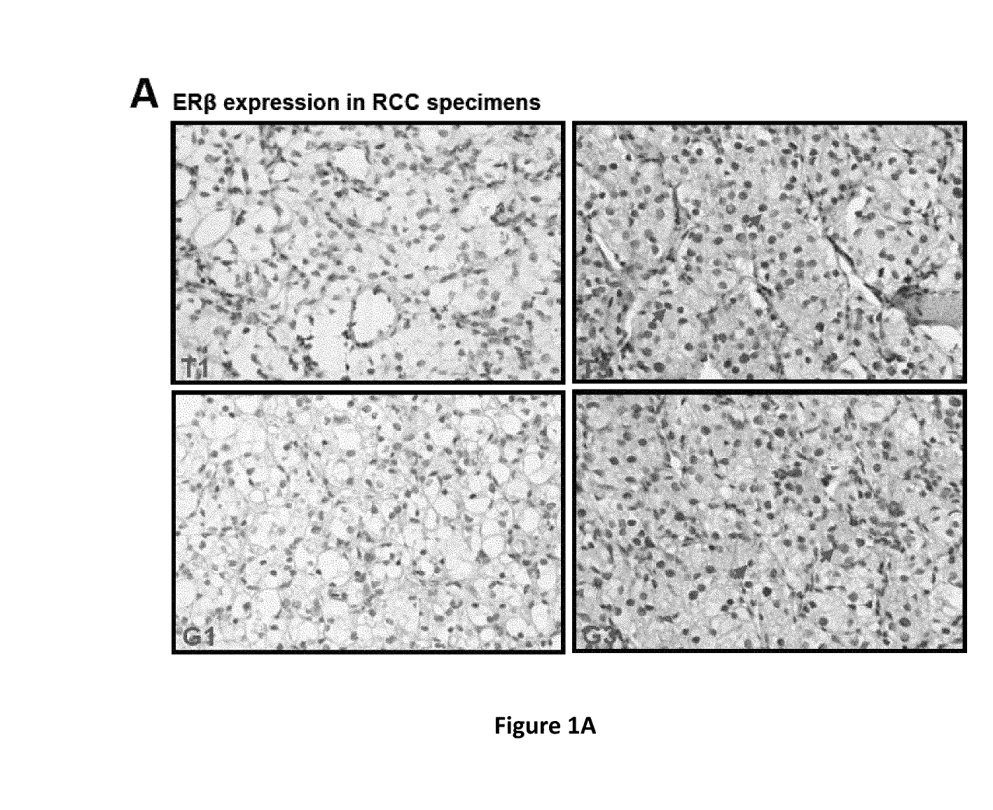

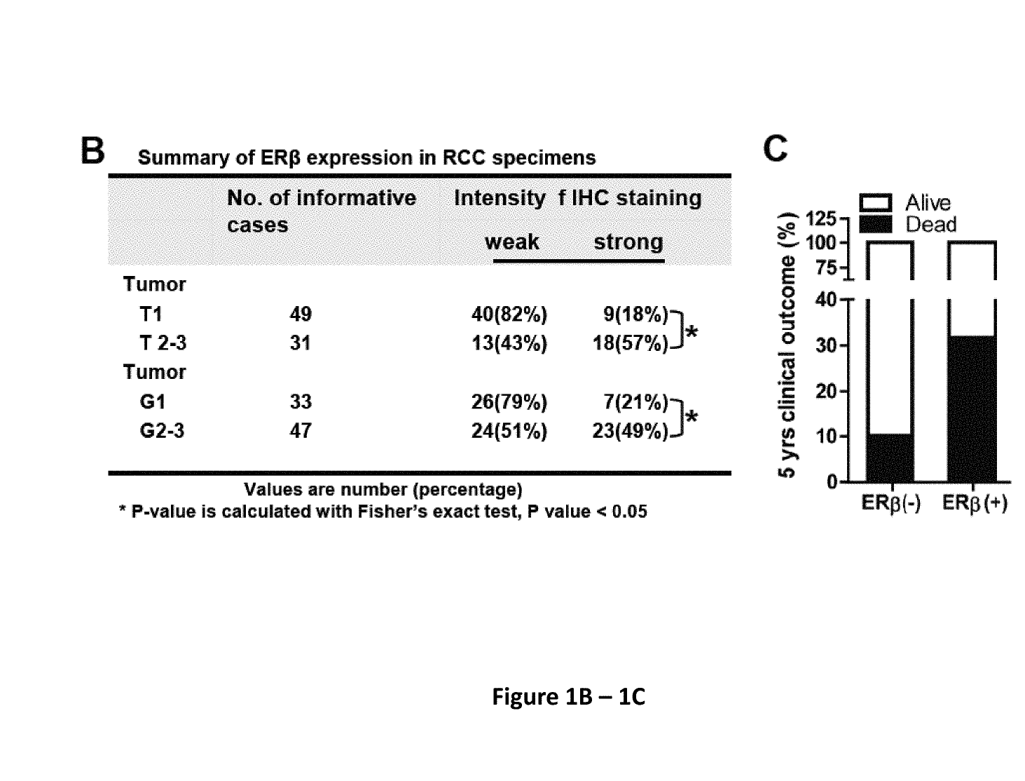

To understand the ERα and ERβ expression in RCC, IHC was performed with ERα and ERβ (Lit et al., Anticancer Res, 2013, 33:53-63; Lim et al., Breast Cancer Res, 2011, 13:R32) antibodies in 80 formalin fixed RCC specimens with either high or low stage/grade of RCC tissues from patients with median age of 63. Interestingly, it was found that ERβ was detectable, yet ERα expression was undetectable in these RCC samples. Comparison of the ERβ expression in different stages of RCC from 49 pT1 stage and 31 pT 2-3 stage of RCC tumors showed that 18% (9 of 49) pT1 stage RCC tumors had strong ERβ expression compared with 57% (18 of 31) pT2-3 stage RCC tumors (p=0.0005) (FIG. 1A). Similar conclusions also occurred in different pathological grades showing 21% (7 of 33) Fuhrman's grade 1 (G1) RCC tumors had stronger ERβ expression compared with 49% (23 of 47) grade G2-3 RCC tumors (p=0.0184, FIG. 1B). In addition to detecting the ERβ protein by IHC staining, RNA was extracted from 119 different clinical specimens with either high or low grade of RCC. Results showed that ERβ mRNA expression increased with grade increases. Analysis of TCGA Renal Dataset via the Oncomine website shows that there are a total of 88 RCC cases. Among those 88 cases, 28 RCC cases have positive ERβ expression, and 9 of 28 (32.1%) were dead in five years. The rest of the 60 cases have no (or little) ERβ expression, and 6 of these 60 (10.0%) were dead by the five years follow-up. Indeed, ERβ positive RCC patients have higher death rate (32.1% vs 10.0%, FIG. 1C).

Together, consistent results from FIG. 1 suggest that there is a higher nuclear ERβ staining (IHC) and mRNA levels (Q-PCR) in G2-3 stage of RCC compared to G1 stage of RCC. RCC patients with ERβ(+) expression have a worse clinical prognosis with a reduced 5 years overall survival.

### ERβ Expression and Function in RCC Cells.

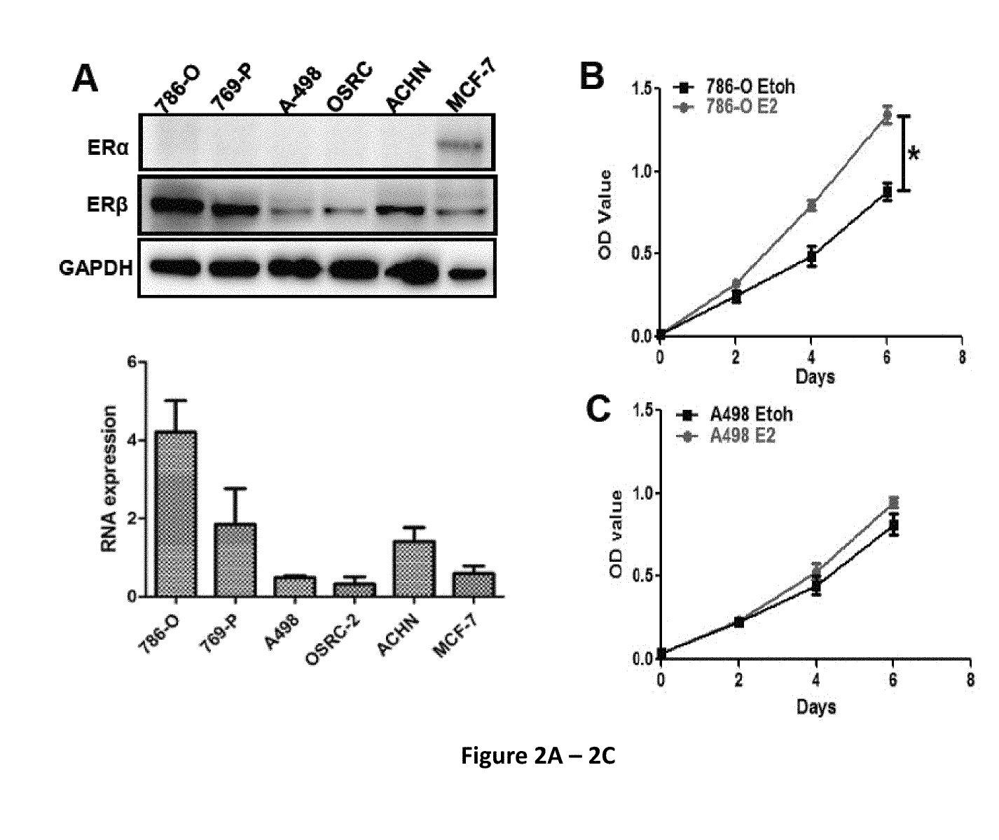

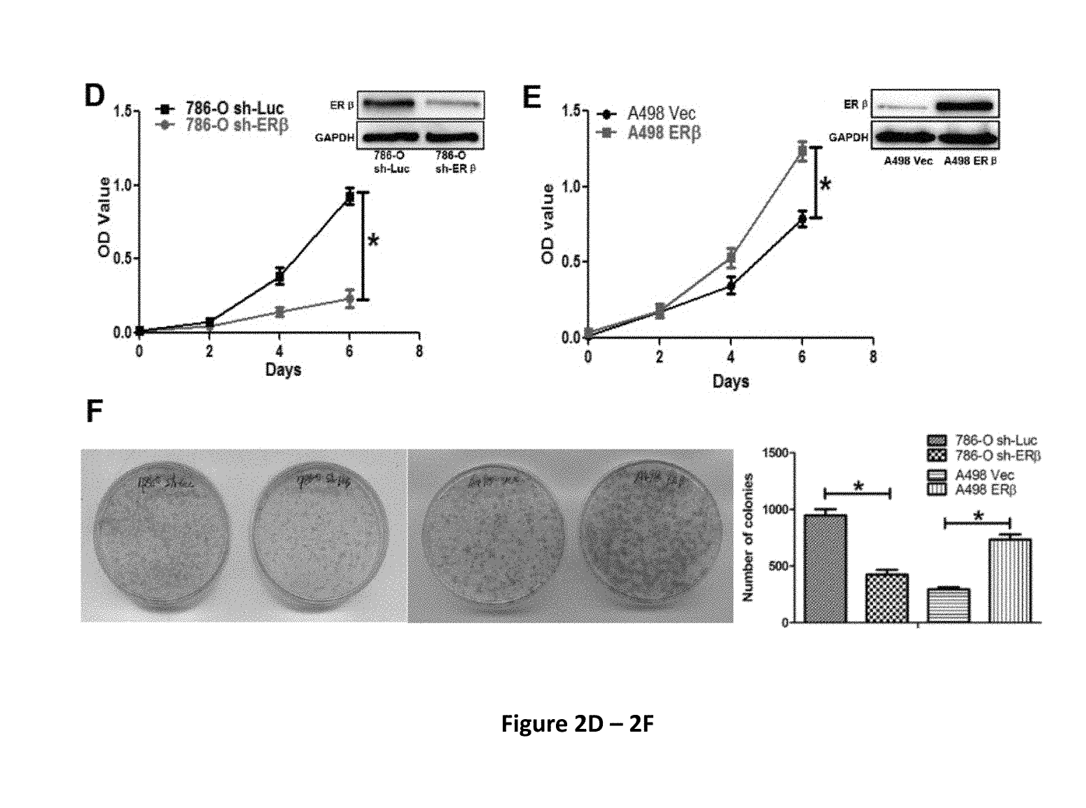

ERβ expression was validated in various RCC cell lines and it was found that ERβ was strongly expressed in 786-O and 769-P cells and weakly expressed in A498 and OSRC-2 cells, MCF-7 cells as positive control for ERα and ERβ detection (FIG. 2A). Consistent with clinical tissue analysis, ERα is undetectable and ERβ expression was detected in examined 5 different RCC cell lines.

To examine the ERβ roles on RCC cell growth, the lentiviral ERβ cDNA and shRNA were used to change the expression of ERβ in 2 RCC cell lines, A498 (low endogenous ERβ) and 786-O (high endogenous ERβ), respectively. 10 nM 1743 estradiol (E2) was used to activate ERβ and examine the growth changes using MTT assay and colony forming assays. The results showed that E2 significantly enhanced the cell growth in 786-O cells (FIG. 2B). In contrast, little E2 stimulating effect was detected in A498 cells that expressed little ERβ (FIG. 2C). Supportively, knocking-down ERβ by ERβ-siRNA in RCC 786-O cells suppressed cell growth, sh-Luciferase as control (FIG. 2D). When ERβ was over-expressed in A498 cells (by lentiviral ERβ cDNA), the increased ERβ expression could induce A498 cell growth (FIG. 2E). The colony formation assay produced similar results (FIG. 2F). Together, results from FIG. 2 suggest E2/ERβ play positive roles to promote RCC cell growth.

### ERβ Promotes RCC Cell Migration and Invasion

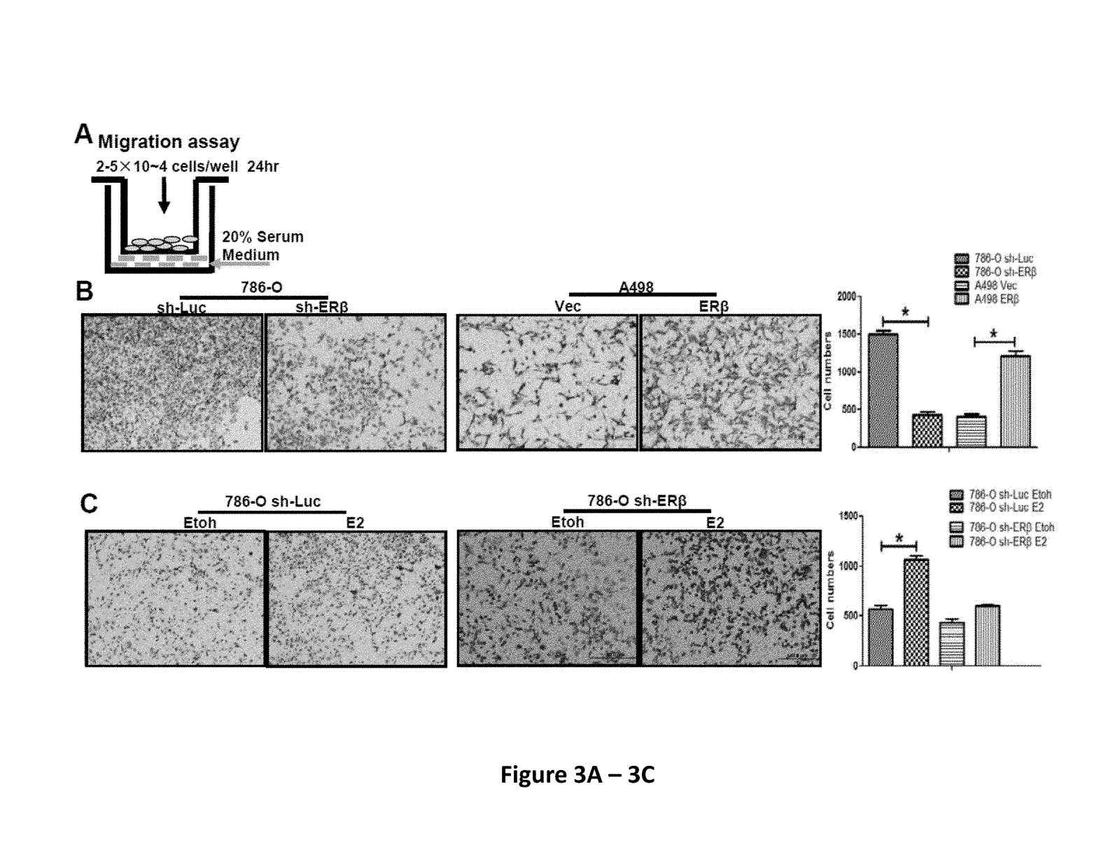

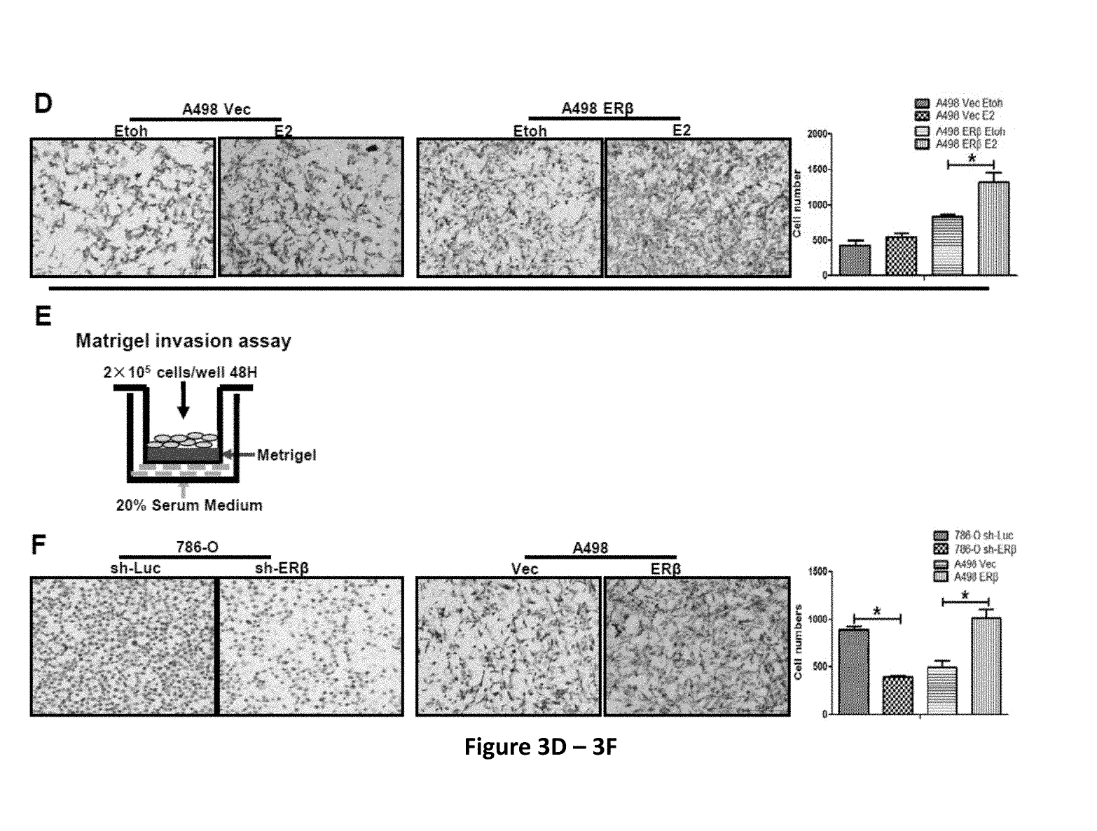

To examine the ERβ effect on RCC metastasis, the transwell system (FIG. 3A) was applied to assay the RCC cell migration and invasion abilities. As shown in FIG. 3B left panel, knockdown of ERβ suppressed the migration of 786-O cells. Furthermore, treatment with 10 nM E2 could enhance 786-O sh-Luc control cell migration (FIG. 3C, Left), and E2 treatment had little effect in the ERβ knockdown 786-O cells (786-O-shERβ) (FIG. 3C, Right), suggesting E2 functions via ERβ to promote RCC migration. Supportively, up-regulation of ERβ in A498 cells could increase cell migration as compared with A498-Vector control cells (FIG. 3B, Right) and 10 nM E2 treatment further stimulated the migration in A498-ERβ cells (FIG. 3D, Right). As illustrated in FIG. 3F, ERβ effects on RCC invasion were further tested by the transwell matrigel assay. The results showed that knockdown of ERβ in 786-O cells (786-O sh-ERβ) suppressed cell invasion (FIG. 3F Left). In contrast, addition of functional ERβ increased invasion in A498 cells (FIG. 3F Right).

Together, results from FIG. 3 showed that E2 could function through activation of ERβ to enhance the RCC cell migration and invasion in 2 pairs of RCC cells with high vs low ERβ expression.

ERβ Alters Cell Morphology with Increased EMT and MiRNA in RCC Cells

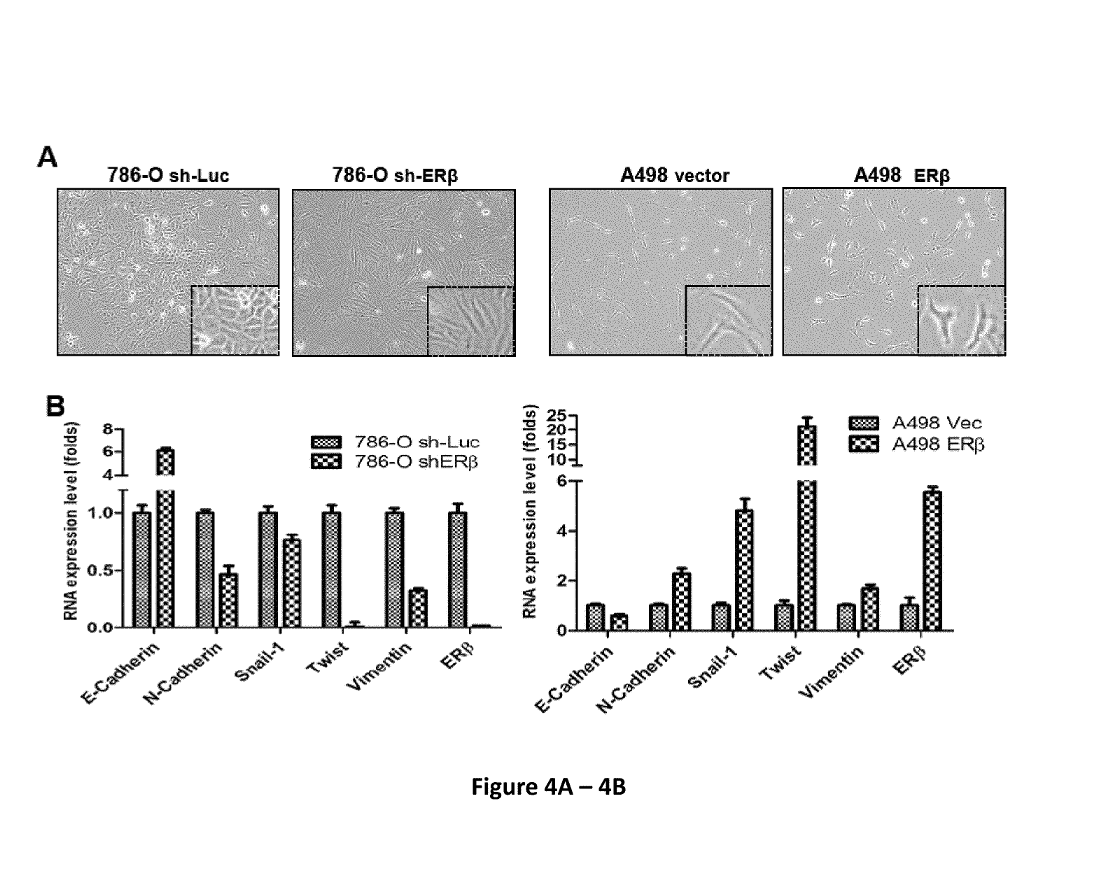

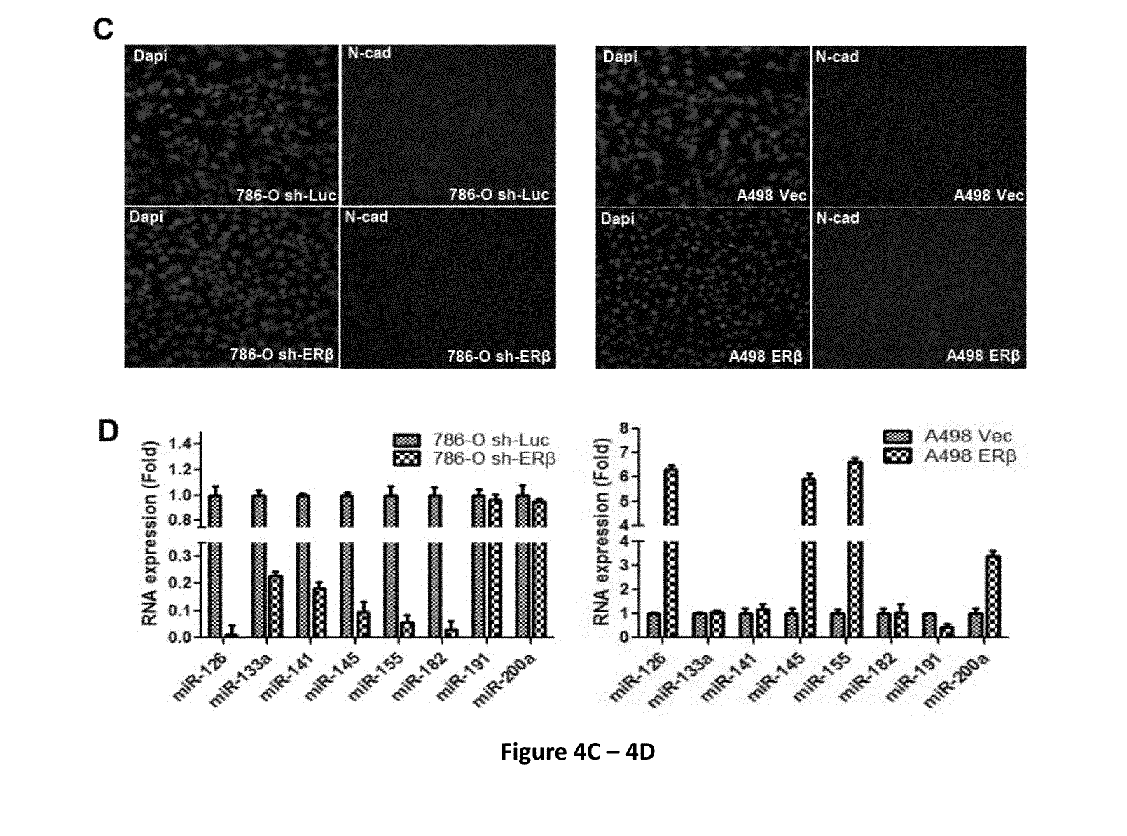

Changes in cell morphology were evident after alteration of ERβ levels within cells, such as becoming slender when ERβ is knocked-down in 786-O cells, or becoming spindle shaped with the ectopic expression of ERβ in A498 cells (FIG. 4A). Importantly, when ERβ is knocked-down in 786-O cells, the morphology changes are accompanied with the EMT marker changes decreased expressions of N-Cadherin, snail, twist and vimentin as well as an increased E-Cadherin (FIG. 4B), suggesting ERβ may be able to function through modulating EMT to alter the RCC cell morphology. As EMT plays key roles in cell invasion, these results also demonstrate that ERβ may function through modulation of EMT to influence RCC cell invasion. In addition, immunofluroscence staining was applied to detect N-Cadherin expression in RCC with changed ERβ expressions. Similar results showed that N-Cadherin expression was increased when the RCC cells have more ERβ, 786-O sh-Luc vs 786-O sh-ERβ and A498 ERβ vs A498 vec (FIG. 4C). Furthermore, a group of metastasis/EMT related miRNAs was examined using Q-PCR and results showed miRNA126, miRNA145 and miRNA155 were up-regulated in RCC cells with higher ERβ (FIG. 4D). Among those miRNAs, miRNA 145 is the downstream effector of TGBβ1 signal. The changes of miRNA145 data support the correlative TGFβ1 pathway changes in RCC cells with higher ERβ signals.

### TGFβ-1/SMAD3 as a Key Player for ERβ-Mediated RCC Cell Migration/Invasion

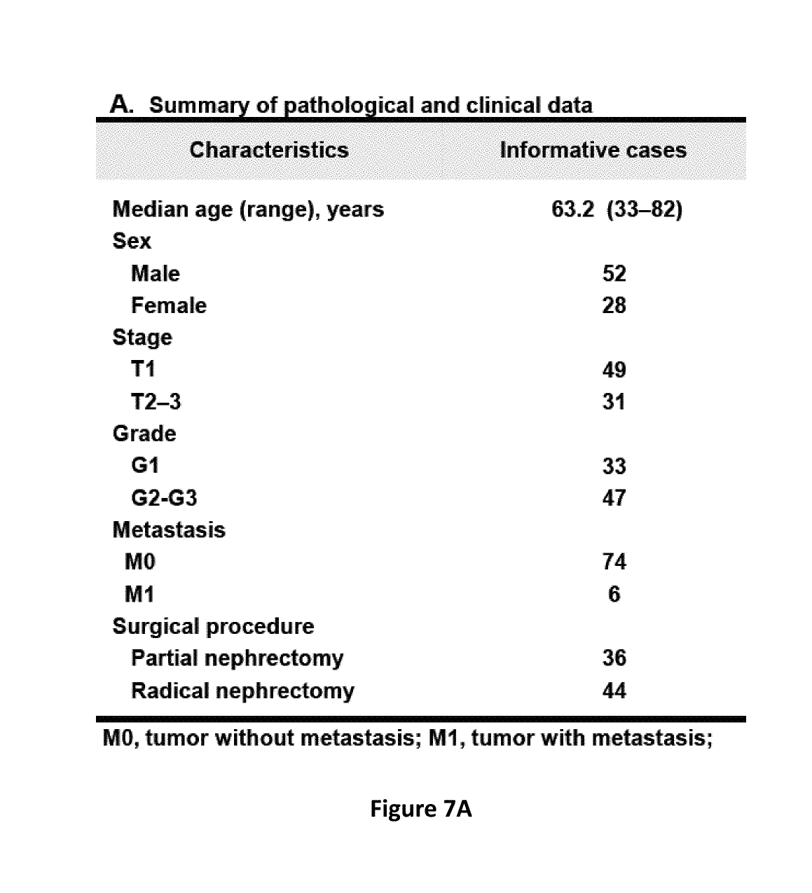

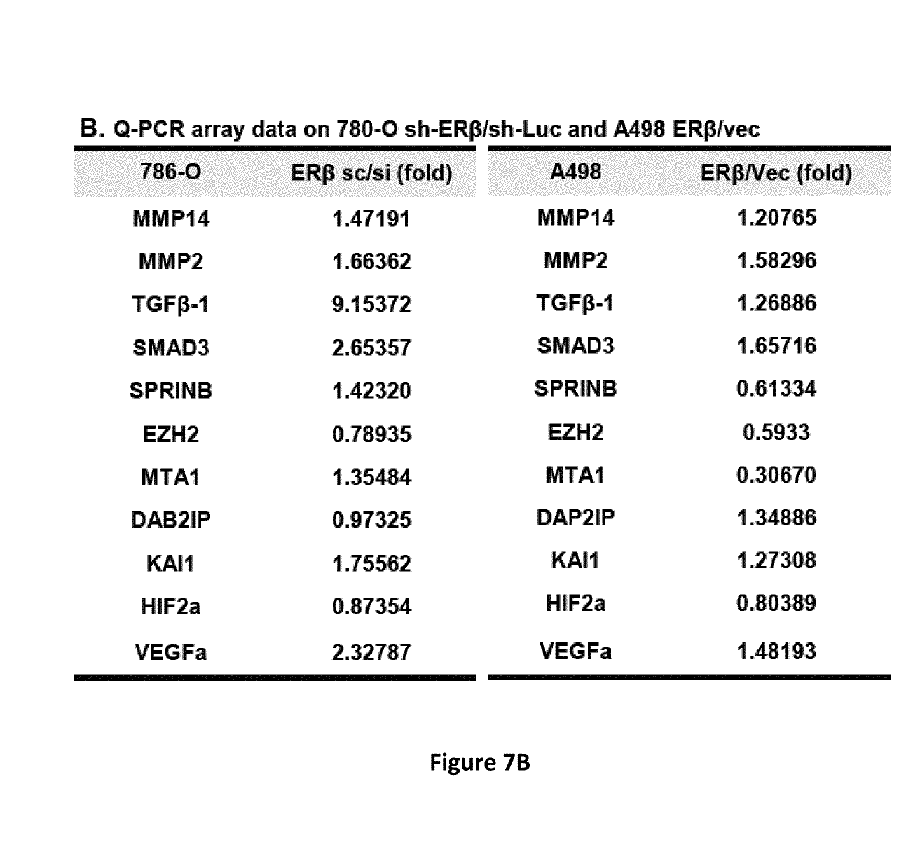

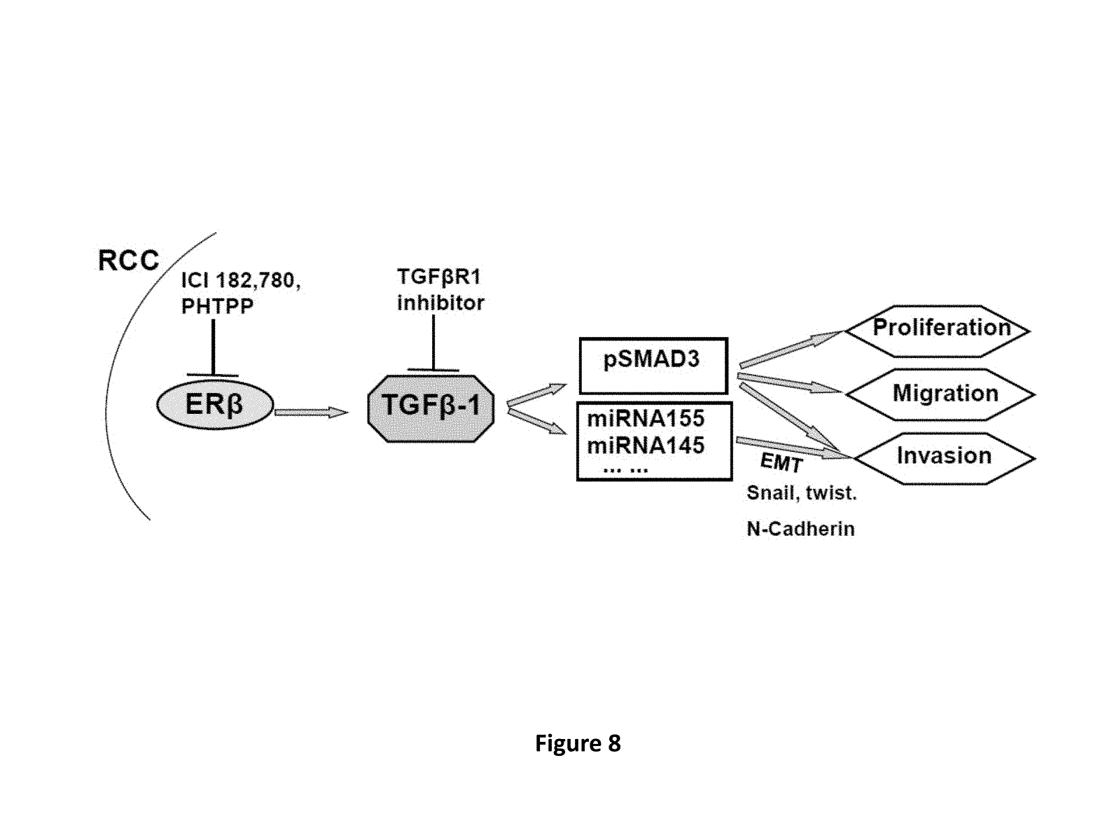

To further dissect the molecular mechanism(s) by which ERβ influences RCC cell migration/invasion, Q-PCR-based focus-array analyses were applied to search for the key metastasis related genes that are responsible for ERβ-enhanced RCC progression. Among many metastasis-related genes (FIG. 7B), consistent and significant changes in TGFβ-1 and SMAD3 were found, whose expression of ERβ were much higher than those in negative or low ERβ RCC cells (p<0.01). Furthermore, early reports have well documented that TGFβ-1 and SMAD3 could play key roles in cancer metastasis. These findings showed a promising connection of the ERβ signals to TGFβ-1 and SMAD3 pathways in RCC.

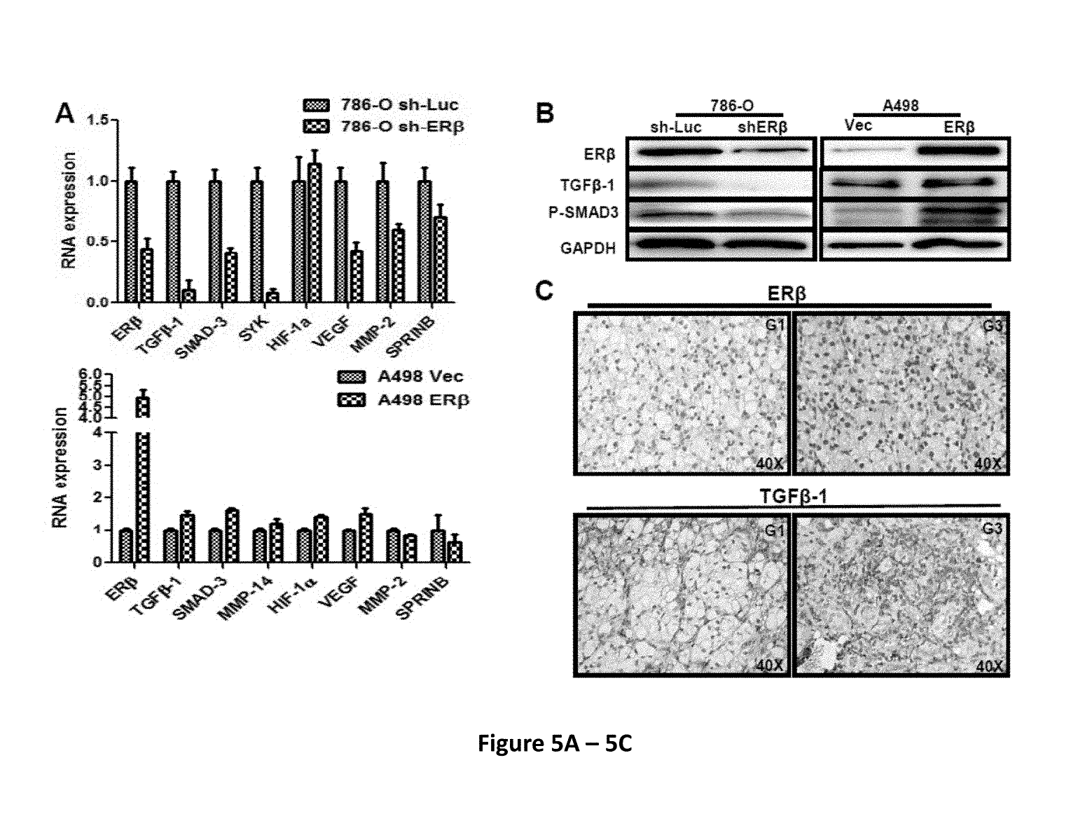

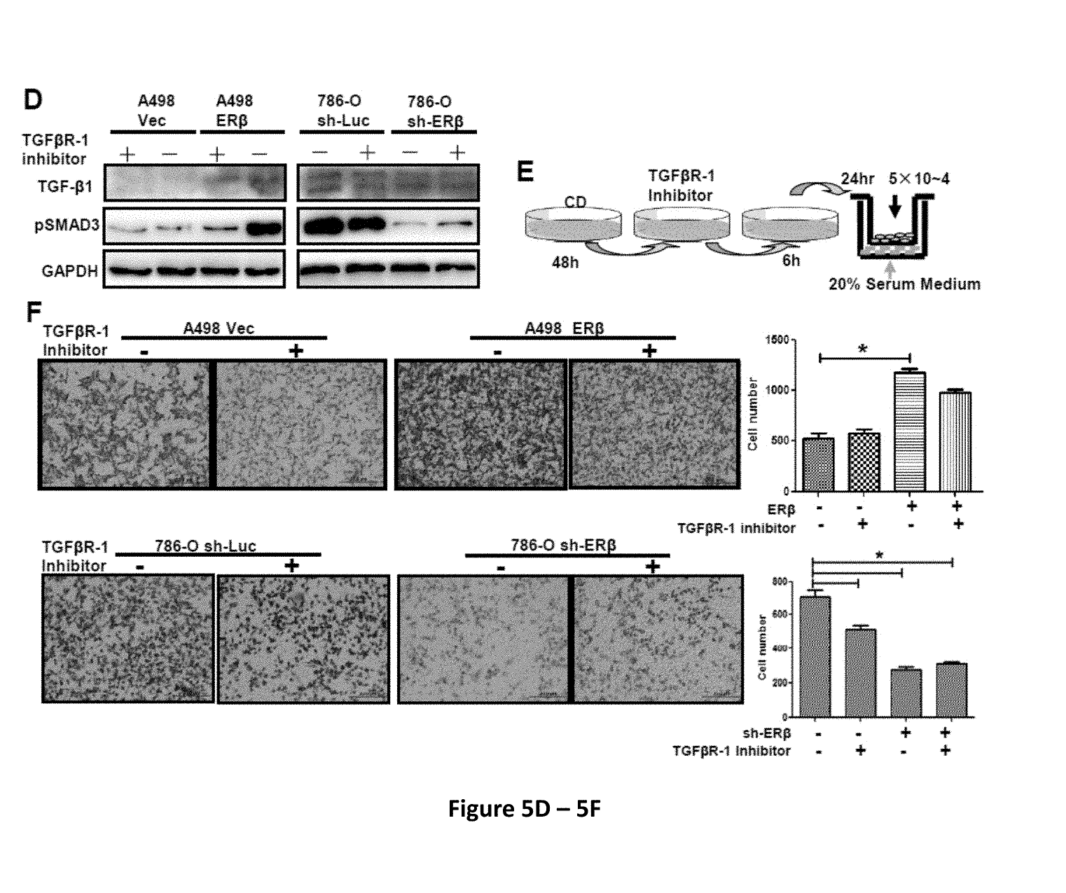

Western blot (WB) and Q-PCR assays were applied to confirm these two genes could be modulated by ERβ at mRNA (FIG. 5A) and protein levels (FIG. 5B) in both 786-O and A498 cells. In addition, this ERβ modulated expression of TGFβ-1 and SMAD3 was investigated in human RCC samples. Clinical tissue IHC staining results showed the expression of ERβ and TGFβ expression) are increased in higher pT stage and Fuhrman's grade in RCC patients (FIG. 5C).

To further validate the ERβ→TGFβ1→pSMAD3 pathway, an interruption approach was applied by using TGFβR-1 inhibitors (10 μM) for the cell invasion assay in both 786-O and A498 cells (FIG. 5D). It was found that treatment of TGFβR-1 inhibitor led to the suppression of ERβ-enhanced invasion in both 786-O and A498 cells (FIGS. 5E and 5F), suggesting that TGFβ-1→SMAD3 pathway plays important roles in ERβ mediated RCC cell invasion. Together, results from FIG. 5 demonstrated that the ERβ could function through its downstream targets, TGFβ-1 and SMAD3, to enhance RCC cell invasion.

### ERβ Promoted RCC Growth and Invasion in In Vivo Mouse Models.

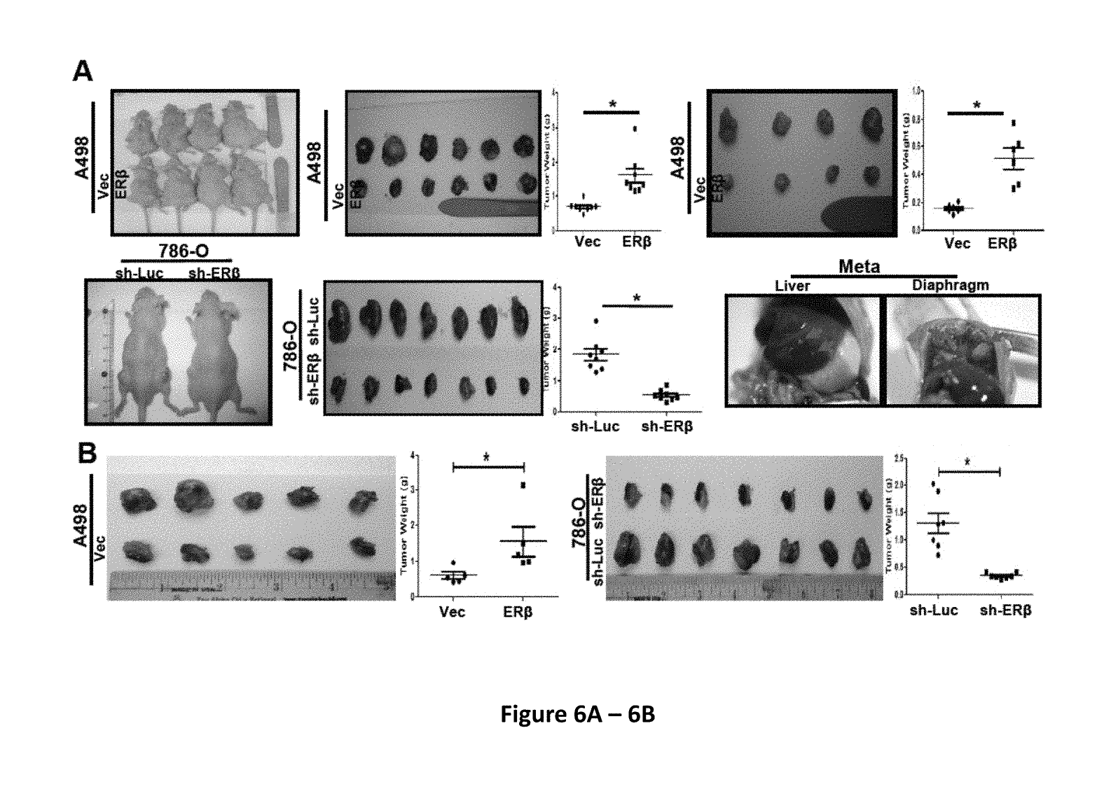

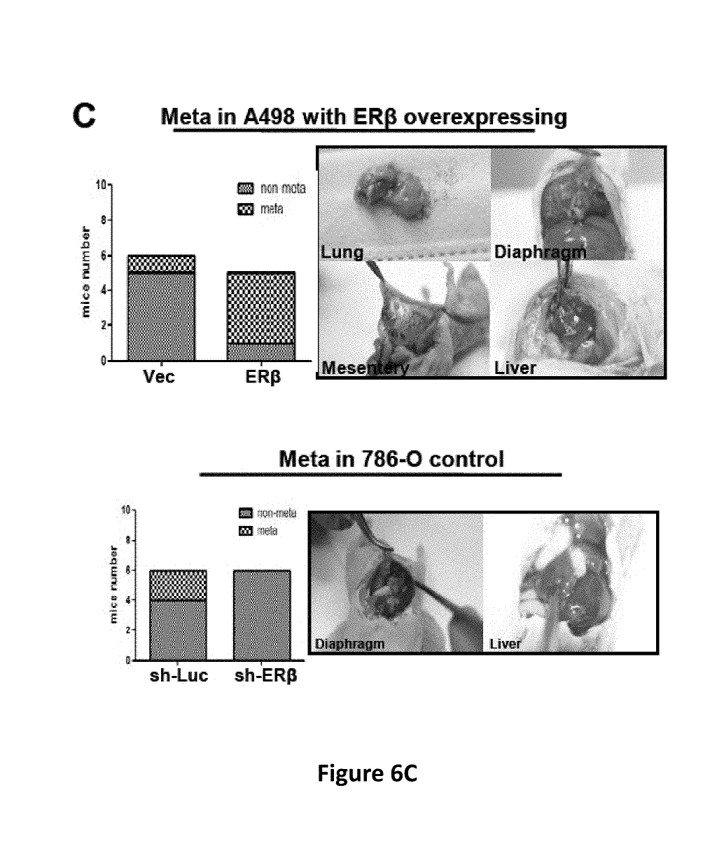

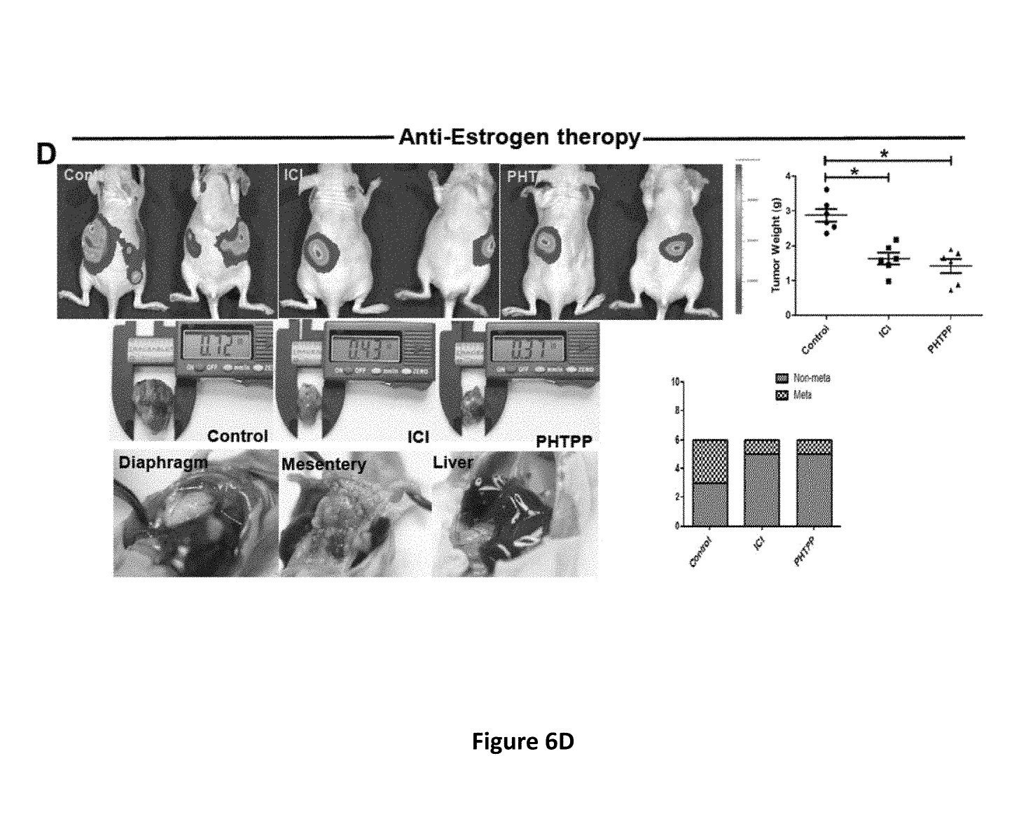

To further confirm the above in vitro cell lines data with in vivo mouse model, human RCC 786-O sh-ERβ/sh-Luc and A498-hERβ/Vec tumor cells were implanted into male and female mice, subcutaneously and orthotopically. The results indicated ERβ could promote the RCC progression in in vivo male RCC model (FIG. 6A), as well as female RCC model (FIG. 6B). Consistent results were obtained from both subcutaneous xenografts and orthopotic xenografts (under the renal capsule).

Interestingly, it was found that RCC cell grafts with higher ERβ develop more distal metastatic tumors both in female than male mice group. In female group, 4 out of 5 mice with orthotopically renal implanted A498 ERβ cells formed diaphragm, liver, lung and mesentery metastasis and 1 of 6 mice has metastasis in A498 vec control group (with lower ERβ). Two out of 6 mice in 786-O sh-Luc formed distal metastasis, and none of 786-O sh-ERβ group (ERβ knockdown) has metastasis. In male group 2 out of 6 mice with orthotopic implantation of A498 cells with over expressed ERβ (A498 ERβ) had metastatic tumors in the diaphragm, liver and hepatic portal lymph node as compared to 0 out of 6 mice with A498 vec control implants (FIG. 6C). Histology analyses further confirmed those tumors are RCC and metastatic tumors.

### ERβ Induced the Expression of TGFβ-1/SMAD3 in the Xenografted RCC Tumors

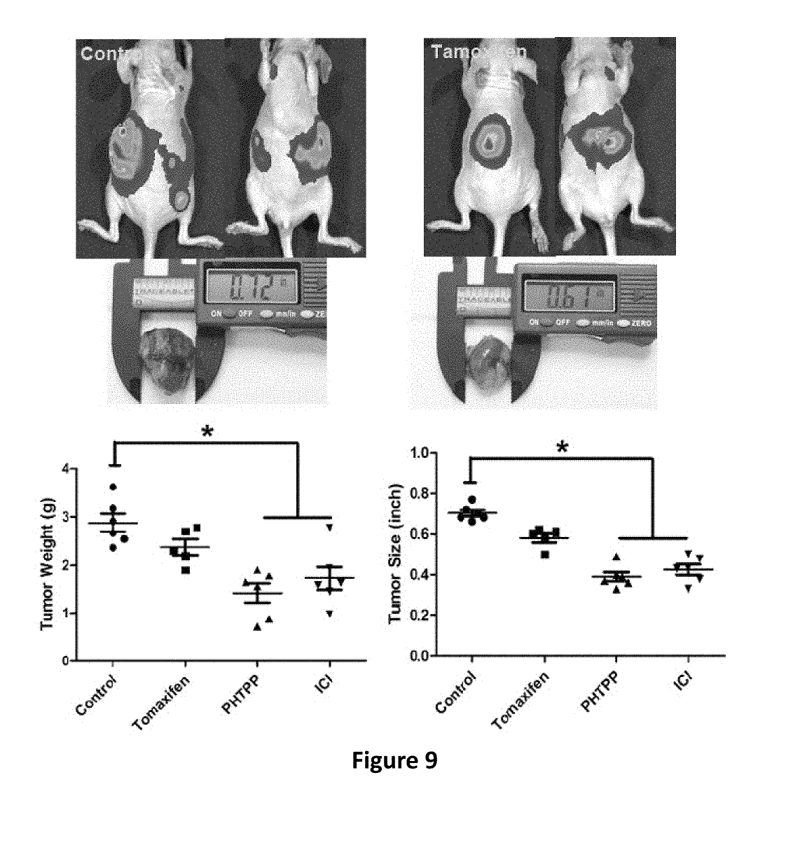

IHC staining of xenografted RCC tumors from nude-mice demonstrated that high expression of ERβ positively correlated with higher expression of TGFβ-1/SMAD3 in the orthotopically xenografted RCC tumors by comparing A498 ERβ vs A498 vec cells and 7860 sh-ERβ vs 7860 sh-Luc cells. Taken together, in vivo data from subcutaneously and orthotopically xenografted RCC mouse models demonstrated that ERβ promotes RCC growth and invasion via modulation of the TGFβ-1→SMAD3 pathway. ERβ antagonists, PHTPP and ICI, inhibit RCC cell growth and cell invasion Knockdown of ERβ reduced RCC cells growth and Invasion. It was then investigated whether PHTPP (ERβ selective antagonist) and ICI 182,780 (ICI, anti-estrogen) have therapeutic effects to reduce RCC tumor growth and metastasis in vivo. Previous reports have used 10 μM PHTPP or ICI to effectively inhibit estrogen induced ERβ transactivation on EREluciferase reporter activity. Also, both anti-estrogens have been used to treat mouse breast cancer models. The same doses of PHTPP and ICI were applied in mice carrying 786-O tumors DMSO (mock control), PHTPP and ICI were administrated via intraperitoneal (ip) injection every other day. IVIS imaging was applied to monitor tumor growth and distal metastasis. After 4 weeks of treatments, mice were sacrificed for tumor analysis. It was found that the PHTPP and ICI treated group had higher body weights than control without any signs of toxicity or adverse treatment side effects. All the mice had developed RCC by the time they were sacrificed. It was found that 3 of the 6 DMSO treated mice developed metastasis while only around 17% of PHTPP or ICI treated mice developed distal metastasis (FIG. 6D). In addition to PHTPP and ICI, tamoxifen was also tested on mouse RCC growth. As tamaxifen is not an effective antagonist for ERβ, results indeed showed the tamoxifen treatment is less effective on inhibiting RCC tumor (FIG. 9). Together, the preclinical RCC tumor model data demonstrated that PHTPP and ICI treatment had reduced cancer progression, growth and invasion.

### ERβ Expression Increased as RCC Progressed to More Advanced Stages and the Specific Nuclear Immune Staining of ERβ in Clinical Specimens.

Estrogen is the main female hormone involved in various cell processes, including growth, differentiation, and reproductive function. It interacts with two main types of ERs, ERα and ERβ. After binding to the receptors, estrogen exerts its genomic or non-genomic functions through various signaling pathways. However, whether ERα or ERβ could affect RCC initiation or progression remains to be further elucidated. Through the human renal tissue IHC staining, it was found that ERα is negative, yet ERβ is positively located in the nuclei of RCC tissues. Interestingly, it was found that ERβ expression increased while RCC progresses to the later stages (T1 vs T2-3) or higher grades (G1 vs G2-3) (FIG. 1B). It is well known that both ERα and ERβ are nuclear proteins and several reports have shown the nuclear staining of ERs. Recently, there was a report showing ERβ expression is reduced higher grade RCC and may play an inhibiting role for RCC development (Yu et al., PloS ONE, 2013; 8:e56667). However, there have been concerns regarding the histology ERβ signal detected by rabbit anti-ERβ (Epitomics) did not show a clear nuclear staining in that report. Also, this rabbit anti-ERβ (Epitomics) is not used by other well established researchers in the nuclear receptor field.

Several available antibodies for ERβ have been tested and the specificity of the antibodies has been proven by using endogenous ERβ with or without shRNA knockdown. In addition, in cells without any endogenous ERs, ERβ was transfected into them to test its specificity. To carefully evaluate the clinical staining data, 2 independent pathologists in URMC and Johns Hopkins have contributed to validate the specificity of ERβ antibodies. The validations have identified an ERβ antibody (Abcam 14C8) that could specifically recognize ERβ with nuclear staining signals. The mouse monoclonal antibody of ERβ that was used has been used in several publications to detect ERβ in human tissues (Rudolph et al., Cancer Res, 2013, 73:3306-15). In addition to IHC staining of clinical specimens, multiple strategies were used and 4 different RCC cell lines were purchased from a reliable source, ATCC, and knockdown or ectopic overexpression of ERβ and in vivo and in vitro strategies all proved that ERβ plays a promoting role in RCC growth and invasion.

### Differential ERβ Roles in Different Cancers and Diseases

Estrogens regulate the growth, development and functioning of normal as well as cancerous breast (Russo et al., Medicina-Buenos Aire, 1997, 57:81-91). ERα and ERβ have diverged early during evolution (Kelley et al., J Mol Evol, 1999, 49:609-14) and differ mostly in the N-terminal A/B domain, and to a lesser extent in the ligand binding domain (E domain). These differences suggest that the two receptors could have distinct actions. To date, there are many studies in humans to indicate that the ERβ expression is decreased between normal and neoplastic breast, as well as colon and ovarian cancer (Campbell-Thompson et al., Cancer Res, 2001, 61:632-40; Iwao et al., Int J Cancer, 2000, 88:733-6), suggesting that ERβ could be an inhibitor of tumor initiation in these cancers.

In urinary tract tumors, intra-prostatic metabolism of testosterone to estrogens has been recently proposed to play a vital role in the regulation of prostate gland growth (Muthusamy et al., P Natl Acad Sci USA, 2011, 108:20090-4). ERβ is detected in the prostate gland (Kuiper et al., P Natl Acad Sci USA, 1996, 93:5925-30), and estrogen signaling driven by ERβ has been shown to negatively regulate prostate gland growth (Carruba et al., J Cell Biochem, 2007, 102:899-911). The selective modulators of ERβ activities are effective agents against human prostate cancer invasion and metastasis. Interestingly, in BCa, reports suggested that ERβ could play positive roles in promoting bladder cancer progression and sh-ERβ or ERβ antagonist could inhibit the BCa growth (Hsu et al., Carcinogenesis, 2014, 35:651-61), and mechanism studies showed ERβ could up-regulate MCM5 to control BCa (Hsu et al., Carcinogenesis, 2014, 35:651-61).

Although RCC isn't generally considered as a sex hormones-dependent tumor, it was reported that males have a slightly higher rate than females. A multiple center clinical trial has used the ER antagonist, tamoxifen, to treat metastatic RCC. Some research centers showed that the tamoxifen treatment can affect RCC patients prognosis (Al-Sarraf et al., Cancer Treat Rep, 1980, 65:447-51), but others showed that the treatment does not influence RCC patients progression (Papac et al., Eur J Cancer, 1993, 29:997-9). Without wishing to be bound by any particular theory, it is believed that the inconclusive clinical outcomes could be due to the fact that some patients were ERβ positive and the others were ERβ negative.

### ERβ Isoforms Expression and Function in RCC Remain to be Determined

There are 5 major isoforms of ERβ, including β1, β2, β3, β4, and β5 (Leung et al., P Natl Acad Sci USA, 2006, 103:13162-7). Sequencing data demonstrated that multiple ERβ isoforms could be a result of alternative splicing of the last coding exon (Moore et al., Biochem Bioph Res Co, 1998, 247:75-8). Previously, published data on human ERβ function signaling was mainly based on the characterization of ERβ-1, the one originally cloned (Mosselman et al., FEBS Lett, 1996, 392:49-53). Limited studies, however, have revealed that ERβ-2 (ERβcx) functions as a dominant negative of ERα (Ogawa et al., Nucleic Acids Res, 1998, 26:3505-12), whereas ERβ-3 expression appears to be restricted to the testis (Moore et al., Biochem Bioph Res Co, 1998, 247:75-8). Additionally, two truncated transcripts, containing only part of the common exon 7 and different exon 8 sequences, have been identified and named ERβ-4 and ERβ-5 (Moore et al., Biochem Bioph Res Co, 1998, 247:75-8). To date, the functional properties of the individual ERβ isoforms remains to be elucidated except for their differential expression in various tissues and cell lines (Moore et al., Biochem Bioph Res Co, 1998, 247:75-8). Without a comprehensive understanding of the functional uniqueness and similarity of these isoforms, the biological significance of ERβ in RCC remains to be explored. In the present study, the focus was on characterizing ERβ-1 function in RCC. The study also encompasses detecting the expression of different ERβ isoforms in RCC specimens and cell lines.

### ERβ Selectively Regulates TGFβ-1 Signals, not EGF, Insulin, or IGF-1.

Some studies showed that ERs could function as transmitters of signals from various growth factors including EGF, insulin and IGF-I (Newton et al., J Mol Endocrinol, 1994, 12:303-12) in ER positive cells transfected with an ERE-containing reporter plasmid. Other than functioning as transmitter, ERα and ERβ could regulate the expression or functions of those signal pathways. Interestingly, it was observed that ERβ could selectively up-regulate TGFβ-1, but not EGF, insulin or IGF-1 expression in RCC cells (FIG. 4).

In the present study, it was found that higher ERβ could up-regulate TGFβ-1, which subsequently influences the function of the downstream gene, SMAD3. Thus, the effect of ERβ on the TGFβ-1 signaling pathway was investigated in the promotion of tumor progression. The results of this study showed that TGFβ-1 stimulation in 786-O RCC cells (with high endogenous ERβ expression) resulted in promoting RCC progression. TGFβ-1 signaling is mediated through two types of transmembrane serine/threonine kinase receptors (Massague et al., Cell, 1996, 85:947-50). Upon binding to TGFβ-1, the type II TGFβ receptor (TbRII) forms a heteromer with the type I TGF-β receptor (TbRI), resulting in the phosphorylation and activation of TbRI (Wrana et al., Nature, 1994, 370:341-6). The activated TbRI then interacts with an adaptor protein SARA (Smad anchor for receptor activation) (Tsukazaki et al., Cell, 1998, 95:779-91), which propagates signals to the intracellular signaling mediators SMAD2 and SMAD3 (Derynck et al., Cell, 1998, 95:737-40). After association with SMAD4, the SMAD complexes translocate to the nuclei, where they activate specific target genes through cooperative interactions with DNA and other DNA-binding proteins to control tumor progression (Chen et al., Nature, 1996, 383:691-6; Zhang et al., Nature, 1996, 383:168-72).

### ERβ Function as a Biomarker for RCC Progression

The present study is the first report showing that ERβ can enhance TGFβ-1/miR145, SMAD3 and the EMT pathway to control RCC invasion in vitro and in vivo. Based on IHC results in RCC samples, high ERβ expression plays a promoting role in RCC. The low level of ERβ expression generally found in low-grade tumors may predispose them to additional carcinogen exposure and in this way contribute to tumor progression. Importantly, in the later stage, ERβ levels were observed to be elevated in a significant proportion of high grade tumors compared to low-grade tumors. Thus, ERβ represents a marker of RCC progression. The present study has linked the ERβ signal with TGFβ1/miR145 and EMT signal pathways, which have high clinical significance.

Tamoxifen Treatment May not Always have a Satisfactory Efficacy in RCC Patients.

In the 1990's clinical trials, results showed there was no significant therapeutic efficacy of the anti-estrogen, tamoxifen, in advanced RCC. The present study indeed provides a solution to explain why tamoxifen treatment may not have a satisfactory efficacy with RCC patients. (i) The present study clearly showed that some RCC tissues are ERβ negative and some are ERβ positive. The anti-estrogen therapy should be only applied to ERβ (+) RCC patients. (ii) Tamoxifen is not the most effective antagonist for ERβ. Compared to ICI 182,780 and an ERβ selective antagonist (PHTPP), tamoxifen only has ˜40% efficacy to inhibit ERβ activity (FIG. 9). Together, the present study provides new insights to facilitate the redesign of using anti-estrogen therapy on ERβ(+) RCC patients.

### Example 2: Infiltrating Neutrophils Promote Renal Cell Carcinoma Progression Via VEGFa/HIF2α and Estrogen Receptor β Signals

Neutrophils make up a significant portion of the infiltrated immune cells found in the tumor microenvironment. Here it is demonstrated that there are more infiltrated neutrophils in human renal cell carcinoma (RCC) lesions than adjacent benign areas. In vitro RCC studies showed that neutrophils (HL-60N cells) infiltrated toward RCC cells and subsequently enhanced RCC cell migration and invasion. Co-culture of RCC cells with HL-60N cells up-regulated ERβ, VEGFa and HIF2α mRNA levels. ERβ signals increased RCC cell migration via induction of the VEGFa/HIF2α pathway. Treatment of HIF inhibitor or rapamycin, or knockdown of ERβ in RCC cells reversed HL-60N-promoted RCC migration. In vivo data using orthotopically xenografted RCC mouse model confirmed that infiltrated neutrophils promoted RCC migration via modulating the expressions of signal pathways. These studies reveal that neutrophils are favorably recruited to the RCC cells to promote the RCC migration and invasion. Targeting the infiltrating RCC tumor microenvironment with anti-estrogen or rapamycin is a potential therapy to suppress RCC progression.

The materials and methods employed in these experiments are now described.

Human Samples and IHC Staining

To investigate the expression of CD66G+ neutrophils in human RCC tissues, malignant tissue specimens and 15 adjacent normal tissues were collected from 60 clear cell RCC patients who had undergone partial or radical nephrectomy for primary RCC at the Department of Urology, the First Affiliated Hospital of Medicine School, Xi'an Jiaotong University, between 2002 and 2012. All tumor tissues were diagnosed according to the 2009 edition of the TNM system and nuclear grading was performed according to WHO guidelines.

Formalin-fixed, paraffin-embedded samples were cut to a thickness of 5 μm. Each tissue section was deparaffinized and rehydrated with graded ethanol. For antigen retrieval, the slides were put in 1 mM EDTA solution (pH 8.0) and boiled in a microwave oven for 15 min. Endogenous peroxidase activity was blocked with a 0.3% hydrogen peroxide solution for 10 min at room temperature. After rinsing with PBS, slides were incubated overnight at 4° C. with respective primary antibodies which include mouse anti-human CD66b monoclonal antibody (ThermoFisher; dilution 1:50). After three washes in PBS, sections were incubated with biotinylated anti-mouse secondary antibody for 30 min at room temperature. Immunostaining was performed using the Envision System with diaminobenzidine (DakoCytomation, Glostrup, Denmark). Finally, the signal was developed with 3,3′-diaminobenzidine tetrahydrochloride (DAB), and all of the slides were counterstained with hematoxylin. Data were obtained by manually counting positively stained cells in five separate areas of intratumoral regions under 400× high-power magnification. Densities were determined by computing the mean number of positively stained cells per high power microscopic field (Chen et al., Int J Biol Sci, 2011, 7:53-60; Zhao et al., PLoS One, 2012, 7:e33655).

Cell Culture and Stable Cell Lines

The human RCC cell lines 786-O (with high endogenous ERβ level), and A498 (with low endogenous ERβ) were purchased from ATCC and grown in DMEM media containing 1% antibiotics and 10% fetal bovine serum (FBS) and maintained in 10% heat-inactivated FBS RPMI media with 1% Pen/Strep. Normal human kidney proximal tubular cell lines, HKC-2/HKC-8 (ERβ positive) were kindly provided by Dr. Syed Khundmiri from University of Louisville (Louisville, Ky.), and maintained in Dulbucco's Minimum Essential Medium (DMEM) (Invitrogen, Carlsbad, Calif.) with 10% fetal bovine serum (FBS). The ERβ knockdown of 786-O and ERβ overexpressing A498 cells were established by lentiviral transduction of siRNA ERβ or ERβ cDNA, respectively.

### Lentiviral Expression Plasmid Construction and Virus Production

ERβ (PLKO.1-puro-shERβ) was constructed with target sequence 5′-GCGAGTAACAAGGGCATGGAA-3′ (SEQ ID NO: 1) according to Addgene's pLKO.1 protocol. Lentiviral particles were generated by calcium phosphate transfection of lentiviral expressing, packaging and envelop plasmids into HEK 293 cells. Lentiviral particles were collected to infect target cells according to previous reports (Hsu et al., Carcinogenesis, 2014, 35:651-661).

The 786-O and A498 cell lines were stimulated with 1.25% DMSO for 2 weeks to be differentiated into neutrophils as HL-60N. All cell lines were cultured in a 5% (v/v) CO2 humidified incubator at 37° C.

Cell Migration Assay

The RCC cells and normal human kidney proximal tubular cell lines, HKC-2 and HKC-8, at 1×10⁵ were plated into the lower chambers of the transwells with 5 μm pore polycarbonate membrane inserts. 1×10⁵ HL-60N cells were plated onto the upper chambers. After 8 hrs, the cells migrated into the lower chambers were collected and counted by the Bio-Rad TC10 automatic cell counter. Each data point was performed in triplicate and the experiments were independently repeated twice.

The migration capability of RCC cells was determined using the transwell assay. RCC cells were co-cultured with HL-60N for 2 days, then trypsinized and seeded with serum-free DMEM into the upper chambers at 2-5×104 cells/well, the bottom chambers contained DMEM with 10% FBS, and incubated for 24 hr. The migrated RCC cells attached to the lower surface of the membrane were fixed by 4% paraformaldehyde and stained with 1% toluidine blue. Cell numbers were counted in five randomly chosen microscopic fields per membrane.

Invasion Assay

For in vitro invasion assays (Slavin et al., Carcinogenesis, 2014, 35:1301-1309), the upper chambers of the transwells (8 μm pore size) were pre-coated with the growth factor-reduced matrigel (matrigel: serum free RPMI=1:4) (BD Biosciences). Prior to the invasion assays. RCC cells were co-cultured with HL-60N for 72 hrs in 6-well transwell plates (0.4 μm pore size). 1×10⁵ of HL-60N cells were plated onto the upper chambers and 1×10⁶ RCC cells were plated into the lower chambers. The conditioned media (CM) were collected, diluted with 10% FBS DMEM media at 1:1 ratio, added into the lower chambers, and the parental RCC cells (1×10⁵) with or without indicated treatment were plated onto upper chambers. After 72 hrs of incubation, the cells in the upper chamber were removed. The insert membranes were fixed in ice cold 75% alcohol, stained with crystal violet, and the positively stained cells were counted. The invaded RCC cell numbers were averaged from counting numbers of five random fields. Each data point was run in triplicate and each set of experiment was performed in triplicate.

Quantitative PCR

Total RNA was extracted from each cell line using Trizol (Invitrogen). Reverse transcription was performed using the iScript reverse transcription kit (Bio-Rad). Quantitative real-time PCR (qRT-PCR) was conducted using a Bio-Rad CFX96 system with SYBR green to determine the levels of mRNA expression of listed genes. Expression levels were normalized to the expression of GAPDH mRNA.

Western Blot Assay

Cells were washed twice in PBS and lysed with RIPA buffer containing 1% protease inhibitors (Amresco, Cochran, USA). Protein concentration in the cell lystate solution was determined by BCA protein assay (Amresco, Cochran, USA). The cell lystate was mixed with 5×SDS-PAGE loading buffer (Amresco). Equivalent protein quantities were loaded to 7%-15% SDS-polyacrylamide gels (Bio-Rad). Proteins were electotransferred to PVDF membranes (Millipore, Atlanta Ga., USA) that were blocked in Tris-buffered saline plus 0.05% Tween-20 (TBS-T) containing 5% non-fat dried milk for 1 hr. The membranes were washed in TBS-T and incubated with each primary monoclonal antibody overnight at 4° C. (Chen et al., J Pathol, 2012, 226:17-27). The following primary antibodies were used: Rabbit anti-ERβ polyclonal antibody (GTX 110607, GeneTex), rabbit anti-VEGF polyclonal antibody (Abcam ab46154, 1 μg/ml) and mouse anti-HIF2α monoclonal antibody (Abcam ab8365, 1:200 diluted) and mouse anti-GAPDH monoclonal antibody (Santa Cruz) were used at 1:1000 dilution. The immune-positive bands were visualized with an ECL chemiluminescent detection system (Thermo Scientific), and the images were transferred to the Bio-rad imaging system. All analyses were performed at least in duplicate.

Rapamycin Suppresses Neutrophil Infiltration Toward RCC Cells and Neutrophils-Increased RCC Invasion

786-O (or HK2) cells were seeded in 6-cm cell culture dishes (1×10⁵/dish). After 24 hrs, media were refreshed and mock control or rapamycin (10 ng/ml) was added. After 48 hrs, CMs were collected for the following neutrophil recruitment assays. The CM collected from HK2 (or 786-O) cells with/without rapamycin treatment were passed through 0.45 μM filters. CMs were then added in the bottom wells of 24-well transwells. Neutrophils were seeded into the upper wells of transwells with serum free media (1×10⁵ in 150 μl media/well, pore size: 5 μm). After 3 hrs of incubation, infiltrated neutrophils in the media of the bottom wells were collected and counted.

For in vitro invasion assays, the upper chambers of the transwells (Corning; pore size: 8 μm) were pre-coated with matrigel containing reduced-growth factors (1:4 serum free RPMI media) (BD Biosciences, Sparks, Md.). Before the invasion assays, RCC cells were cultured alone or co-cultured with HL-60N for 48 hrs in 6-well transwell plates (Corning; pore size: 0.4 μm) with/without rapamycin (10 ng/ml) treatment. 1×10⁵ of HL-60N cells were plated onto the upper chambers and 1×10⁶ RCC cells were seeded into the lower chambers. 5 types of rapamycin treatments were tested: (i) HL-60N cells were treated with rapamycin for 24 hrs, then cells were rinsed with new media twice to wash out the excess rapamycin, then co-cultured with 786-O; (ii) HL-60N and 786-O cells were both treated with rapamycin for 24 hrs, then rapamycin was washed out, and then cells remained co-cultured together; (iii) only 786-O cells were treated with rapamycin before co-culture; and (iv) HL-60N and 786-O cells were co-cultured together in the presence of rapamycin for 48 hrs. The conditioned media (CM) were collected, diluted with 10% FBS DMEM media at the ratio of 1:1, plated into the lower chamber, and the parental RCC cells (1×10⁵) without treatment were plated onto upper chamber of transwell plate. After 18 hrs of incubation, the un-invaded cells in the upper chamber were removed. The insert membranes were fixed in ice cold 75% alcohol, stained with crystal violet, and the positively stained cells attached to the bottom of the membranes were counted under the microscope. The numbers of invaded cells were averaged from counting of five random fields. Each sample was run in triplicate and in at least 3 independent experiments.

In Vivo Metastasis Studies

Female nude mice (7-8 weeks old) were purchased from NCI. Group 1 mice were injected with 1×10⁶ 786-O cells (mixed with Matrigel, 1:1 v/v) and Group 2 mice were co-implanted with 1×10⁶ 786-O cells and 1×10⁵ HL-60N cells into renal capsule, (n=5/each group). At the end of experiments, the tumors grown in the renal capsule were harvested, measured, and fixed for further histopathological analysis. Metastatic foci were counted and collected (Slavin et al., Carcinogenesis, 2014, 35:1301-1309).

IHC for Animal Model

The mouse RCC tumor samples were fixed in 4% neutral buffered paraformaldehyde, and embedded in paraffin. The primary antibodies of the mouse anti-human CD66b monoclonal antibody (Thermo Fisher), mouse monoclonal antibody to ERβ (Abcam 14C8 1:50), rabbit anti-VEGF polyclonal antibody (Abcam ab46154, 1:1000) and mouse anti-HIF-2a monoclonal antibody (Abcam ab8365, 1:1500 diluted) were used for staining. The primary antibody was recognized by the biotinylated secondary antibody (Vector), and visualized by VECTASTAIN ABC peroxidase system and peroxidase substrate DAB kit (Vector) (Hsu et al., Oncotarget, 2014, 5:7917-7935).

Statistical Analysis

Data are presented as mean±SD from at least 3 independent experiments. Statistical analyses involved paired t-test with SPSS 17.0 (SPSS Inc., Chicago, Ill.). For in vivo studies, measurements of tumor metastasis among the three groups were analyzed through one-way ANOVA coupled with the Newman-Keuls test. P<0.05 was considered statistically significant.

The results of the experiments are now described.

RCC has a Better Capacity than Surrounding Normal Kidney Tissues to Recruit Neutrophils

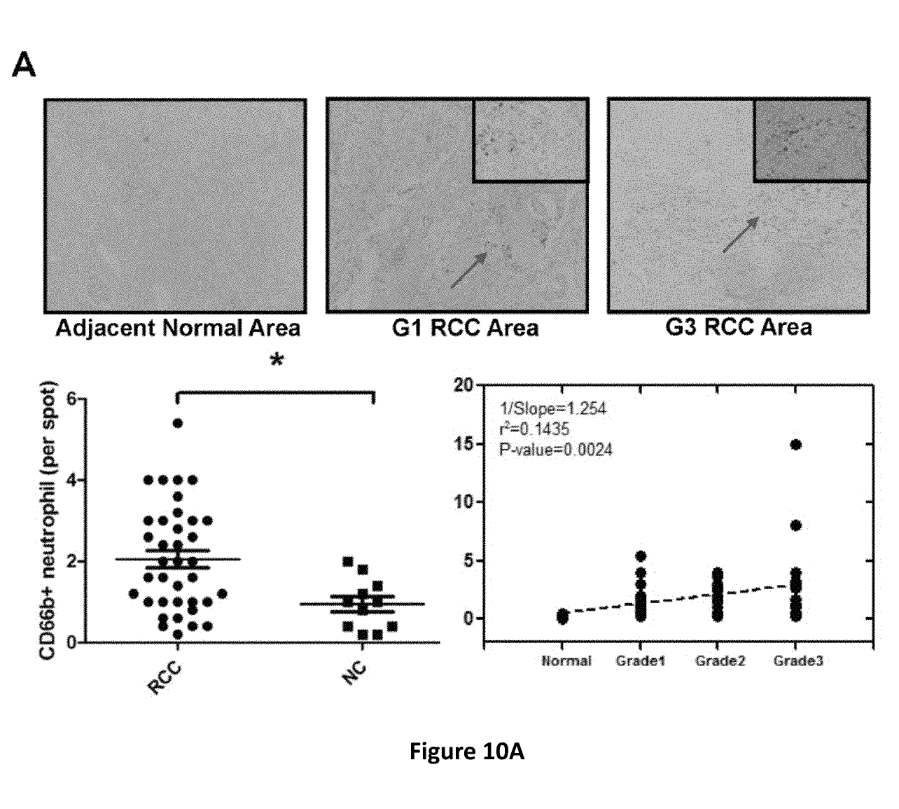

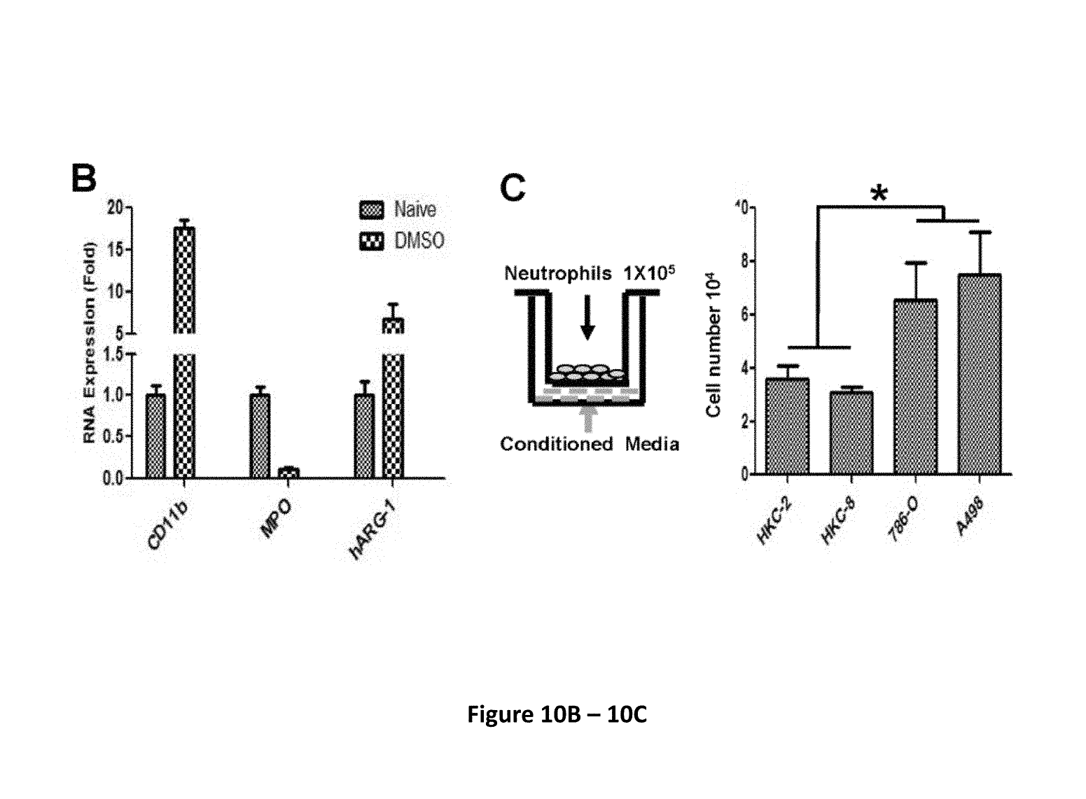

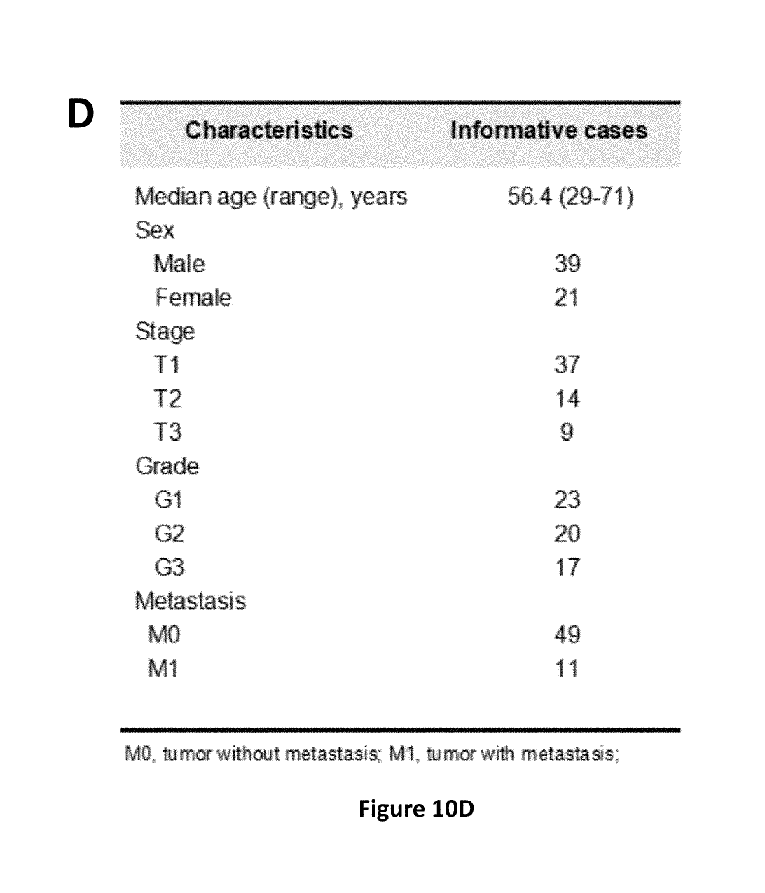

To examine the potential impacts of neutrophils on RCC progression, IHC staining was used with neutrophils marker CD66b+ to compare neutrophil infiltration and results showed that more CD66b+ neutrophils were recruited to the RCC lesions than in normal kidney tissues (FIG. 10A).

The in vitro migration assay was then used to confirm the above human clinical data. HL-60 cells were differentiated to neutrophil-like cells, HL-60N, by treating HL60 cells with 1.25% DMSO for 5 days. Neutrophil markers, CD11b and MPO, and N2 type marker, hARG-1, were detected to validate the differentiation of N2 type neutrophils (HL-60N) (FIG. 10B). To test whether RCC cells have a better capability than the non-malignant kidney cells to attract neutrophils, a transwell Boyden chamber migration system was applied. HL-60N cells were placed on the top wells, RCC or non-malignant kidney cells were seeded on the bottom wells (FIG. 10C). After 8 hours of incubation, the number of HL-60N cells that migrated through the membranes were counted. Compared to the non-malignant kidney cells, HKC-2 or HKC-8, the RCC cells, 786-O and A498, have a much better capacity to recruit the HL-60N cells (FIG. 10C).

Together, results from FIG. 10A-10C demonstrate that RCC cells/tissues have a better capacity to recruit neutrophils than its surrounding normal kidney cells.

Infiltrated Neutrophils to RCC could Enhance the RCC Cell Migration/Invasion Via Up-Regulation of ERβ Signaling in RCC Cells

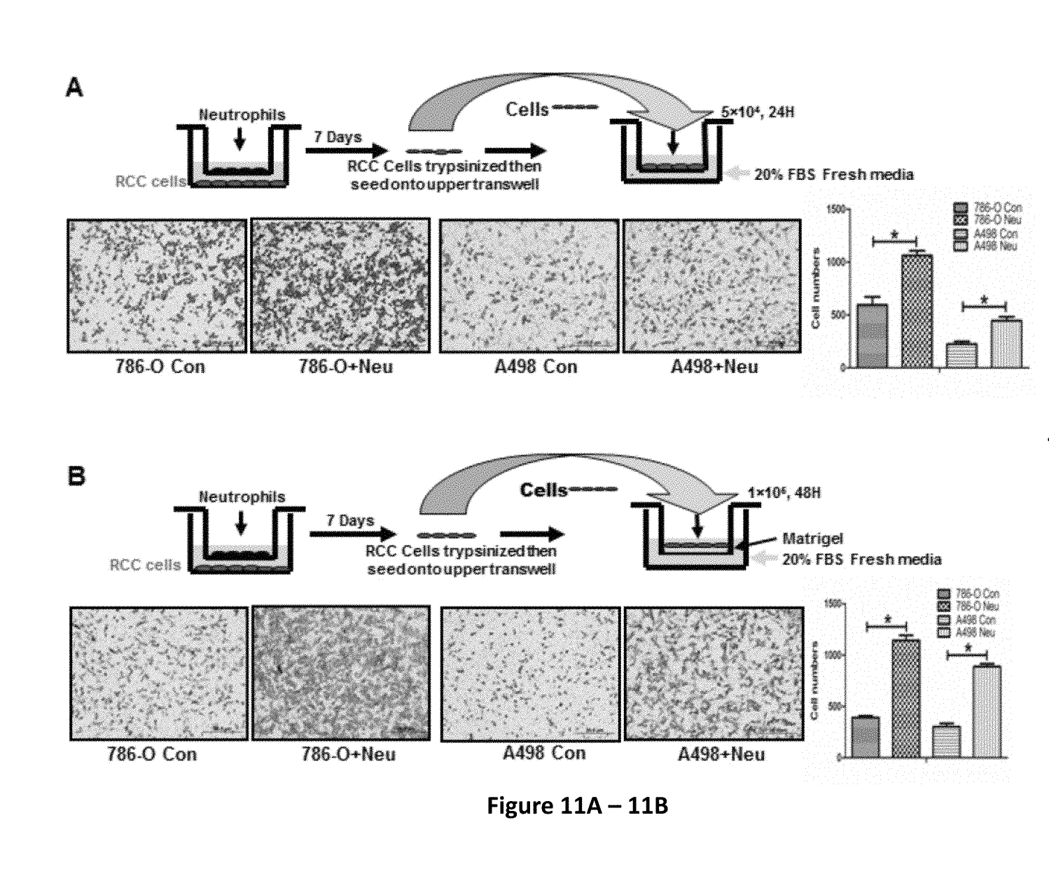

To further study the consequences of infiltrated neutrophils on RCC progression (FIG. 11A), transwell plates were used to test the migration/invasion of RCC cells with or without co-culturing with neutrophils HL-60N cells for 7 days. RCC cells were then re-seeded in the upper transwell (5×10⁴/well). The migration results showed the higher ability of migration in neutrophil-co-cultured RCC cells more than non-co-cultured RCC cells (FIG. 11A). In addition, the transwell matrigel assay results showed that co-culture of infiltrated HL-60N cells would allow RCC 786-O cells to gain a better invasion capacity (FIG. 11B, *P<0.05). Similar results were obtained when RCC 786-O cells were replaced with the A498 cells, another RCC cell line.

### Mechanism(s) how Infiltrated Neutrophils Modulate ERβ Expression in RCC Cells

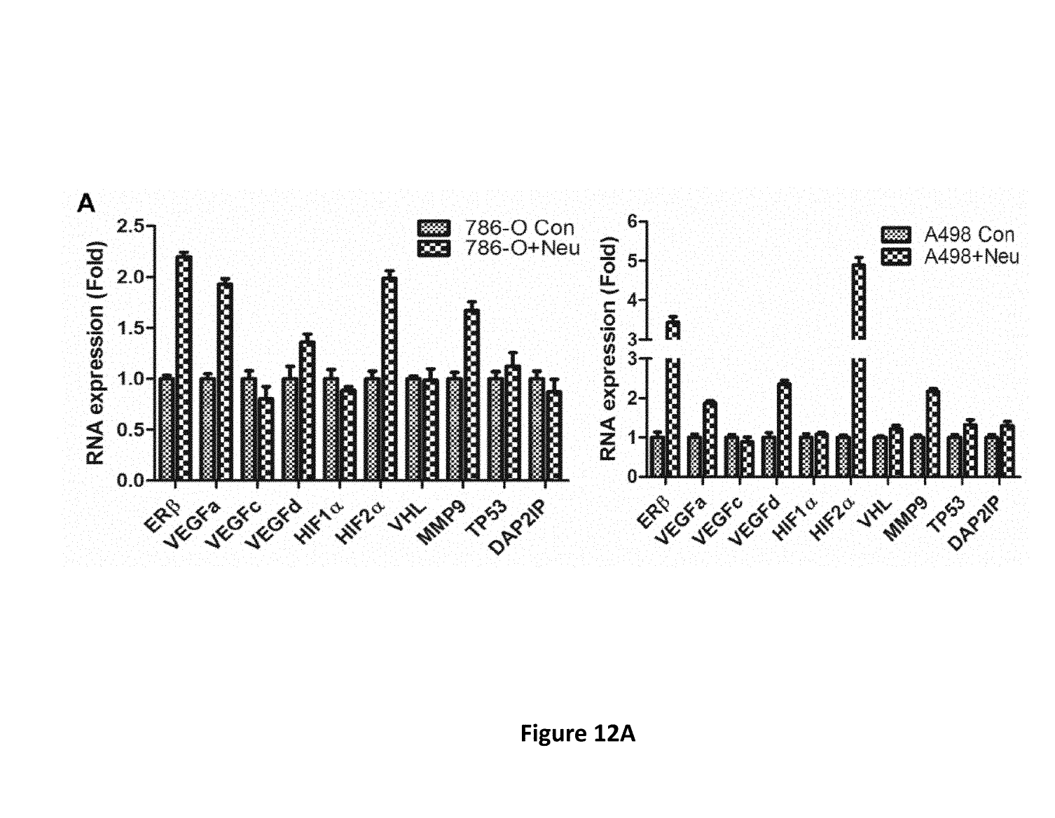

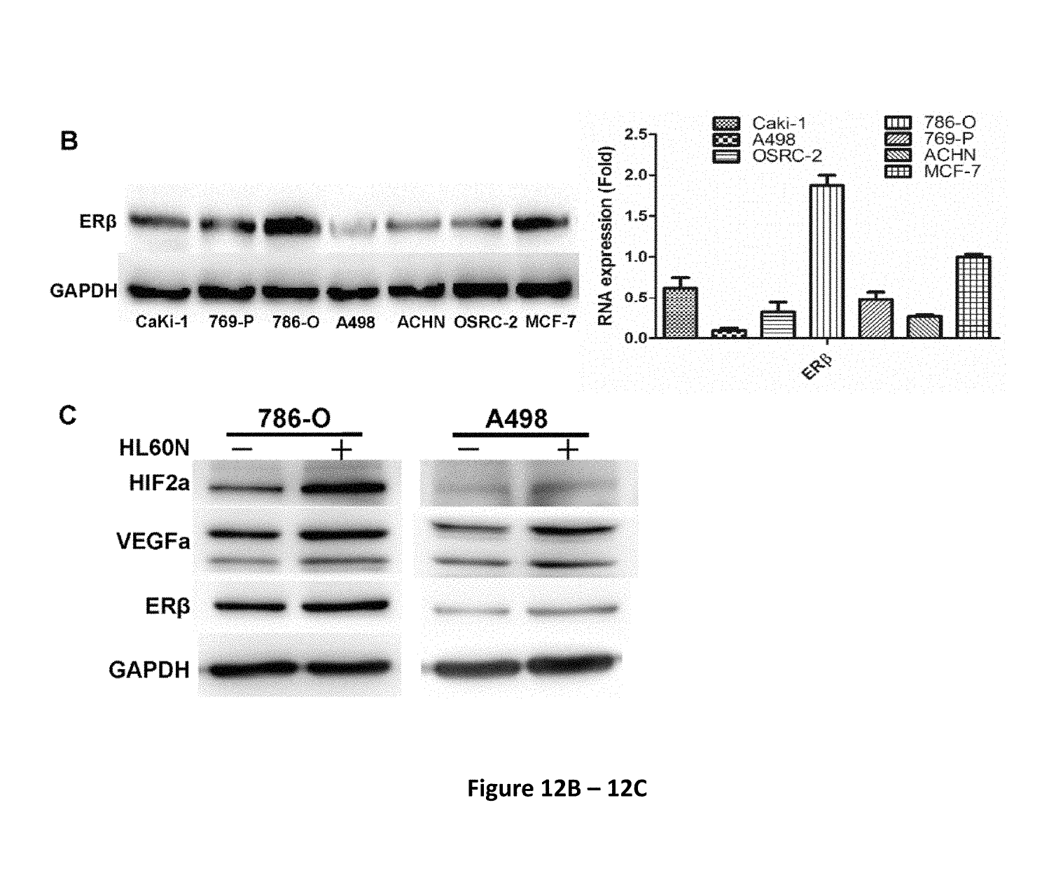

To further dissect the molecular mechanism(s) by which RCC cell invasion is enhanced after co-culture with neutrophil HL-60N cells, Q-PCR-based focus-array analyses were used to search for the key metastasis-related genes that are responsible for ERβ-enhanced RCC progression. Among many increased metastasis-related genes, the expression of HIF2α and vascular endothelial growth factor a (VEGFa) were found to be increased by the ERβ expression in RCC 786-O and A498 cells (FIG. 12A). Consistent with mRNA level changes, using Western-Blot analyses showed the up-regulation of ERβ in FIG. 12B, and in FIG. 12C showed the increased HIF2α, VEGFa and ERβ protein expressions in RCC cells after co-culture with HL-60N cells.

Together, results from FIGS. 11 and 12 using different RCC cell lines demonstrated that recruited neutrophils could enhance the RCC cell migration/invasion and infiltrated neutrophils may promote RCC cells invasion via up-regulation of ERβ signals in RCC cells.

### Knockdown of ERβ, and Treatment of HIF Inhibitor or Rapamycin can Inhibit Neutrophils-Promoted RCC Invasion

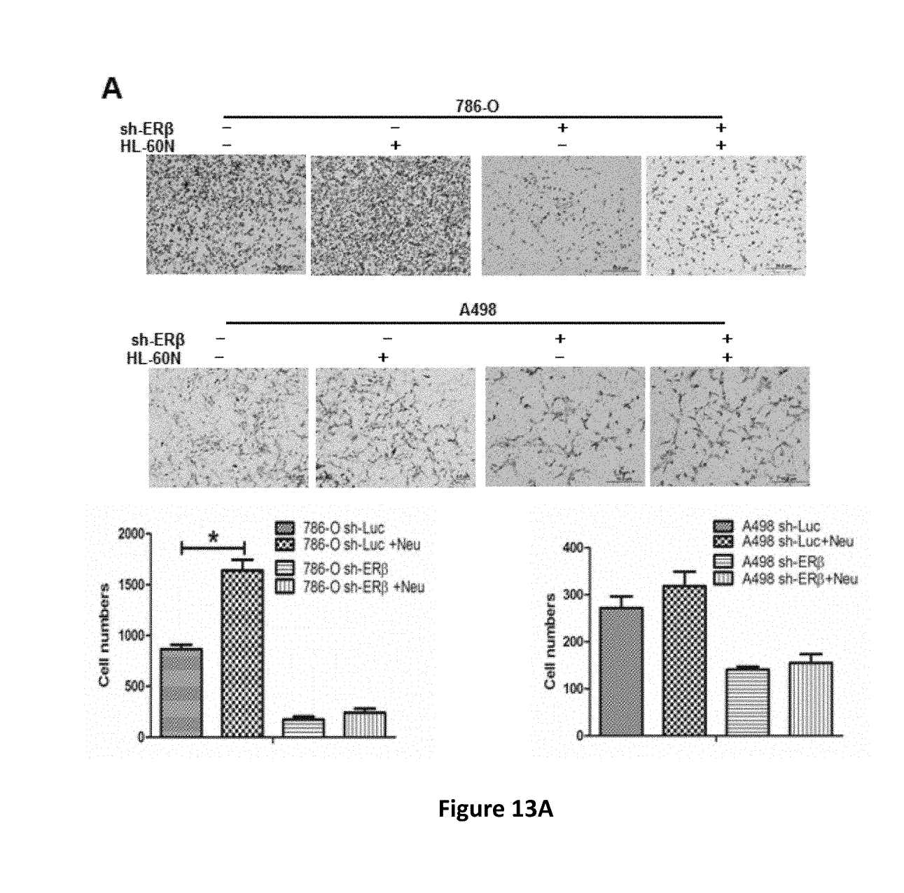

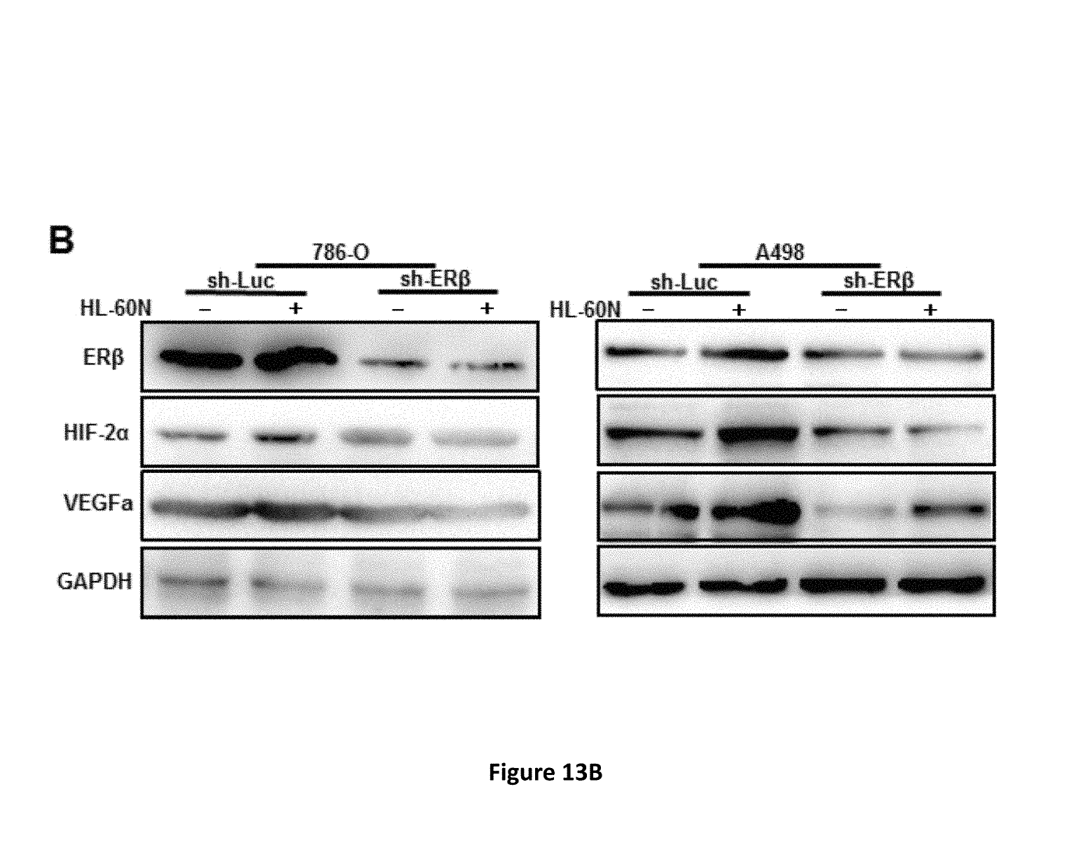

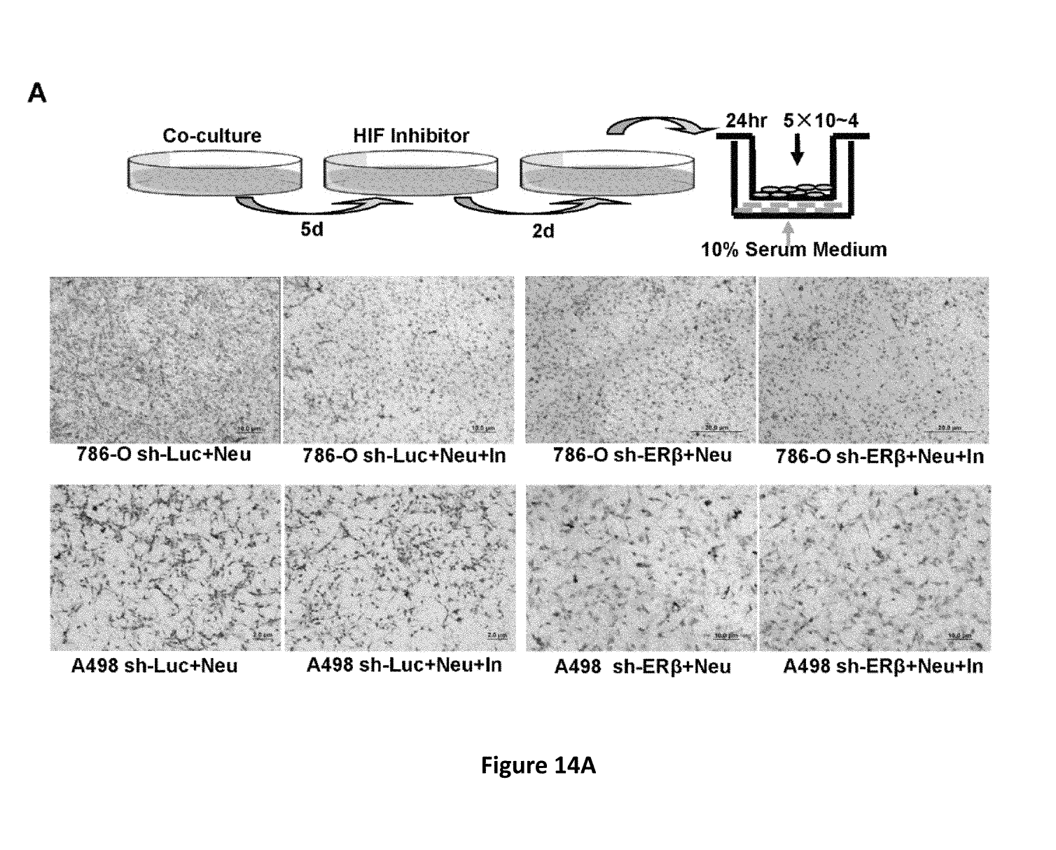

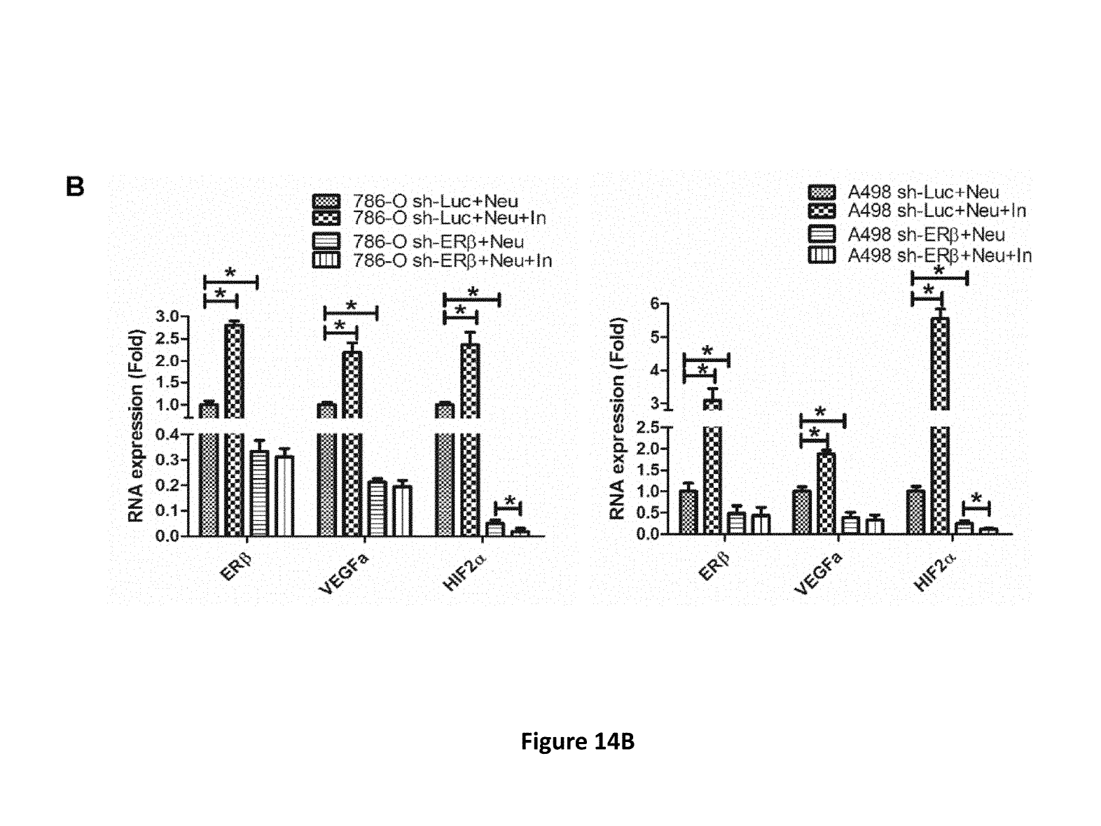

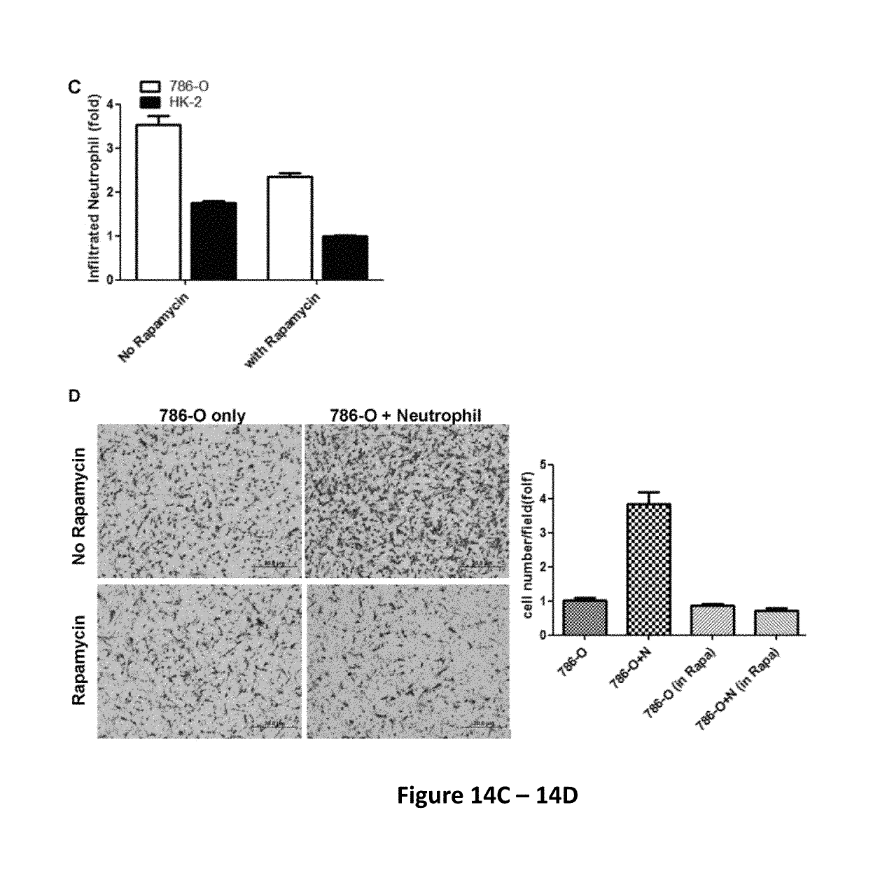

To validate the importance of ERβ, VEGFa and HIF2α in neutrophils promoted RCC invasion, lentiviral-ERβ or vector control transduced RCC cells were used. RCC cells were then co-incubated with neutrophils for 2 days and seeded for invasion assay. The data showed that knockdown of ERβ in RCC cells could inhibit neutrophils-promoted RCC invasion. Interestingly, when ERβ was knocked down, a reduced expression of the VEGFa and HIF2α in HL-60N co-cultured RCC cells was observed (FIG. 13). An interruption approach using HIF inhibitor can effectively reverse neutrophil-co-culture induced HIF2a expression and invasion in RCC cells (FIGS. 14A and 14B).

In addition to knocking down ERβ and treatment of HIF inhibitor, the effects of rapamycin that could inhibit the migration of neutrophils (Luan et al., Kidney Int, 2003, 63:917-926), or macrophages (Wu et al., Cell Res, 2012, 22:1003-1021) were examined. Rapamycin has the potential to be used as treatment of different types of cancers (Knoechel et al., Nat Genet, 2014, 46:364-370; Mulholland et al., Cancer Cell, 2011, 19:792-804). It was tested whether rapamycin could interrupt the infiltration of neutrophil toward RCC cells as well as the neutrophils-enhanced RCC invasion. Results showed that rapamycin treatment could effectively inhibit the capability for neutrophils to infiltrate through coated transwell-membrane (FIG. 14C) and consequently inhibit the neutrophils-enhanced RCC invasion (FIG. 14D).

Together, results from FIGS. 13 and 14 demonstrate that infiltrated neutrophils may function through modulation of ERβ/VEGFa/HIF2α signals to enhance the RCC cell invasion. The inhibition of neutrophils by rapamycin, or by blocking ERβ and HIF2a may be applied as alternative therapy strategies to control RCC invasion.

### Infiltrated Neutrophils Enhanced RCC Invasion in the In Vivo Mouse Model

To confirm the above in vitro results from various RCC cell studies in the in vivo pre-clinical RCC model, RCC 786-O and/or HL-60N cells (9:1 ratio) were orthotopically implanted under the renal capsule to test the tumor growth and metastasis.

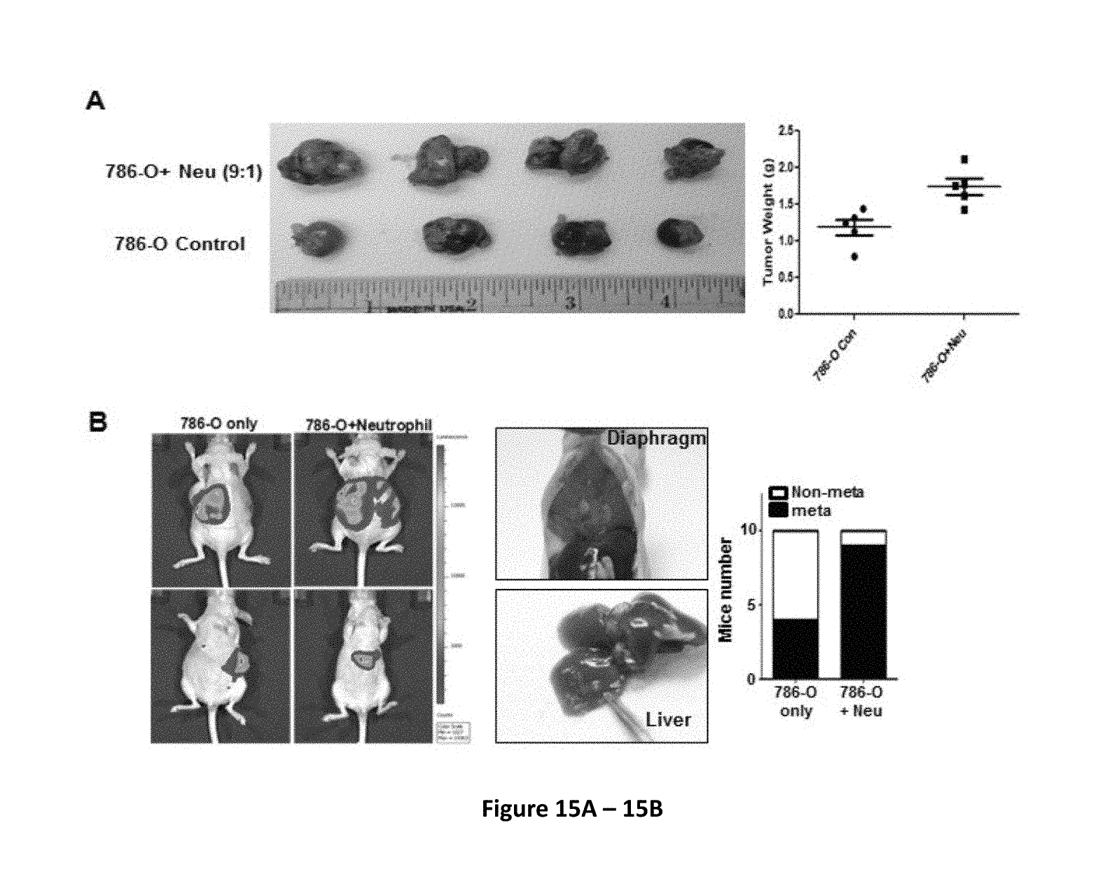

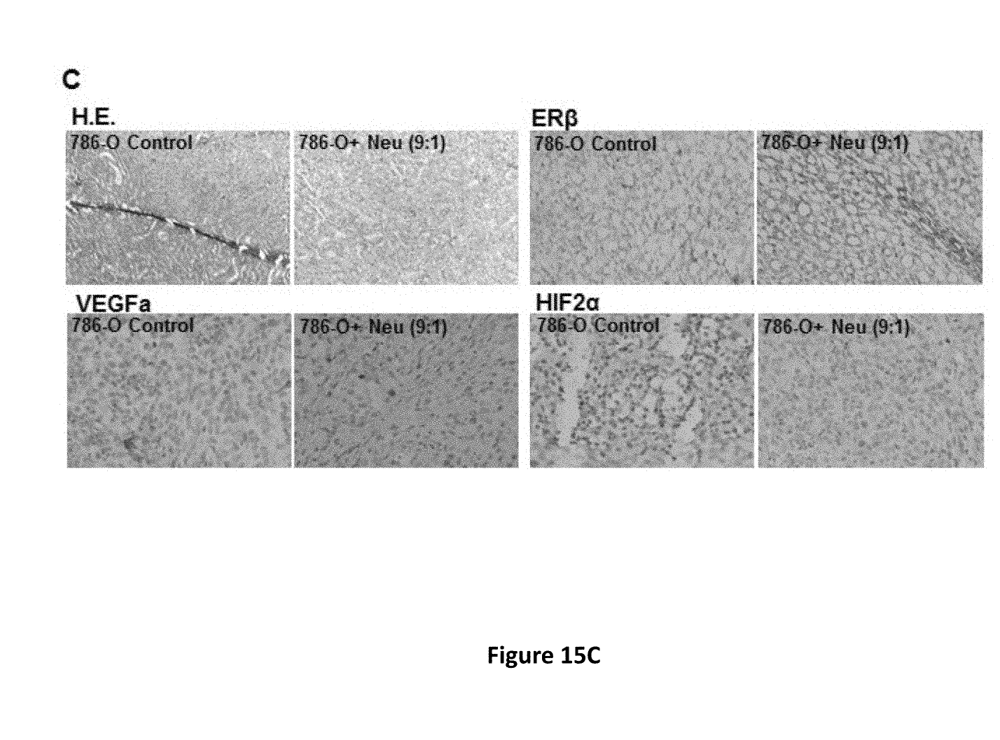

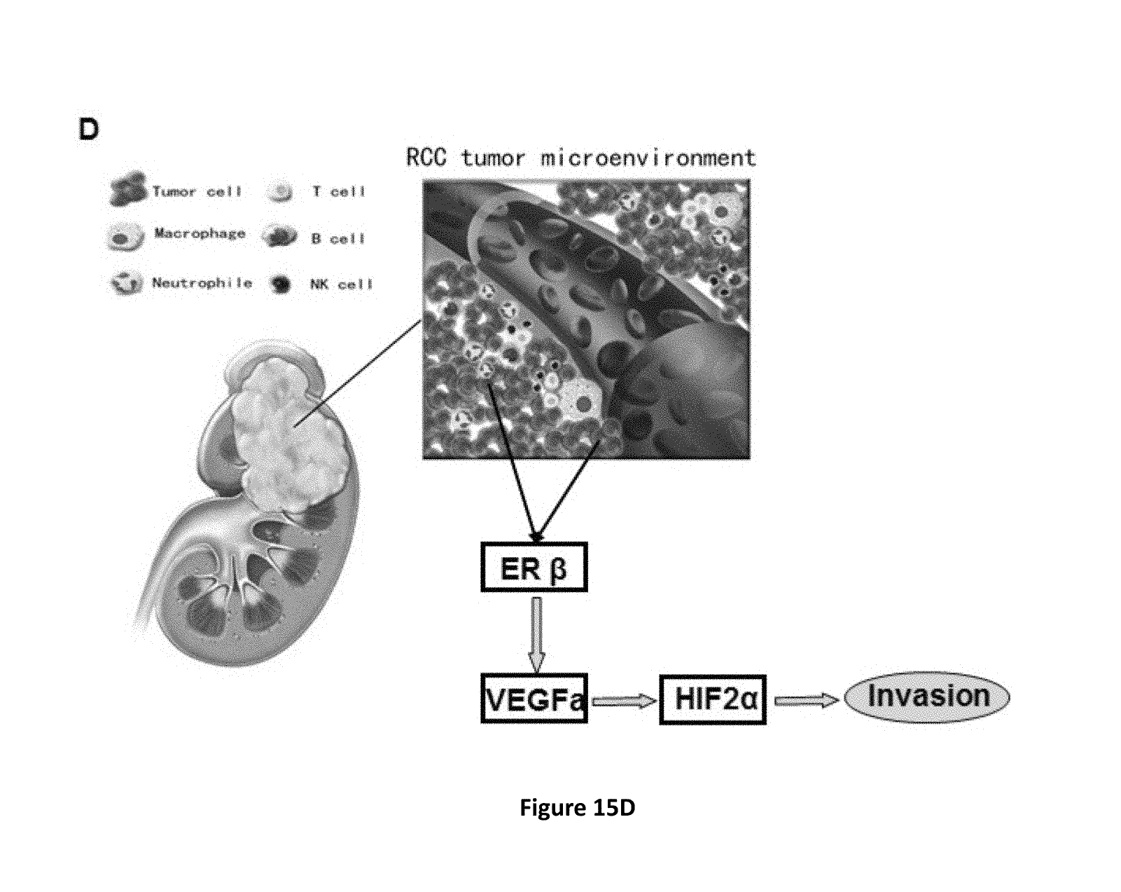

RCC 786-O cells were first stably transfected with luciferase and IVIS imaging was applied to monitor RCC tumor growth and metastasis 3 weeks after tumor implantation, followed by weekly IVIS detection for additional 5 weeks. Eight weeks after tumor implantation, the mice were sacrificed for tumor characterization. Results consistently showed that tumors were bigger in the mice co-implanted with 786-O cells and HL-60N cells (FIG. 15A). Representative IVIS images are shown on the left panel of FIG. 15B. Metastases were found in the diaphragm of neutrophils co-implanted RCC tumors. There are higher metastatic rates in 786-O+HL-60N co-implanted mouse tumor group (9/10) as compared to 786-O cell only group (4/6) (FIG. 15B). In addition, IHC staining of mouse RCC tumors showed the expressions of ERβ, VEGFa, and HIF2α markers were consistently higher in 786-O+HL-60N cells group than 786-O cells only group (FIG. 15C).

Together, results from in vivo mouse model studies (FIG. 15A-15C) confirmed the above in vitro cell lines studies (FIGS. 12 and 13) and demonstrated that infiltrated neutrophils could enhance RCC growth and invasion via modulating ERβ and VEGFa/HIF2α signals.

### Infiltrating Neutrophils Promote Renal Cell Carcinoma Progression Via VEGFa/HIF2α and Estrogen Receptor β Signals

Besides tumor cells, the tumor microenvironment is composed of a wide spectrum of immune cell types, which can significantly affect cancer progression and patient outcome. Tumor-infiltrating neutrophils (TIN) are known to make up a significant part of the immune cells within the tumor microenvironment in different types of human cancer (Iida et al., Oncol Rep, 2011, 25:1271-1277; Khayyata et al., Modern Pathol, 2005, 18:1504-1511; Queen et al., Cancer Res, 2005, 65:8896-8904). Despite their origin in the peripheral blood, TINs have been shown to exhibit impaired bactericidal and enhanced angiogenic activities (Ardi et al., P Natl Acad Sci USA, 2007, 104:20262-20267). The presence of intra-tumoral neutrophils has been reported to be associated with poor prognosis in primary breast cancer (Yamashita et al., J Leukocyte Biol, 1995, 57:375-378) and RCC (Jensen et al., J Clin Oncol, 2009, 27:4709-4717). Consistently, within these studies, neutrophil depletion experiments led to an inhibited tumor growth (Pekarek et al., J Exp Med, 1995, 181:435-440), limited metastasis numbers (Tazawa et al., Am J Pathol, 2003, 163:2221-2232) and reduced endothelial recruitment to the tumors (Sparmann et al., Cancer Cell, 2004, 6:447-458). The results confirm the rather anti-tumor role of neutrophils as significantly higher ratios of these cells were found in dissociated tumor cell suspension from advanced RCC patients with T3-T4 and metastatic disease. The study shows that RCC patients have higher proportions of neutrophils in tumor samples than in adjacent normal tissues. Higher percentages of neutrophil cells were found in the tumor tissues in the high grade RCC patients (FIG. 10). These findings may reflect the possibility of cancer-induced systemic as well as local immunosuppression, which both seem to be the early event in the course of the disease.

This study sought to determine the role of neutrophils on RCC progression and whether ERβ plays differential functions between immune cell and RCC cells. The crosstalk between immune cells and tumor cells that leads to phenotypic alterations in tumor biology has been broadly termed immunosculpting or immunoediting (Reiman et al., Semin Cancer Biol, 2007, 17(4):275-287). Previously, it was found that ERβ can promote RCC progression via TGF-β/SMAD3 pathway (Song and Yeh, et al, 2015 paper submission). A very recent study showed that ligand-bound ERβ promote prostate cancer epithelial-mesenchymal transition via VEGF pathway (Leung et al., Endocr-Relat Cancer, 2010, 17:675-689).

The VEGF pathway is an important regulator of angiogenesis (Carmeliet, Oncology, 2005, 69:4-10). Activation of this pathway triggers a network of signaling processes that promote endothelial cell growth, migration, and survival from preexisting vascular beds. The VEGF/VEGF-receptor axis plays an important role in the mobilization of endothelial progenitor cells from the bone marrow to distant sites of neovascularization. The well-established role of VEGF in promoting tumor angiogenesis and growth has led to the development of various biologic agents that target this pathway (Carmeliet, Nature, 2005, 438:932-936; Choueiri et al., Semin Oncol, 2006, 33(5):596-606). RCC may be used as a model for the development of effective antiangiogenic therapies. RCC pathogenesis may be associated with several pathways, including the inactivation of the von Hippel-Lindau tumor suppressor gene (Choueiri et al., Semin Oncol, 2006, 33(5):596-606). Neutrophils play an important role in normal physiological angiogenesis. During the menstrual cycle, angiogenesis occurs in order to support the proliferation and growth of the endometrial tissue. The source of the potent pro-angiogenic factor VEGFa in these tissues was found to be from neutrophils. In fact, neutrophils expressing VEGFa could be found in the microvessels of the endometrium during the proliferative stage of the cycle when the endometrium can quadruple in thickness (Mueller et al., Fertil Steril, 2000, 74:107-112), which demonstrate the potential impacts of neutrophil secreted VEGFa in tumor progression. In the present study, the data showed that ERβ increased VEGFa activity and promoted angiogenesis-related gene expressions.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method for treating cancer associated with abnormal estrogen receptor β (ERβ) expression in a subject, the method comprising administering to the subject a therapeutically effective amount of a composition comprising an inhibitor of an estrogen receptor.

2. The method of claim 1, wherein said subject is a male.

3. The method of claim 1, wherein said cancer is renal cell carcinoma (RCC).

4. The method of claim 1, wherein said cancer is sarcomatoid type renal cancer.

5. The method of claim 3, wherein said renal cell carcinoma (RCC) is selected from the group consisting of stage II RCC, stage III RCC, and stage IV RCC.

6. The method of claim 1, wherein said cancer involves one or more cell types selected from the group of clear cells, papillary cells (Types 1 and 2), chromophobe cells, oncocytic cells, collecting duct cells, and transitional cells.

7. The method of claim 1, wherein said inhibitor is selected from the group consisting of: a nucleic acid, a siRNA, an antisense nucleic acid, a ribozyme, a peptide, a small molecule, an antagonist, an aptamer, and a peptidomimetic.

8. The method of claim 1, wherein said inhibitor is an inverse agonist to ERβ.

9. The method of claim 1, wherein said inhibitor is an antagonist to ERβ.

10. The method of claim 1, wherein said inhibitor is selected from the group consisting of: selective estrogen receptor modulator (SERM), steroidal aromatase inhibitor, and non-steroidal aromatase inhibitor.

11. The method of claim 1, wherein said inhibitor further comprises a pharmaceutically acceptable formulation that includes a carrier and optionally one or more excipients, stabilizers, or additives.

12. The method of claim 1, wherein said inhibitor is administered in combination with another therapeutic agent.

13. The method of claim 12, wherein said inhibitor is administered in combination with a TGFβ-1 inhibitor.

14. The method of claim 12, wherein said inhibitor is administered in combination with a SMAD inhibitor.

15. The method of claim 12, wherein said inhibitor is administered in combination with a HIF inhibitor.

16. The method of claim 12, wherein said inhibitor is administered in combination with rapamycin.

17. The method of claim 1, wherein said inhibitor is administered orally, topically, transdermally, parenterally, subcutaneously, intravenously, intramuscularly, intraperitoneally, by intranasal instillation, by intracavitary or intravesical instillation, intraocularly, intraarterially, intralesionally, or by application to mucous membranes.

18. The method of claim 7, wherein said inhibitor is a small molecule selected from the group consisting of 7α-[9-(4,4,5,5,5-pentafluoropentylsulfinyl)nonyl]estra-1,3,5(10)-triene-3,17β-diol (also known as ICI 182,780), N-n-butyl-N-methyl-11-(3,17β-dihydroxyestra-1,3,5(10)-triene-7α-yl)undecanamide (also known as ICI 164,384), 11β-[4-[2-(dimethylaminoethoxy]phenyl]-estradiol (also known as RU 39411), N-methyl-N-isopropyl-(3,17β-dihydroxy-estra-1,2,5(10)-trien-11-β-yl)-undecamide (RU 51625), its 17α-ethynyl derivative (RU 53637), [6-hydroxy-2(4-hydroxyphenyl)benzo[b]thien-3-yl][4-[3-(1-pyrrolidin-yl)ethoxy]phenyl]methanone (LY117018), tamoxifen, toremifene, fulvestrant, raloxifene, raloxifene hydrochloride, clomiphene, keoxifene, 3-[4-(1,2-Diphenylbut-1-enyl)phenyl]acrylic acid and SR-16388.

19. A method of determining the stage of renal cancer in a subject, the method comprising obtaining a sample comprising renal cancer cells from the subject and determining the level of expression of the ERβ in the renal cancer cells wherein elevated levels of ERβ is an indication of renal cancer progression.

20. A method of diagnosing renal cancer in a subject, the method comprising obtaining a sample comprising renal cancer cells from the subject and determining the level of expression of the ERβ in the renal cancer cells wherein elevated levels of ERβ is an indication of renal cancer.

21. A kit for preventing or delaying the onset of cancer in a subject, said kit containing a composition comprising at least one inhibitor of an estrogen receptor.

22. The kit of claim 21, further comprising one or more of a TGFβ-1 inhibitor, a SMAD inhibitor, and a HIF inhibitor.
